(19) United States
(12) Patent Application Publication
(10) Pub. No.: US 2019/0225674 A1
(43) Pub. Date: Jul. 25, 2019

(54) TOOLS FOR NEXT GENERATION KOMAGATAELLA (PICHIA) ENGINEERING

(71) Applicant: Massachusetts Institute of Technology, Cambridge, MA (US)

(72) Inventor: Timothy Kuan-Ta Lu, Cambridge, MA (US)

(73) Assignee: Massachusetts Institute of Technology, Cambridge, MA (US)

(21) Appl. No.: 16/316,980

(22) PCT Filed: Jul. 11, 2017
(86) PCT No.: PCT/US2017/041509

§ 371 Date: Jan. 10, 2019

Related U.S. Application Data
(60) Provisional application No. 62/360,731, filed on Jul. 11, 2016.

(51) Int. Cl. C07K 16/10; C12N 15/81

(52) U.S. Cl. CPC C07K 16/10; C07K 2317/14; C12N 15/815

(57) ABSTRACT

Described herein are methods and compositions for the rapid production of therapeutic molecules using an inducible cell culture system.

### RELATED APPLICATIONS

This application is a national stage filing under 35 U.S.C. § 371 of International Patent Application Serial No. PCT/US2017/041509, filed Jul. 11, 2017, which claims the benefit under 35 U.S.C. § 119(e) of U.S. provisional application No. 62/360,731, filed Jul. 11, 2016, each of which is incorporated by reference herein in its entirety.

### FEDERALLY SPONSORED RESEARCH

This invention was made with Government support under Grant No. N66001-13-C-4025 awarded by the Space and Naval Warfare Systems Center (SPAWAR). The government has certain rights in the invention.

### FIELD OF INVENTION

The invention relates to inducible cell culture systems for the rapid production of therapeutic molecules and genetic tools for generating such systems.

### BACKGROUND

One of the many challenges faced by the drug manufacturing industry is the issue of global logistics: drugs may be produced in one location and need to be distributed to multiple, sometimes remote locations, under optimal storage conditions for the drug. These factors greatly impact the cost of the drug and timing of delivering drugs to patients in need. Aside from the cost of producing the drug, which is a substantial barrier for treating patients with biologic therapies in many parts of the world, the logistics of transporting the drug to the patient can significantly increase the final cost of the product. Alternative approaches to providing drugs to individuals in remote or under-resourced regions, particularly in emergency situations where existing infrastructure has been compromised or in the battlefield, are desired.

### SUMMARY

Described herein are methods, compositions, and kits for biomanufacturing (e.g. manufacturing of therapeutic biologics) that have applications for real-time production of therapeutic molecules. The methods described herein provide a modifiable and portable platform for producing polypeptides at the point-of-care, in short timeframes (e.g. <48 hours), and can be used when a specific need arises. The platform includes at least a cell-based expression system genetically engineered to secrete one or more polypeptides (e.g., therapeutic molecules). The methods provided herein allow for production of polypeptides and eliminate the intermediate logistics steps, directly linking drug production to patients in need.

Aspects of the present disclosure provide methods for producing a polypeptide, comprising (i) providing a genetically modified cell that encodes a first inducible system at a first genetic locus of the cell, wherein the first inducible system comprises a first transcription factor, at least one binding site for the first transcription factor operably linked to a first inducible promoter, and a first recombination site downstream of the first inducible promoter; (ii) providing to the cell a plasmid that comprises a nucleotide sequence encoding a first polypeptide, optionally a first signal peptide, and a second recombination site; (iii) expressing a first recombinase compatible with the first and second recombination sites such that recombination occurs between the first recombination site of the cell and the second recombination site of the plasmid resulting in integration of the nucleotide sequence encoding the first polypeptide and optionally the first signal peptide downstream of the first inducible promoter; (iv) culturing the cell of (iii); and (v) providing an inducer for the first inducible system thereby inducing expression of the first polypeptide.

In some embodiments, the genetically modified cell encodes a second inducible system at the first genetic locus of the cell. In some embodiments, the genetically modified cell encodes a second inducible system at a second genetic locus of the cell. In some embodiments, the second inducible system comprises a second transcription factor, at least one binding site for the second transcription factor operably linked to a second inducible promoter, and a third recombination site downstream of the second inducible promoter.

In some embodiments, the method further comprises (a) providing to the cell a plasmid that comprises a nucleotide sequence encoding a second polypeptide, optionally a second signal peptide, and a fourth recombination site; (b) expressing a second recombinase compatible with the third and fourth recombination sites such that recombination occurs between the third recombination site of the cell and the fourth recombination site of the plasmid resulting in integration of the nucleotide sequence encoding the second polypeptide and optionally the second signal peptide downstream of the second inducible promoter; (c) culturing the cell of (b); and (d) providing an inducer for the second inducible system thereby inducing expression of the second polypeptide.

In some embodiments, the genetically modified cell further encodes a fifth recombination site and the plasmid further comprises a sixth recombination site. In some embodiments, the method further comprises expressing a third recombinase compatible with the fifth and sixth recombination sites such that recombination occurs between the fifth and sixth recombination sites resulting in removal of nucleic acid. In some embodiments, the first and second inducible promoters are different.

In some embodiments, the method further comprises collecting the first and/or second polypeptide. In some embodiments, the method further comprises purifying the first and/or second polypeptide. In some embodiments, purifying the first polypeptide and/or second polypeptide comprises obtaining a culture, culture supernatant or composition comprising the first polypeptide and/or second polypeptide, subjecting the culture, culture supernant or composition comprising the first polypeptide and/or second polypeptide to one or more chromatography steps to purify the first polypeptide and/or the second polypeptide. In some embodiments, the one or more chromatography steps comprise one or more of Sepharose chromatography, reverse phase chromatography, Protein A chromatography, and affinity chromatography.

In some embodiments, the cell is a yeast cell. In some embodiments, the yeast cell is a Komagataella phaffi (Pichia pastoris). In some embodiments, the first and/or the second inducible system is on chromosome 2 of the cell. In some embodiments, the first and/or the second inducible system is at the TRP2 locus of chromosome 2.

In some embodiments, the first recombinase, second recombinase, and/or third recombinase is BxbI, R4, TP-901, Cre, Flp, PiggyBac, PhiC31, Gin, Tn3, ParA, HP1, or HK022. In some embodiments, the first recombination site is an attB site, and the second recombination site is an attP site; or the first recombination site is an attP site, and the second recombination site is an attB site.

In some embodiments, the DNA binding domain of the first and/or second transcription factor is a zinc finger DNA binding domain. In some embodiments, the zinc finger DNA binding domain is ZF43-8. In some embodiments, the inducer binding domain of the first and/or second transcription factor is a β-estradiol binding domain. In some embodiments, the β-estradiol binding domain is from the human estrogen receptor. In some embodiments, the transcription activation domain of the first and/or second transcription factor is VP64.

In some embodiments, the inducer of the first and/or second inducible system is β-estradiol. In some embodiments, the β-estradiol is provided at a concentration of about 0.01 μM-1.0 μM. In some embodiments, the β-estradiol is provided for less than 48 hours. In some embodiments, 0.01 μM β-estradiol is provided for less than 24 hours.

In some embodiments, the plasmid comprises more than one nucleotide sequence encoding more than one polypeptide separated by a nucleotide sequence encoding a 2A peptide.

In some embodiments, between 1 pg and 10 g of the first and/or second polypeptide is produced. In some embodiments, the first and/or second polypeptide is a therapeutic molecule. In some embodiments, the therapeutic molecule is an antibody, hormone, cytokine, chemokine, growth factor, vaccine, or enzyme. In some embodiments, the cytokine is IFNα2b. In some embodiments, at least 19 μg of IFNα2b is produced in approximately 20 hours. In some embodiments, the growth factor is human growth hormone (hGH). In some embodiments, at least 40 μg human growth hormone is produced in approximately 20 hours.

In some embodiments, the first and or second inducible system comprises between 2-9 transcription factor binding sites located upstream of the inducible promoter in the plus orientation or the minus orientation. In some embodiments, expression of the first and/or second transcription factor is regulated by a constitutive promoter. In some embodiments, the constitutive promoter is a GAP promoter, a TEF1 promoter, a P GCW14 promoter, a variant of the GAP promoter, or a variant of the TEF1 promoter. In some embodiments, the first and/or second inducible promoter is an AOX1 promoter, a GAP promoter, a TEF1 promoter, a P GCW14 promoter, a variant of the GAP promoter, or a variant of the TEF1 promoter. In some embodiments, the variant of the TEF1 promoter is a scTEF1 promoter.

In some embodiments, the constitutive promoter is the GAP promoter and the inducible promoter is the AOX1 promoter; or the constitutive promoter is a variant of the GAP promoter and inducible promoter is the AOX1 promoter; or the constitutive promoter is the scTEF1 promoter and the inducible promoter is the GAP promoter; or the constitutive promoter is the scTEF1 promoter and the inducible promoter is a variant of the GAP promoter.

In some embodiments, the signal peptide is a yeast signal peptide. In some embodiments, the yeast signal peptide is a S. cerevisiae signal peptide. In some embodiments, the yeast signal peptide is the S. cerevisiae mating factor alpha-1 signal peptide.

In some embodiments, the first recombinase, second recombinase, and/or third recombinase are encoded on a second plasmid provided to the cell or in the genome of the cell.

In some embodiments, the culturing is performed in the presence of at least one antifoam agent. In some embodiments, the antifoam agent is L81, P2000, or antifoam 204.

Other aspects provide cells comprising a first nucleic acid encoding a first transcription factor regulated by a first constitutive promoter, at least one transcription factor binding site, a first inducible promoter, and a nucleotide sequence encoding a first polypeptide, and optionally a first signal peptide, downstream of and operably linked to the first inducible promoter, and wherein the nucleotide sequence encoding the first polypeptide and optionally the first signal peptide are flanked by a first pair of recombined recombination sites, and wherein the first nucleic acid is located at a first genetic locus, wherein the first genetic locus is in chromosome 2 of the cell.

In some embodiments, the cell further comprises a second nucleic acid encoding a second transcription factor regulated by a second constitutive promoter, at least one transcription factor binding site, a second inducible promoter, and a nucleotide sequence encoding a second polypeptide, and optionally a second signal peptide, downstream of and operably linked to the second inducible promoter, wherein the nucleotide sequence encoding the second polypeptide and optionally the second signal peptide are flanked by a second pair of recombined recombination sites, and wherein the second nucleic acid is located at the first locus of the cell. In some embodiments, the cell further comprises a second nucleic acid encoding a second transcription factor regulated by a second constitutive promoter, at least one transcription factor binding site, a second inducible promoter, a nucleotide sequence encoding a second polypeptide, and optionally a second signal peptide, downstream of and operably linked to the second inducible promoter, wherein the nucleotide sequence encoding the second polypeptide and optionally the second signal peptide are flanked by a second pair of recombined recombination sites, and wherein the second nucleic acid is located at a second locus of the cell. In some embodiments, the first and second inducible promoters are different.

In some embodiments, the cell is a yeast cell. In some embodiments, the yeast cell is a Komagataella phaffi (Pichia pastoris). In some embodiments, the first and/or the second nucleic acid is at the TRP2 locus of chromosome 2.

In some embodiments, the first and/or second polypeptide is a therapeutic molecule. In some embodiments, the therapeutic molecule is an antibody, hormone, cytokine, chemokine, growth factor, vaccine, or enzyme. In some embodiments, the cytokine is IFNα2b. In some embodiments, the growth factor is human growth hormone (hGH).

In some embodiments, the first and/or second nucleic acid comprises between 2-9 transcription factor binding sites located upstream of the first and/or second inducible promoter in the plus orientation or the minus orientation. In some embodiments, the first and/or second constitutive promoter is a GAP promoter, a TEF1 promoter, a P GCW14 promoter, a variant of the GAP promoter, or a variant of the TEF1 promoter. In some embodiments, the first or second inducible promoter is an AOX1 promoter, a GAP promoter, a TEF1 promoter, a P GCW14 promoter, a variant of the GAP promoter, or a variant of the TEF1 promoter. In some embodiments, the variant of the TEF1 promoter is a scTEF1 promoter.

In some embodiments, the first and/or second constitutive promoter is the GAP promoter and the first or second inducible promoter is the AOX1 promoter; or the first and/or second constitutive promoter is a variant of the GAP promoter and the first or second inducible promoter is the AOX1 promoter; or the first and/or second constitutive promoter is the scTEF1 promoter and the first or second inducible promoter is the GAP promoter; or the first and/or second constitutive promoter is the scTEF1 promoter and the first or second inducible promoter is a variant of the GAP promoter.

In some embodiments, the first and/or second signal peptide is a yeast signal peptide. In some embodiments, the yeast signal peptide is a S. cerevisiae signal peptide. In some embodiments, the yeast signal peptide is the S. cerevisiae mating factor alpha-1 signal peptide.

Other aspects provide methods of producing a polypeptide comprising culturing any of the cells described herein. In some embodiments, the further comprises providing a first inducer for the first inducible promoter, thereby inducing expression of the first polypeptide. In some embodiments, the method further comprises providing a second inducer for the second inducible promoter, thereby inducing expression of the second polypeptide.

In some embodiments, the inducer of the first and/or second inducible promoter is β-estradiol. In some embodiments, the β-estradiol is provided at a concentration of about 0.01 μM-1.0 μM. In some embodiments, the β-estradiol is provided for less than 48 hours. In some embodiments, 0.01 μM β-estradiol is provided for approximately 24 hours. In some embodiments, between 1 pg and 10 g of the first and/or second polypeptide is produced.

In some embodiments, at least 19 μg of IFNα2b is produced in approximately 20 hours. In some embodiments, at least 40 μg human growth hormone is produced in approximately 20 hours.

In some embodiments, the culturing is performed in the presence of at least one antifoam agent. In some embodiments, the antifoam agent is L81, P2000, or antifoam 204.

In some embodiments, the method further comprises collecting the cell culture supernatant. In some embodiments, the method further comprises purifying the first polypeptide and/or the second polypeptide from the cell culture supernatant. In some embodiments, purifying the first polypeptide and/or the second polypeptide comprises subjecting the cell culture supernatant comprising the first polypeptide and/or the second polypeptide to one or more chromatography steps to purify the first polypeptide and/or the second polypeptide. In some embodiments, the one or more chromatography steps comprises one or more of Sepharose chromatography, reverse phase chromatography, Protein A chromatography, and affinity chromatography.

Other aspects provide a cell culture produced by culturing any of the cells described herein. In some embodiments, the cell culture comprises at between 1 pg and 10 g of the first and/or second polypeptide.

Other aspects provide a genetically modified cell comprising a first inducible system comprising a first transcription factor, at least one transcription factor binding site, a first inducible promoter, and a first recombination site downstream of and operably linked to the first inducible promoter, at a first genetic locus, wherein the first genetic locus is on chromosome 2 of the cell and the cell is Komagataella phaffi (Pichia pastoris).

In some embodiments, the cell further comprises a second inducible system comprising a second transcription factor, at least one transcription factor binding site, a second inducible promoter, and a second recombination site downstream of and operably linked to the second inducible promoter, at the first genetic locus. In some embodiments, the cell further comprises a second inducible system comprising a second transcription factor, at least one transcription factor binding site, a second inducible promoter, and a second recombination site downstream of and operably linked to the second inducible promoter, at a second genetic locus.

In some embodiments, the first and/or second genetic locus is the TRP2 locus of chromosome 2. In some embodiments, the first and/or second inducible systems comprise between 2-9 transcription factor binding sites.

In some embodiments, the first or second inducible promoter is an AOX1 promoter, a GAP promoter, a TEF1 promoter, a P GCW14 promoter, a variant of the GAP promoter, or a variant of the TEF1 promoter. In some embodiments, the variant of the TEF1 promoter is a scTEF1 promoter.

Other aspects provide kits comprising (i) a genetically modified cell as described herein, (ii) a first recombinase, and (iii) a first plasmid encoding a first polypeptide, optionally a first signal peptide, and a second recombination site. In some embodiments, the kit further comprises (iv) a second recombinase, and (v) a second plasmid encoding a second polypeptide, optionally a second signal peptide and a third recombination site.

Other aspects provide methods for producing a therapeutic antibody comprising isolating B cells from infected individuals, determining the sequence of antibody variable regions from the B cells isolated from the infected individuals, synthesizing one or more antibodies using the antibody variable region sequences, engineering strains of Komagataella phaffi to express the one or more antibodies, and culturing the engineered strains of Komagataella phaffi to produce the one or more antibodies. In some embodiments, the method further comprises purifying the one or more antibodies. In some embodiments, the method further comprises screening for highly productive engineered strains of Komagataella phaffi that produce the one or more antibodies.

Other aspects provide methods for treating an infection comprising administering antibodies made by any of the methods described herein to a subject in need of such treatment.

These and other aspects of the invention, as well as various embodiments thereof, will become more apparent in reference to the drawings and detailed description of the invention.

Each of the limitations of the invention can encompass various embodiments of the invention. It is, therefore, anticipated that each of the limitations of the invention involving any one element or combination of elements can be included in each aspect of the invention. This invention is not limited in its application to the details of construction and the arrangement of components set forth in the following description or illustrated in the drawings. The invention is capable of other embodiments and of being practiced or of being carried out in various ways.

### BRIEF DESCRIPTION OF THE DRAWINGS

The accompanying drawings are not intended to be drawn to scale. For purposes of clarity, not every component may be labeled in every drawing. In the drawings:

FIGS. 1A-1C show development of an artificial promoter system for high level polypeptide expression in K. phaffi. FIG. 1A presents a schematic representation of the “landing pad” system for integrating a gene encoding a polypeptide (transgene) into a site on the genome. A parental strain was generated containing landing pads based on attB sites for the recombinases BxbI, R4, and TP-901.1. A transfer vector containing the desired transgene and the corresponding attP site together with a plasmid encoding the corresponding recombinase are then introduced into the cell harboring the landing pad, resulting in integration of the transgene into the genome. FIG. 1B shows a schematic representation of a β-estradiol inducible expression system in a cell. The system uses a zinc finger (ZF) DNA-binding domain fused to the β-estradiol-binding domain of the human estrogen receptor (ER), which is coupled to a transcriptional activation domain (VP64). At steady state, shown in the top panel, the transcription factor is sequestered in the cytoplasm of the cell by binding to HSP90. In the presence of β-estradiol (βE), the bottom panel, HSP90 is displaced and the transcription factor translocates to the nucleus where it induces expression of gene(s) (e.g., GFP) regulated by a minimal promoter located downstream of multiple ZF-binding sites. FIG. 1C shows a dose response and time course of expression of the polypeptide when the cells were cultured in the presence of the inducer, β-estradiol, at a range of concentrations. At each concentration, the bars represent, from left to right, culturing in the presence of β-estradiol for 1, 2, 3, or 4 days. The relative fluorescence intensity indicates the amount expression of GFP. The β-estradiol-inducible system described in FIG. 1B was used with the Saccharomyces cerevisiae TEF1 promoter to express the ZF transcription factor and a minimal GAP promoter preceded by nine binding sites of ZF43-8, driving inducible expression of GFP.

FIG. 2 shows that the amount of the polypeptide produced varies depending on the number of transcription factor binding sites upstream of the transgene promoter. The relative fluorescence intensity indicates the amount expression of GFP. A schematic representing the number and spacing of the transcription factor binding sites (triangles) for each of the indicated strains tested is shown below the graph. Different combinations of three transcription factor binding sites were placed approximately 200, 250, or 500 base pairs (bp) from the “ATG” start site, as well as spaced approximately 20 or 40 bp from each other. For each strain, the bar on the left represents expression under “uninduced” conditions in which the strain was cultured in the absence of the inducer. The bar on the right represents expression under “induced” conditions in which the strain was cultured in the presence of the inducer. The data indicate that more transcription factor binding sites resulted in higher expression of the polypeptide (GFP), whereas changing the spacing between the binding sites did not significantly improve levels of expression. Error bars represent s.e.m. (n=3).

FIGS. 3A and 3B show expression of GFP under control of different combinations of promoters regulating expression of the zinc finger transcription factor, transcription factor binding sites, and minimal promoters (inducible promoter) (promoter-ZF-TF/ZF-TF bs-mPromoter), as measured in relative fluorescence units. FIG. 3A shows relative fluorescence units for each of the strains. For each strain, the bar on the left represents expression under “uninduced” conditions in which the strain was cultured in the absence of the inducer. The bar on the right represents expression under “induced” conditions in which the strain was cultured in the presence of the inducer. Error bars represent s.e.m. (n=3). FIG. 3B shows the fold increase in fluorescence intensity for each strain when cultured in the presence of the inducer (ON state) relative to the fluorescence intensity when the strain was cultured in the absence of the inducer (OFF state). Error bars represent s.e.m. (n=3).

FIGS. 4A-4C show polypeptide production from K. phaffi strains containing a β-estradiol-inducible system and a methanol-inducible system. FIG. 4A presents schematics of cells encoding the β-estradiol expression system described herein (regulating GFP expression) and a methanol-inducible expression system comprising the AOX1 promoter (regulating RFP expression). The top left cell is cultured in the presence of glycerol and does not express GFP or RFP. The top right cell is cultured in the presence of glycerol and β-estradiol, which induces expression of GFP. RFP is not expressed. The bottom left cell is cultured in the presence of methanol, which induces expression of RFP under control of PAOX1. GFP is not expressed. The bottom right cell is cultured in the presence of methanol and β-estradiol, resulting in the expression of both RFP and GFP. FIG. 4B shows GFP production by the indicated strains following induction with β-estradiol in BMGY medium, RFP production when induced with BMMY and both GFP and RFP when induced with b-estradiol in BMMY for 24 hours. Error bars represent s.e.m. (n=3). GFP production is shown on the left axis, and RFP production is shown on the right axis. FIG. 4C presents a stained protein gel of the precipitated culture supernatant from the indicated strains 245R, 246R, and 255R cultured for 24 hours in BMGY for β-estradiol (E) or BMMY for methanol (M). HGH and IFNα2b were run as controls for protein size comparison in lanes 8 and 9, respectively, and the approximate size of hGH and IFNα2b are indicated by arrows.

FIGS. 5A-5C show programmable polypeptide production with engineered K. phaffi strain 255B in an integrated, milliliter scale table-top microbioreactor operated continuously for portable manufacturing. FIG. 5A is a schematic showing the microbioreactor. The principal component of the microbioreactor is a polycarbonate-PDMS membrane-polycarbonate sandwiched chip with active microfluidic circuits that are equipped for pneumatic routing of reagents, precise peristaltic injection, growth chamber mixing and fluid extraction. FIG. 5B shows the optical density of a three-day continuous cultivation experiments for selectable production of two polypeptides. The different operational phases are indicated in labeled boxes for one representative experiment. The microbioreactor enabled high-density cell cultures up to a wet-cell weight (WCW) of 356±27 g/L. FIG. 5C shows the concentration of two polypeptides (IFNα2b and rHGH) at the indicated time points, as measured using ELISA. Error bars represent s.e.m. (n=4)

FIGS. 6A and 6B show copy number of the landing pad, IFNα2b-expression construct, and rHGH-expression construct integrated in the genome of the K. phaffi strains described herein. FIG. 6A shows standard curves generated from qPCR amplification of plasmids carrying the landing pad, hGH, or IFNα-2b genes. FIG. 6B presents the crossing point (Cp) values of the landing pad, hGH, and IFNα-2b genes generated from qPCR amplification of 10 ng isolated genomic DNA. Cp values were used to determine absolute copy numbers. The results demonstrate that the copy number was ˜1 in all strains (n=3).

FIG. 7 shows the normalized colony count following integration of plasmids of different sizes into the landing pad containing attB sites for the recombinases BxbI, R4, and TP-901 in the genome of K. phaffi (Integration Site 1). The number of colonies obtained 3 days after transformation by electroporation was approximately constant regardless of plasmid size. Equal moles of plasmid were transformed for each size of plasmid. Error bars represent s.e.m. (n=5)

FIG. 8 shows production of the polypeptide by strains in which the landing pad was integrated into 9 different loci of the K. phaffi genome and is under the control of the β-estradiol-inducible system. For each strain, the bar on the right represents expression under “induced” conditions in which the strain was cultured in the presence of 1 μM β-estradiol, and the bar on the light represents expression under “uninduced” conditions. The fluorescence was measured using flow-cytometry. Single factor ANOVA determined that there are no statistically significant differences between the groups. Error bars represent s.e.m. (n=3).

FIG. 9 show production of GFP by strains engineered to express GFP under control of different constitutive promoters: The top panel shows relative fluorescence intensity from strains expressing GFP under the control of promoter GCW14, S. cerevisiae TEF1 (scTEF1), long and short versions of K. phaffi TEF1 (ppTEF1) and GAP. The bottom panel shows relative fluorescence intensity from strains expressing GFP under the control of promoter the GAP promoter (WT GAP) or variations of the GAP promoter (GAP1-GAP7), in which two TetO sites were introduced at different positions within the GAP promoter. The promoters tested resulted in a broad range of constitutive GFP expression. Error bars represent s.e.m. (n=2).

FIG. 10 shows relative fluorescence intensity indicating GFP expression by K. phaffi strains at 24, 48, and 72 hours. The left column at each time point shows GFP expression from K. phaffi strains that are engineered to express GFP under control of the methanol-inducible AOX1 promoter (strain AOX1-GFP). The right column at each time point shows GFP expression from K. phaffi under control of the synthetic β-estradiol-inducible promoter (strain 255). Expression of GFP was induced after 24 hours of outgrowth with β-estradiol in BMGY medium or with BMMY medium and measured using flow cytometry. Error bars represent s.e.m. (n=3).

FIG. 11 presents a stained protein gel of the precipitated culture supernatant showing rHGH secretion by K. phaffi strains expressing rHGH either under control of the AOX1 promoter (strain AOX1-rHGH) induced by methanol (lanes 3 and 8), or under control of β-estradiol-inducible system induced by β-estradiol (strain 255B; lanes 4 and 9). The cells were induced after 48 hours of outgrowth, and the culture supernatant was collected at 24 hours and 48 hours post-induction. The precipitated supernatants were analyzed by PAGE gel electrophoresis and Coomassie staining. HGH was run as a control for protein size comparison in lanes 2 and 7. The results demonstrate that in this growth condition, both systems result in a comparable amount of rHGH production.

FIG. 12 presents a stained protein gel of the precipitated culture supernatant showing rHGH secretion by K. phaffi strains expressing rHGH either under control of the AOX1 promoter (strain AOX1-rHGH) induced by methanol (lanes 3-5), or under control of the β-estradiol-inducible system induced with β-estradiol (lanes 8-10) in BMGY at low OD with minimal outgrowth. The supernatants were analyzed by PAGE gel electrophoresis and Coomassie staining. HGH was run as a control for protein size comparison in lanes 2 and 7.

FIG. 13 presents a stained protein gel of the culture supernatant where rHGH was secreted into the culture medium. Different media formulations were tested to identify conditions that facilitate not only high levels of expression but also high levels of secretion. Strain 255B was grown for 24 hours and then induced for 24 hours with β-estradiol in BMGY medium with or without the indicated antifoam agents. Addition of each of the antifoam agents L81, P2000, and AF204 to the induction media resulted in enhanced rHGH secretion. The estimated size of hGH is indicated by an arrow.

FIG. 14 shows several real-time conditions of a representative microbioreactor experiment. The top-most graph shows the optical density; second graph shows dissolved oxygen; the third graph shows pH; and the bottom graph shows the temperature.

FIG. 15 shows a time course of protein production for three representative microbioreactor runs. Each run consisted of two independent microbioreactors operating in parallel. The cumulative protein production quantity is presented in Table 7.

FIG. 16 shows a schematic representation of recombination of integration of a gene encoding a polypeptide into a site (“landing pad”) on the genome. A parental strain was generated containing landing pads based on attB sites for the recombinases BxbI, R4, and TP-901.1. This strain can be transformed with a transfer vector containing the desired transgene and the corresponding attP site together with a plasmid encoding the corresponding recombinase. Finally, the excess genetic material is excised using a Flippase recombinase system.

FIG. 17 shows a schematic representation of the production of single biologics or multiple biologics in engineered strains. K. phaffi strains are constructed to contain small-molecule inducible gene expression cassettes integrated into the genome via recombinases. These strains produce combination drugs or multiple biologics concurrently via a consolidated, versatile bioprocessing platform. Following production, drugs or biologics may then be separated prior to administration to one or more subjects.

FIGS. 18A-18G show an exemplary integrated bioprocessing platform for flexible therapeutic protein production. FIG. 18A shows a schematic representation of inducible production of one or two biologics from a dual-biologics production strain. FIG. 18B shows titers of hGH (light gray, left column for each condition) and IFN (dark gray, right column for each condition) in the supernatants of K. phaffi strains under different induction conditions. Values represent mean and s.e.m. (n=3). FIG. 18C shows a Western blot probed with anti-hGH and anti-IFN antibodies. Each lane was loaded with 1 μg pure hGH or IFN or 30 μL supernatant of each sample was loaded in each lane. FIG. 18D presents a Western blot showing the ratio of hGH to IFN in supernatants, which depends on the concentration of estrogen. FIG. 18E shows a schematic representation of post-translational processing of HSA and hGH from an HSA-hGH fusion protein. Golgi-localized TEV protease is expressed from the estrogen-inducible promoter and translocates to the inner Golgi membrane. The HSA-hGH fusion protein is expressed from the methanol-inducible promoter and enters the Golgi after synthesis in the ER. HSA-hGH is cleaved into HSA and hGH by the TEV protease in the Golgi. HSA (black circles), hGH (gray circles), and a small portion of uncleaved HSA-hGH are secreted from the cell. FIG. 18F presents a stained SDS-PAGE gel showing the correct processing of the fusion protein. HSA, hGH, and uncleaved HSA-hGH are labeled with arrows. FIG. 18G presents Western blots with anti-HSA and anti-hGH antibodies. For SDS-PAGE gels and Western blotting, the abbreviations are as follows: E=estrogen induction, M=methanol induction, E+M=estrogen plus methanol induction. Also in the SDS-PAGE gels and Western blots, the boxed lane labels indicate commercial standards while other lane labels without boxes indicate samples obtained under induction with estrogen and/or methanol.

FIGS. 19A-19E show production of mixtures of monoclonal antibodies in K. phaffi. FIG. 19A shows a schematic representation of the effects of the two antibodies on cancer treatment. T cells activated by dendritic cells in the priming phase proliferate to enter the effector phase. The immune checkpoint inhibitor CTLA4 is expressed only in the priming phase, and the immune checkpoint inhibitor PD1 is upregulated in the effector phase but also present in the priming phase of memory T cells. FIG. 19B shows a schematic representation of the production process of the monoclonal antibody mixture. FIG. 19C shows the effect of culture temperature and duration on the expression of the anti-PD1 antibody. Values represent mean and s.e.m. (n=2). FIG. 19D shows a stained SDS-PAGE gel containing 1 μg commercial anti-PD1 antibody and commercial anti-CTLA4 antibody and 10 μL of purified anti-PD1 antibody (“homemade” preparation from K. phaffi), anti-CTLA4 antibody (“homemade” preparation from K. phaffi), and a mixture of both anti-PD 1 and anti-CTLA4 (“homemade” preparation from K. phaffi) were loaded in each lane. FIG. 19E shows the activities of antibody combinations tested in cell-binding assays. Primary T cells were activated and experiments were performed after 3 days (priming phase) and 10 days (effector phase). The first row presents flow cytometry graphs showing verification of the presence of the receptors using labeled commercial anti-PD1 and anti-CTLA4 antibodies or control staining. The second row shows evaluation of the binding of homemade antibodies to activated primary T cells, using labeled anti-human secondary antibodies. The third row shows verification of the binding targets of homemade antibodies by competitive binding assays using commercial antibodies. Values represent mean and s.e.m. (n=2).

FIGS. 20A-20H show simultaneous production of multiple drugs by an integrated co-culture and separation process. FIG. 20A shows a comparison of the total time for drug manufacturing using different strategies. FIG. 20B shows a schematic representation of the co-production of hGH and HSA. FIG. 20C presents a stained SDS-PAGE gel showing analysis of protein expression and purification. Lanes were loaded with 1 μg standard HSA hGH, and the indicated samples. FIG. 20D presents MALDI analysis of HSA (component A, left panel) and hGH (component B, right panel) after purification. FIG. 20E shows a schematic representation of separation of HSA and hGH using Blue Sepharose column, eluting with low salt and high salt eluates. FIG. 20F presents a stained SDS-PAGE gel showing separation of HSA and hGH from the mixed supernatant. Lanes are loaded with 1 μg standard HSA, hGH, or 30 μL of the indicated samples. FIG. 20G shows a schematic representation of the simultaneous production of three biologics by multiplexed co-culture of a dual-biologics strain (producing hGH and HSA) and a single biologic strain (producing an anti-PD1 antibody) and separation using two affinity columns. FIG. 20H presents a stained SDS-PAGE gel showing separation of the mixture of the supernatant consisting of HSA, hGH, and anti-PD1 antibody. Each lane was loaded with 1 μg standard anti-PD1 antibody, HSA, hGH, or 30 μL of the indicated samples. The boxed lane labels indicate commercial standards while other lane labels without boxes denote samples obtained under the indicated conditions.

FIGS. 21A-21H show the development of a triple-biologics production strain of K. phaffi. FIG. 21A presents a schematic representation of an exemplary IPTG-inducible system. This system utilizes the interaction of the lac repressor (Lad) and the lac operator (LacO). Constitutively expressed Lac repressors bind the lac operator, which prevents transcription from the K. phaffi GAP promoter. IPTG interacts with the lac repressor, which releases the latter from the promoter to initiate protein expression. FIG. 21B shows the dose response of GFP expression using the IPTG-inducible system. Maximum fluorescence levels were achieved with 100 mM IPTG at 48 hours. Values represent mean and s.e.m. (n=2). FIG. 21C shows the construction of a strain producing 3 fluorescent proteins: GFP under the control of an estrogen-inducible promoter, RFP under the control of a methanol-inducible promoter, and CFP under the control of an IPTG-inducible promoter. Values represent mean and s.e.m. (n=3). FIG. 21D shows testing of the a strain producing 3 fluorescent proteins of FIG. 21C. The top panel shows GFP fluorescence; the middle panel shows RFP fluorescence; and the bottom panel shows CFP fluorescence. FIG. 21E shows a schematic representation of exemplary inducible promoters and exemplary therapeutic proteins. FIG. 21F presents a stained SDS-PAGE gel showing protein expression under different induction conditions. FIG. 21G. shows a Western blot using antibodies for the three therapeutic proteins. FIG. 21H shows analysis of the content of the indicated therapeutic proteins in each supernatant samples. Protein quantities were calculated by using ImageJ software. For SDS-PAGE analysis and Western blotting, each lane was loaded with 1 μg standard proteins (indicated with boxed labels) or 30 μL supernatants of the indicated samples.

FIGS. 22A-22D show the influence of methanol on estrogen-inducible protein expression. BMGY does not contain methanol, whereas BMMY contains methanol. FIG. 22a is a schematic illustration of the suggested mechanism. FIG. 22B shows that the addition of methanol did not increase estrogen-induced intracellular GFP expression. FIG. 22C shows that the addition of methanol increased estrogen-induced secreted hGH expression. FIG. 22D shows that the addition of methanol increased estrogen-induced secreted G-CSF expression.

FIGS. 23A-23B show that the ratio of two secreted proteins is dependent on the dose of inducers used. FIG. 23A is a schematic illustrating inducible secretion of hGH and G-CSF with a 2-biologics strain (pJC034). FIG. 23B shows that the 2-biologics strain was grown in BMGY for 48 hours and then was induced with BMMY with various concentrations of estrogen for 48 hours. 1 mg hGH or G-CSF standards or 30 mL supernatant of each sample was loaded in each lane. The SDS-PAGE gel was stained using Coomassie Blue. “hGH” or “G-CSF” above the gels indicates commercial standards while other text indicates samples under the induction of methanol and various concentrations of estrogen.

FIG. 24 shows overproduced intracellular TEV protease under the control of estrogen caused cell lysis. The dual-biologics production strain (pJC172) was grown in BMGY for 48 hours, and then was induced with BMMY with various concentrations of estrogen for 48 hours. 1 mg HSA or hGH standards or 30 mL supernatant of each sample was loaded in each lane. The SDS-PAGE gel was stained using Coomassie Blue. “HSA” or “hGH” above the gels indicates commercial standards while other text indicates samples under the induction of methanol and various concentrations of estrogen.

FIGS. 25A-25B show purification of anti-CTLA4 antibodies from cell supernatant. FIG. 25A shows the chromatogram of the purification process using FPLC. Blue line (UV) represents the protein concentration. The peak representing anti-CTLA4 antibody is highlighted in the red circle. FIG. 25B shows the SDS-PAGE gel of the components was stained using Coomassie Blue.

FIGS. 26A-26B show Western blotting characterization of the antibodies produced in K. phaffi (see FIG. 19D). “Anti-PD1” alone and “anti-CTLA4” alone above the gels indicates commercial antibodies while samples with “(homemade)” indicates the “homemade” antibodies that were produced in K. phaffi. FIG. 26A shows a Western blot of the antibodies produced in K. phaffi using an anti-human heavy chain primary antibody. FIG. 26B shows Western blot of the antibodies produced in K. phaffi using an anti-human light chain primary antibody.

FIGS. 27A-27C show that the ratio of HSA and hGH in the supernatants depends on the concentration of estrogen. FIG. 27A shows 1 mg HSA or hGH standards or 30 mL supernatant of each sample was loaded in each lane. The SDS-PAGE gel was stained using Coomassie Blue. FIG. 27B shows a Western blot using an anti-human growth hormone primary antibody. FIG. 27C shows a Western blot using an anti-human serum albumin primary antibody. “HSA” or “hGH” above the gels indicates commercial standards while other text indicates samples under the induction of methanol and various concentrations of estrogen.

FIGS. 28A-28E show RP-HPLC purification and analysis of hGH and HSA produced in K. phaffi, corresponding to FIGS. 4c and 4d. FIG. 28A shows a chromatogram of commercial hGH. FIG. 28B shows a chromatogram of commercial HSA. FIG. 28C shows a chromatogram of the elution fraction after Sepharose Blue column purification. FIG. 28D shows a chromatogram of fraction A from FIG. 28C. FIG. 28E shows a chromatogram of fraction B from FIG. 28C.

FIG. 29 shows SDS-PAGE analysis of the separation of the mixture of the commercial HSA and hGH using Blue Sepharose column. “HSS” and “hGH” above the gel indicates commercial standards while the other text indicates the various fractions obtained during the Blue Sepharose purification process.

FIGS. 30A-30C show exemplary constructs for the expression of monoclonal antibodies (mAb). FIG. 30A shows a schematic representation of a construct for the expression of a monoclonal antibody under control of pAOX1, a methanol-inducible promoter, and the AOX1t terminator from the K. phaffi AOX1 gene. Alpha sig is the alpha-factor secretion signal from Saccharomyces cerevisiae. “VH” and “CH” refer to the variable and constant regions of the heavy chain. “VL” and “CL” refer to the variable and constant regions of the light chain. 2A is the T2A sequence (Szymczak-Workman et al., Cold Spring Harb. Protoc. 2012, 199-204 (2012)) that causes a “ribosome-skip”. FIG. 30B shows a landing-pad integration system in which recombinase attB sites are integrated in the genome at the Trp2 locus. The mAb-containing construct has a corresponding attP site for one of the recombinases. The BxbI recombinase is constitutively expressed from a co-transformed plasmid. BxbI recombines the attB and attP sites resulting in integration of the mAb into the landing pad. FIG. 30C shows a Coomassie-stained Lithium Dodecyl Sulfate-gel (LDS-PAGE) of purified mAbs. Lanes 1, 2 and 3 show anti-Ebola antibodies 2G4, 13C6 and 4G7, respectively; whereas lane 4 is mAb 2G12 (positive control; mAb 2G12; Fraunhofer IME, Aachen, Germany). The 2G4 antibody was produced from a strain generated by recombinase-mediated integration, while the 13C6 and 4G7 antibodies were from strains generated by integration by homologous recombination of linearized plasmid DNA.

FIGS. 31A-31C show representative micrographs of immunofluorescence assays of ZMapp monoclonal antibodies (mAbs). FIG. 31A shows antibody 2G4; FIG. 31B shows antibody 4G7; and FIG. 31C shows antibody 13C6. Cell nuclei are stained with DAPI. For each mAb, cells transfected (“transfected,” left panels) and cells that have not been transfected (“untransfected,” right panels) with pCAGGS-ZEBOV GP1,2 are shown. Also for each mAb, both a fluorescent image (showing GFP only, top panels) and bright-field-DAPI-GFP merge image (bottom panels) are shown. Images were taken at 40× magnification. A 100 μm scale bar is shown in each of the images.

FIG. 32 shows a schematic representation of a rapid development cycle for anti-pathogen monoclonal antibodies produced from glycoengineered K. phaffi (P. pastoris). MBR denotes a microbioreactor capable for localized and rapid production of therapeutic proteins²³.

### DETAILED DESCRIPTION

Conventional methods of engineering cells for the production of desired polypeptides include insertion of nucleic acids encoding the desired polypeptide and any associated regulatory factors into a genetic locus of the cell, for example, by homologous recombination. Although these methods are reliable for simple genetic manipulations, there are many limitations when used for more complex manipulations, such as the potential presence of multiple copies of the inserted nucleic acid, limitations to the size of inserted nucleic acid, and the necessity of maintaining extensive regions of homology with the targeted insertion locus of the cell. The methods and cells provided herein allow for integration of large pieces of nucleic acid (e.g., in excess of 5 kilobases) into a genetic locus of a cell without needing long regions of homology to the targeted insertion locus. Unlike methods for recombinase-mediated cassette exchange, which provide exchange of nucleic acid flanked by two recombination sites with nucleic acid, the methods described herein rely on a single recombination site at the integration locus and a single recombination site on the nucleic acid to be integrated, providing integration rather than exchange of nucleic acid. Furthermore, the methods and cells described herein may be used to produce more than one polypeptide in a switchable/inducible manner, such that the accumulated of biomass generated from outgrowth of the cells may be re-used to produce another polypeptide without the need to regrow cells to production level biomass before inducing expression of the other polypeptide.

The invention described herein is based on the development of methods and cells that allow for rapid production of polypeptides, such as therapeutic molecules, potentially at the point of patient care. Following generation of a genetically modified cell that encodes an inducible system and a recombination site using the provided methods, the cell may be further engineered to produce any desired polypeptide or multiple desired polypeptides produced on programmable cues.

This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

The methods described herein involve generating a cell that expresses one or more polypeptide (e.g., therapeutic molecule) and rely on the rapid, specific, and efficient integration of a gene encoding the polypeptide(s) into the cell. The methods involve preparing or providing a genetically modified cell that encodes an inducible system. As used herein, the term “inducible system” refers to components that, when in the presence of an inducer, results in expression of a gene encoding a polypeptide and subsequent production of the polypeptide. Inducible systems may comprise multiple components, such as a transcription factor, transcription factor binding sites, and one or more promoters, such as a promoter regulating expression of the inducible gene. In some embodiments, the inducible system comprises a transcription factor, at least one transcription factor binding site, an inducible promoter, and a recombination site downstream of the inducible promoter.

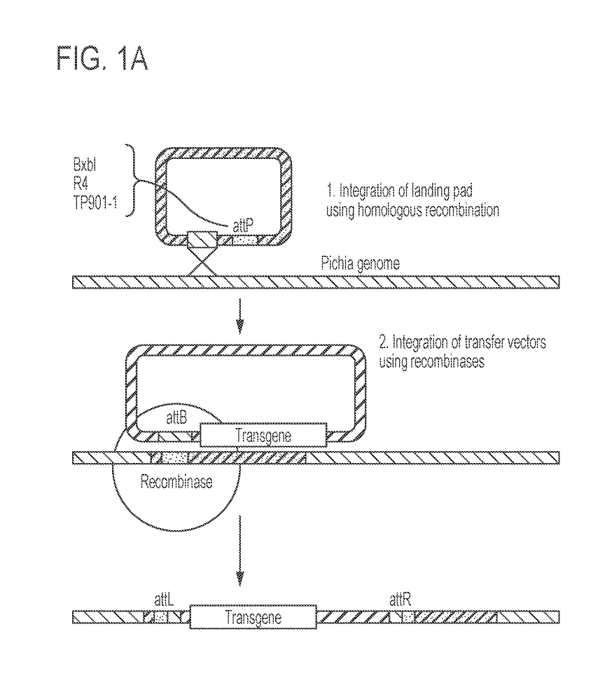

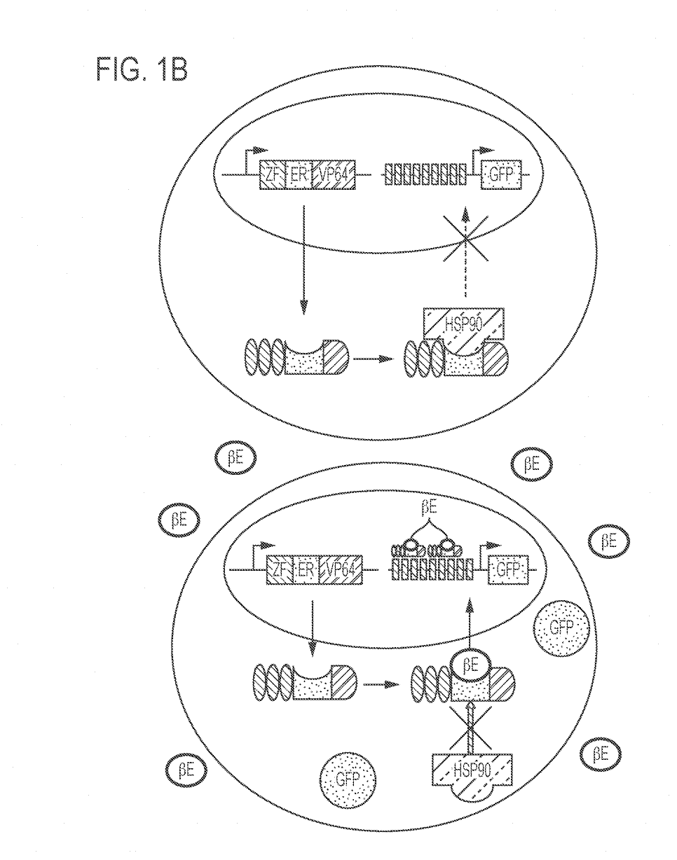

For example as shown in FIG. 1B, in some embodiments, in the absence of the inducer, the transcription factor is maintained in the cytoplasm of the cell. Without wishing to be bound by any particular theory, the transcription factor may be maintained in the cytoplasm by a cytoplasmic factor, such as HSP90. In the presence of the inducer, the transcription factor is able to translocate to the nucleus of the cell, bind to a transcription factor binding site, and induce expression of a gene. In some embodiments, the presence of β-estradiol induces translocation and transcriptional activation of the inducible system.

In general, a transcription factor comprises at least a DNA binding domain that recognizes and binds to a specific nucleic acid sequence upstream of a gene that it regulates. In some embodiments, binding of a transcription factor to a transcription factor binding site functions to recruit transcription machinery (e.g., RNA polymerases) to the promoter of the gene it regulates. In some embodiments, the transcription factor also comprises a transcription activation domain and/or an inducer binding domain. In some embodiments, the transcription factor may be a chimeric transcription factor, comprising components obtained from different sources or proteins. Examples of DNA binding domains include, without limitation, basic helix-loop-helix, basic-leucine zipper, GCC box, helix-turn-helix, serum response factor-like, paired box, winged helix, and zinc finger (ZF) domains. In some embodiments, the DNA binding domain is a ZF domain ZF domains are characterized by the coordination of one or more zinc ions to stabilize the protein fold. ZF domains may be present in many distinct forms including, without limitation, Cys₂-His₂ motif, Cys₂-His-Cys motif, Cys₄ ribbon, Cys₄ GATA family, Cys₆, Cys₈, Cys₃-His-Cys₄ RING Fingers. In some embodiments, the ZF domain is the ZF43-8 DNA binding domain.

Any transcription activation domain known in the art may be compatible with the transcription factors used in the invention described herein. Transcription activation domains may function to activate transcription by interacting with a DNA binding domain and transcriptional machinery (e.g., RNA polymerases). In some embodiments, the transcription activation domain is obtained from a transcription factor. In some embodiments, the transcription activation domain is a synthetic transcription activation domain, for example the VP64 transcription activation domain is a tetramer of tandem copies of the Herpes Simplex virus VP16 transcription activator domain connected with linker peptides. In some embodiments, the transcriptional activation domain is the p65 transcriptional activation domain

Any inducer binding domain known in the art may be compatible with the transcription factors described herein. As used herein, an “inducer binding domain” refers to a domain of the transcription factor that binds a molecule, referred to an inducer, resulting in transcriptional activation and expression of a gene encoding the polypeptide. In some embodiments, in the absence of the inducer, the inducer binding domain of the transcription factor is bound or inactivated by another molecule to maintain the transcription factor in an inactive state thereby preventing expression of the gene encoding the polypeptide. Any protein domain that is able to bind the inducer may be compatible with the inducible system described herein. Examples of inducers and corresponding inducer binding domains will be known in the art and include, without limitation, methanol, IPTG, copper, antibiotics such as tetracycline, carbon source, estrogen (such as β-estradiol), light, and steroids. An example in which the inducer is β-estradiol, the inducer binding domain may be any domain that is able to bind β-estradiol. In some embodiments, the β-estradiol binding domain is obtained from the human estrogen receptor.

The concentration of the inducer to induce transcriptional activation and expression of the gene encoding the polypeptide will depend on factors such as any of the components of the inducible system, the polypeptide to be expressed, and the genetic locus of the inducible system. Optimization of the concentration of the inducer would be considered routine optimization for one of skill in the art. In some embodiments, the concentration of the inducer is between 0.001-50 μM, 0.05-10 μM, 0.01-5 μM, 0.05-1 μM, or 0.1-1 μM. In some embodiments, the concentration of the inducer is at least 0.01 μM, 0.02 μM, 0.03 μM, 0.04 μM, 0.05 μM, 0.06 μM, 0.07 μM, 0.08 μM, 0.09 μM, 0.1 μM. 0.15 μM, 0.2 μM, 0.3 μM, 0.4 μM, 0.5 μM, 0.6 μM, 0.7 μM, 0.8 μM, 0.9 μM, 1.0 μM, 1.1 μM, 1.2 μM, 1.3 μM, 1.4 μM, 1.5 μM or more. In some embodiments, the concentration of the inducer is approximately 0.01 μM. In some embodiments, the concentration of the inducer is approximately 0.1 μM. In some embodiments, the concentration of the inducer is approximately 1 μM.

In some embodiments, the cell encodes more than one inducible systems, e.g. more than one inducible promoters regulating expression of one or more polypeptides. In some embodiments, the cell is exposed to more than one inducer to induce expression of more than one polypeptide. In some embodiments, the cell is exposed to one inducer to induce expression of a polypeptide and then is exposed to one or more additional inducers to induce expression of one or more additional polypeptides.

A transcription factor or components of a transcription factor may be combined to form a chimeric transcription factor may be selected based on a number of factors, such as the affinity of the DNA binding domain for the specific nucleic acid sequence of the transcription factor binding domain. Also within the scope of the present invention are transcription factors or components of transcription factors containing one more mutations relative to a wild-type or naturally occurring transcription factor or component thereof. In some embodiments, one or more mutations may be made in a transcription factor or components of a transcription factor, for example, to modulate (increase or decrease) DNA binding affinity of the transcription factor or component thereof.

In some embodiments, the recombination site and/or the inducible system with the recombination site may be referred to as a “landing pad.” As used herein, a “landing pad” is a region of nucleic acid at a genetic locus of a cell that allows for recombination with another genetic element, such as a plasmid, mediated by a recombinase. The landing pad generally functions as the integration site for the gene encoding the polypeptide, and optionally the corresponding regulatory factors, such as a signal peptide, into the genetic locus of the cell (e.g., the genome of the cell). In some embodiments, the landing pad contains more than one recombination site for the independent integration of more than one gene encoding more than one polypeptide. In some embodiments, the landing pad also contains at least one additional recombination site (e.g., Frt site) that is compatible with a second recombinase. In some embodiments, the landing pad also contains an antibiotic resistance cassette. In some embodiments, the cell contains more than one landing pads located at different genetic loci in the cell.

The inducible system and/or landing pad may be integrated into a genetic locus of the cell by any methods known in the art. In some embodiments, the inducible system is integrated into the genome of a yeast cell by homologous recombination. In such embodiments, a plasmid containing regions of nucleic acid homologous to nucleic acid of the desired integration locus may be provided to the cell. In some embodiments, the integration locus is located on chromosome 2 of a Komagataella phaffi cell. In some embodiments, the locus is the TRP2 locus. In some embodiments, the integration locus is between positions 8346-9028 or positions 1386085-1386686 on chromosome 1 of a Komagataella phaffi cell. In some embodiments, the integration locus is located on chromosome 2 of a Komagataella phaffi cell. In some embodiments, the integration locus is between positions 286540-286072, positions 493919-494400, positions 286989-286140, or positions 808602-809080 on chromosome 2 of a Komagataella phaffi cell. In some embodiments, the integration locus is located on chromosome 3 of a Komagataella phaffi cell. In some embodiments, the integration locus is between positions 292747-293351 or positions 1156771-1157374 on chromosome 3 of a Komagataella phaffi cell. In some embodiments, the integration locus is located on chromosome 4 of a Komagataella phaffi cell. In some embodiments, the integration locus is between positions 1547467-1547086 on chromosome 4 of a Komagataella phaffi cell. The selection of an integration locus may depend on factors such as promoter interference, chromatin structure in the nucleic acid region, and other epigenetic modifications that may influence gene expression.

A genetic element, such as a plasmid, may be provided to a genetically modified cell comprising the inducible system described herein. In some embodiments, more than one genetic element is provided to the genetically modified cells. The plasmid may encode a gene encoding one or more polypeptide, optionally a signal peptide, and a recombination site that is compatible with the recombination site of the genetically modified cell. In some embodiments, more than one plasmid each of which encodes a polypeptide, optionally a signal peptide, and a recombination site, is provided to the genetically modified cells. Recombination between the recombination site in the genome of the cell and the compatible recombination site on the plasmid encoding a gene encoding the polypeptide may be achieved by site-specific recombination. Site-specific recombination involves two recombination sites that are recognized by a compatible enzyme with recombinase activity, referred to herein as a recombinase. As also used herein, the term “compatible” refers to two or more components that are able to function together. For example, the recombination site of the genetically modified cell and the recombination site on the plasmid are compatible if, in the presence of an appropriate recombinase, recombination may occur between the recombination sites. Similarly, a compatible recombinase may also be expressed in the genetically modified cell, such that the recombinase recognizes and promotes recombination between the recombination sites. Recombination between the recombination site in the genome of the cell and the recombination site on the plasmid results in integration of the gene encoding the polypeptide, and optionally the signal peptide, into the genome of the cell. In some embodiments, recombination results in the gene encoding the polypeptide, and optionally the signal peptide, being regulated by the inducible promoter. In some embodiments, accessory factors in addition to a recombinase are also involved in site-specific recombination. In some embodiments, a gene encoding the recombinase is present in the cell or is provided to the cell and expressed from a plasmid.

Recombination sites are generally between about 30-200 nucleotides in length and consist of two regions with partial inverted repeat symmetry that are recognized and bound by the recombinase. Without wishing to be bound by any particular theory, the binding of the recombination sites by the recombinase mediates a crossover between the nucleic acid of the cell and the plasmid for recombination. In some embodiments, the recombination site present in the genome of the cell is distinct from (has a different nucleic acid sequence) but is compatible with the recombination site on the plasmid. In some embodiments, the recombination site in the genome of the cell is an attB recombination site and is compatible with an attP site on the plasmid. In some embodiments, the recombination site in the genome of the cell is an attP recombination site and is compatible with an attB site on the plasmid. Following the recombination reaction, the attB and attP sites form attL and attR sites. In other embodiments, the recombination site present within the genome of the cell has the same nucleic acid sequence and is compatible with the recombination site on the plasmid. In some embodiments, the recombination site present in the genome of the cell is a loxP recombination site and is compatible with a loxP site on the plasmid. In some embodiments, the recombination site present in the genome of the cell is a Frt recombination site and is compatible with a Frt site on the plasmid.

In general, site-specific recombination is mediated by tyrosine recombinases or serine recombinases. Recombinases or genes encoding recombinases can be obtained from a variety of sources including bacteria, yeast, and bacteriophage. Examples of recombinases include, without limitation, BxbI, Cre recombinase, Dre recombinase, Flp recombinase, PhiC31 integrase, TnpX, BxbI recombinase, R4 recombinase, TP901 recombinase, HK022, HP1, gamma delta, ParA, Tn3, Gin, PiggyBac transposase, and lambda integrase.

An example of a “landing pad” encoding recombination sites compatible with the recombinases BxbI, R4, TP901, and Frt at the TRP2 locus of K. phaffi is provided by SEQ ID NO: 1:

| | |
|---|---|
| 1 | CACCATAGCT TCAAAATGTT TCTACTCCTT TTTTACTCTT CCAGATTTTC TCGGACTCCG |
| 61 | CGCATCGCCG TACCACTTCA AAACACCCAA GCACAGCATA CTAAATTTCC CCTCTTTCTT |
| 121 | CCTCTAGGGT GTCGTTAATT ACCCGTACTA AAGGTTTGGA AAAGAAAAAA GAGACCGCCT |
| 181 | CGTTTCTTTT TCTTCGTCGA AAAAGGCAAT AAAAATTTTT ATCACGTTTC TTTTTCTTGA |
| 241 | AAATTTTTTT TTTTGATTTT TTTCTCTTTC GATGACCTCC CATTGATATT TAAGTTAATA |
| 301 | AACGGTCTTC AATTTCTCAA GTTTCAGTTT CATTTTTCTT GTTCTATTAC AACTTTTTTT |
| 361 | ACTTCTTGCT CATTAGAAAG AAAGCATAGC AATCTAATCT AAGGGCGGTG TTGACAATTA |
| 421 | ATCATCGGCA TAGTATATCG GCATAGTATA ATACGACAAG GTGAGGAACT AAACCATGGT |
| 481 | AATGAGCCAT ATTCAACGGG AAACGTCTTG CTCTAGGCCG CGATTAAATT CCAACATGGA |
| 541 | TGCTGATTTA TATGGGTATA AATGGGCTCG CGATAATGTC GGGCAATCAG GTGCGACAAT |
| 601 | CTATCGATTG TATGGGAAGC CCGATGCGCC AGAGTTGTTT CTGAAACATG GCAAAGGTAG |
| 661 | CGTTGCCAAT GATGTTACAG ATGAGATGGT CAGACTAAAC TGGCTGACGG AATTTATGCC |
| 721 | TCTTCCGACC ATCAAGCATT TTATCCGTAC TCCTGATGAT GCATGGTTAC TCACCACTGC |
| 781 | GATCCCCGGG AAAACAGCAT TCCAGGTATT AGAAGAATAT CCTGATTCAG GTGAAAATAT |
| 841 | TGTTGATGCG CTGGCAGTGT TCCTGCGCCG GTTGCATTCG ATTCCTGTTT GTAATTGTCC |
| 901 | TTTTAACAGC GATCGCGTAT TTCGTCTCGC TCAGGCGCAA TCACGAATGA ATAACGGTTT |
| 961 | GGTTGATGCG AGTGATTTTG ATGACGAGCG TAATGGCTGG CCTGTTGAAC AAGTCTGGAA |
| 1021 | AGAAATGCAT AAACTTTTGC CATTCTCACC GGATTCAGTC GTCACTCATG GTGATTTCTC |
| 1081 | ACTTGATAAC CTTATTTTTG ACGAGGGGAA ATTAATAGGT TGTATTGATG TTGGACGAGT |
| 1141 | CGGAATCGCA GACCGATACC AGGATCTTGC CATCCTATGG AACTGCCTCG GTGAGTTTTC |
| 1201 | TCCTTCATTA CAGAAACGGC TTTTTCAAAA ATATGGTATT GATAATCCTG ATATGAATAA |
| 1261 | ATTGCAGTTT CATTTGATGC TCGATGAGTT TTTCTAACAC ATCATGTAAT TAGTTATGTC |
| 1321 | ACGCTTACAT TCACGCCCTC CCCCCACATC CGCTCTAACC GAAAAGGAAG GAGTTAGACA |
| 1381 | ACCTGAAGTC TAGGTCCCTA TTTATTTTTT TATAGTTATG TTAGTATTAA GAACGTTATT |
| 1441 | TATATTTCAA ATTTTTCTTT TTTTTCTGTA CAGACGCGTG TACGCATGTA ACATTATACT |
| 1501 | GAAAACCTTG CTTGAGAAGG TTTTGGGACG CTCGAAGGCT TTAATTTGCA AGCTGGAGAC |
| 1561 | CAACATGTGA GCAAAAGGCC AGCAAAAGGC CAGGAACCGT AAAAAGGCCG CGTTGCTGGC |
| 1621 | GTTTTTCCAT AGGCTCCGCC CCCCTGACGA GCATCACAAA AATCGACGCT CAAGTCAGAG |
| 1681 | GTGGCGAAAC CCGACAGGAC TATAAAGATA CCAGGCGTTT CCCCCTGGAA GCTCCCTCGT |
| 1741 | GCGCTCTCCT GTTCCGACCC TGCCGCTTAC CGGATACCTG TCCGCCTTTC TCCCTTCGGG |
| 1801 | AAGCGTGGCG CTTTCTCATA GCTCACGCTG TAGGTATCTC AGTTCGGTGT AGGTCGTTCG |
| 1861 | CTCCAAGCTG GGCTGTGTGC ACGAACCCCC CGTTCAGCCC GACCGCTGCG CCTTATCCGG |
| 1921 | TAACTATCGT CTTGAGTCCA ACCCGGTAAG ACACGACTTA TCGCCACTGG CAGCAGCCAC |
| 1981 | TGGTAACAGG ATTAGCAGAG CGAGGTATGT AGGCGGTGCT ACAGAGTTCT TGAAGTGGTG |
| 2041 | GCCTAACTAC GGCTACACTA GAAGAACAGT ATTTGGTATC TGCGCTCTGC TGAAGCCAGT |
| 2101 | TACCTTCGGA AAAAGAGTTG GTAGCTCTTG ATCCGGCAAA CAAACCACCG CTGGTAGCGG |
| 2161 | TGGTTTTTTT GTTTGCAAGC AGCAGATTAC GCGCAGAAAA AAAGGATCTC AAGAAGATCC |
| 2221 | TTTGATCTTT TCTACGGGGT CTGACGCTCA GTGGAACGAA AACTCACGTT AAGGGATTTT |
| 2281 | GGTCATGCAT GAGATCAGAT CTGAAGTTCC TATACTTTCT AGAGAATAGG AACTTCAAGC |
| 2341 | TTGTGGAACA TTGAGACCAA ACAAGACTCG CTTCGATGCT TTCAGATCCA TTTTCCCAGC |
| 2401 | AGGTACCGTC TCCGGTGCTC CGAAGGTAAG AGCAATGCAA CTCATAGGAG AATTGGAAGG |
| 2461 | AGAAAAGAGA GGTGTTTATG CGGGGGCCGT AGGACACTGG TCGTACGATG GAAAATCGAT |
| 2521 | GGACACATGT ATTGCCTTAA GAACAATGGT CGTCAAGGAC GGTGTCGCTT ACCTTCAAGC |
| 2581 | CGGAGGTGGA ATTGTCTACG ATTCTGACCC CTATGACGAG TACATCGAAA CCATGAACAA |
| 2641 | AATGAGATCC AACAATAACA CCATCTTGGA GGCTGAGAAA ATCTGGACCG ATAGGTTGGC |
| 2701 | CAGAGACGAG AATCAAAGTG AATCCGAAGA AAACGATCAA TGAACGGAGG ACGTAAGTAG |
| 2761 | GAATTTATGT AATCATGCCA ATACATCTTT AGATTTCTTC CTCTTCTTTT TCATGAGATT |
| 2821 | ATTGGAAACC ACCAGAATCG AATATAAAAG GCGAACACCT TTCCCAATTT TGGTTTCTCC |
| 2881 | TGACCCAAAG ACTTTAAATT TAATTTATTT GTCCCTATTT CAATCAATTG AACAACTATT |
| 2941 | TCGGCTGGAC GGCGACGTAA ACGGCCACAA GTTTATGGCC GTGATGACCT GTGTCTTCGT |
| 3001 | GGTTTGTCTG GTCAACCACC GCGGTCTCAG TGGTGTACGG TACAAACCCA AAGCAGCACG |
| 3061 | ACACGGCAAC TACAAGACCC GCGCCGAGGG CATGTTCCCC AAAGCGATAC CACTTGAAGC |
| 3121 | AGTGGTACTG CTTGTGGGTA CACTCTGCGG GTGTGAAGTT CGAGGGCGAC ACCCTGGTGA |
| 3181 | ACCGCATCGA GCTGAAGGGC ATCTTCCAAC TCGCTTAATT GCGAGTTTTT ATTTCGTTTA |
| 3241 | TTTCAATTAA GGTAACTAAA AAACTCCTTT TACACATGAA GCAGCACGAC TTCTTCAAGT |
| 3301 | CCGCCATGCC CGAAAAACGC CTCTTCAGAG TACAGAAGAT TAAGTGAGAC CTTCGTTTGT |
| 3361 | GCGGATCCCC CACACACCAT AGCTTCAAAA TGTTTCTACT CCTTTTTTAC TCTTCCAGAT |
| 3421 | TTTCTCGGAC TCCGCGCATC GCCGTACCAC TTCAAAACAC CCAAGCACAG CATACTAAAT |
| 3481 | TTCCCCTCTT TCTTCCTCTA GGGTGTCGTT AATTACCCGT ACTAAAGGTT TGGAAAAGAA |
| 3541 | AAAAGAGACC GCCTCGTTTC TTTTTCTTCG TCGAAAAAGG CAATAAAAAT TTTTATCACG |
| 3601 | TTTCTTTTTC TTGAAAATTT TTTTTTTTGA TTTTTTTCTC TTTCGATGAC CTCCCATTGA |
| 3661 | TATTTAAGTT AATAAACGGT CTTCAATTTC TCAAGTTTCA GTTTCATTTT TCTTGTTCTA |
| 3721 | TTACAACTTT TTTTACTTCT TGCTCATTAG AAAGAAAGCA TAGCAATCTA ATCTAAGGGC |
| 3781 | GGTGTTGACA ATTAATCATC GGCATAGTAT ATCGGCATAG TATAATACGA CAAGGTGAGG |
| 3841 | AACTAAACCA TGGTAATGAG CCATATTCAA CGGGAAACGT CTTGCTCTAG GCCGCGATTA |
| 3901 | AATTCCAACA TGGATGCTGA TTTATATGGG TATAAATGGG CTCGCGATAA TGTCGGGCAA |
| 3961 | TCAGGTGCGA CAATCTATCG ATTGTATGGG AAGCCCGATG CGCCAGAGTT GTTTCTGAAA |
| 4021 | CATGGCAAAG GTAGCGTTGC CAATGATGTT ACAGATGAGA TGGTCAGACT AAACTGGCTG |
| 4081 | ACGGAATTTA TGCCTCTTCC GACCATCAAG CATTTTATCC GTACTCCTGA TGATGCATGG |
| 4141 | TTACTCACCA CTGCGATCCC CGGGAAAACA GCATTCCAGG TATTAGAAGA ATATCCTGAT |
| 4201 | TCAGGTGAAA ATATTGTTGA TGCGCTGGCA GTGTTCCTGC GCCGGTTGCA TTCGATTCCT |
| 4261 | GT |

An example of a plasmid encoding a recombinase (BxbI) is provided by SEQ ID NO:2:

| | |
|---|---|
| 1 | GACTCTTCGC GATGTACGGG CCAGATATAC GCGTTGACAT TGATTATTGA CTAGTCCACA |
| 61 | CACCATAGCT TCAAAATGTT TCTACTCCTT TTTTACTCTT CCAGATTTTC TCGGACTCCG |
| 121 | CGCATCGCCG TACCACTTCA AAACACCCAA GCACAGCATA CTAAATTTCC CCTCTTTCTT |
| 181 | CCTCTAGGGT GTCGTTAATT ACCCGTACTA AAGGTTTGGA AAAGAAAAAA GAGACCGCCT |
| 241 | CGTTTCTTTT TCTTCGTCGA AAAAGGCAAT AAAAATTTTT ATCACGTTTC TTTTTCTTGA |
| 301 | AAATTTTTTT TTTTGATTTT TTTCTCTTTC GATGACCTCC CATTGATATT TAAGTTAATA |
| 361 | AACGGTCTTC AATTTCTCAA GTTTCAGTTT CATTTTTCTT GTTCTATTAC AACTTTTTTT |
| 421 | ACTTCTTGCT CATTAGAAAG AAAGCATAGC AATCTAATCT AAGGCTAGCG TTTAAACCAC |
| 481 | CATGAGGGCC CTTGTAGTTA TCAGGTTGAG TAGGGTTACG GATGCAACCA CCAGCCCGGA |
| 541 | GCGCCAACTG GAATCATGTC AGCAGCTTTG TGCGCAGCGC GGCTGGGACG TGGTGGGAGT |
| 601 | GGCGGAGGAC TTGGACGTTA GCGGGGCCGT TGACCCATTT GACCGAAAGC GGAGACCTAA |
| 661 | CCTGGCTCGA TGGCTCGCCT TTGAGGAGCA GCCCTTCGAT GTGATCGTCG CATACAGGGT |
| 721 | CGACAGACTG ACCAGATCCA TTCGCCATCT GCAGCAGCTC GTTCACTGGG CGGAGGACCA |
| 781 | CAAAAAGCTC GTGGTGAGTG CAACAGAAGC CCACTTTGAC ACCACAACAC CCTTCGCAGC |
| 841 | CGTCGTGATC GCTCTGATGG GTACCGTTGC CCAGATGGAA TTGGAGGCAA TCAAGGAGCG |
| 901 | GAACAGATCC GCCGCTCATT TCAATATCCG CGCGGGCAAG TACAGGGGTA GTCTCCCACC |
| 961 | CTGGGGGTAT TTGCCTACCC GGGTGGACGG CGAATGGAGG CTTGTTCCCG ATCCCGTGCA |
| 1021 | GCGAGAGCGA ATACTGGAAG TTTATCATCG AGTCGTGGAT AACCATGAAC CACTCCACCT |
| 1081 | GGTGGCCCAC GACCTTAACC GACGCGGCGT GCTGAGCCCT AAGGACTATT TTGCTCAACT |
| 1141 | TCAGGGAAGA GAGCCACAGG GTAGGGAATG GTCAGCCACA GCTCTCAAGC GGTCTATGAT |
| 1201 | TTCCGAAGCA ATGCTCGGGT ACGCAACACT CAATGGCAAG ACAGTTCGAG ACGACGACGG |
| 1261 | GGCCCCCCTG GTTCGGGCCG AACCCATACT TACCCGCGAA CAACTGGAGG CACTTCGCGC |
| 1321 | GGAACTTGTG AAAACAAGCC GAGCCAAACC CGCAGTGAGC ACCCCATCAC TGCTGCTGAG |
| 1381 | GGTGCTCTTC TGTGCCGTGT GCGGCGAACC AGCATACAAG TTCGCTGGCG GGGGTCGAAA |
| 1441 | ACACCCCCGC TACCGGTGTC GCTCAATGGG TTTTCCAAAG CACTGTGGCA ACGGAACAGT |
| 1501 | TGCAATGGCC GAATGGGACG CTTTTTGTGA AGAACAAGTG CTGGATCTTC TGGGCGACGC |
| 1561 | TGAGAGGCTG GAAAAAGTAT GGGTGGCCGG GAGCGACAGC GCCGTTGAGC TCGCCGAGGT |
| 1621 | GAACGCCGAA TTGGTGGACC TGACGAGTCT CATCGGATCT CCAGCATACC GAGCTGGATC |
| 1681 | CCCCCAGCGA GAGGCTCTGG ACGCTCGGAT AGCCGCCCTG GCAGCAAGGC AGGAGGAGCT |
| 1741 | TGAGGGGTTG GAAGCACGGC CTTCAGGATG GGAATGGCGG GAAACAGGAC AGAGATTTGG |
| 1801 | AGACTGGTGG AGGGAACAGG ATACCGCTGC TAAGAACACT TGGCTCAGGT CCATGAATGT |
| 1861 | TCGACTCACC TTCGACGTGA GGGGTGGGTT GACCCGCACC ATTGATTTCG GGGATCTGCA |
| 1921 | GGAGTATGAA CAGCATCTCC GGCTTGGCTC CGTGGTAGAA AGACTTCATA CAGGCATGTC |
| 1981 | ATGAAGATCT ATTAGTTATG TCACGCTTAC ATTCACGCCC TCCCCCCACA TCCGCTCTAA |
| 2041 | CCGAAAAGGA AGGAGTTAGA CAACCTGAAG TCTAGGTCCC TATTTATTTT TTTATAGTTA |
| 2101 | TGTTAGTATT AAGAACGTTA TTTATATTTC AAATTTTTCT TTTTTTTCTG TACAGACGCG |
| 2161 | TGTACGCATG TAACATTATA CTGAAAACCT TGCTTGAGAA GGTTTTGGGA CGCTCGAAGG |
| 2221 | CTTTAATTTG CAAGCTGGAG ACCAACATGT GAGCAAAAGG CCAGCATCTA GAGGGCCCGT |
| 2281 | TTAAACCCGC TGATCAGCCT CGACTGTGCC TTCTAGTTGC CAGCCATCTG TTGTTTGCCC |
| 2341 | CTCCCCCGTG CCTTCCTTGA CCCTGGAAGG TGCCACTCCC ACTGTCCTTT CCTAATAAAA |
| 2401 | TGAGGAAATT GCATCGCATT GTCTGAGTAG GTGTCATTCT ATTCTGGGGG GTGGGGTGGG |
| 2461 | GCAGGACAGC AAGGGGGAGG ATTGGGAAGA CAATAGCAGG CATGCTGGGG ATGCGGTGGG |
| 2521 | CTCTATGGCT TCTACTGGGC GGTTTTATGG ACAGCAAGCG AACCGGAATT GCCAGCTGGG |
| 2581 | GCGCCCTCTG GTAAGGTTGG GAAGCCCTGC AAAGTAAACT GGATGGCTTT CTCGCCGCCA |
| 2641 | AGGATCTGAT GGCGCAGGGG ATCAAGCTCT GATCAAGAGA CAGGATGAGG ATCGTTTCGC |
| 2701 | ATGATTGAAC AAGATGGATT GCACGCAGGT TCTCCGGCCG CTTGGGTGGA GAGGCTATTC |
| 2761 | GGCTATGACT GGGCACAACA GACAATCGGC TGCTCTGATG CCGCCGTGTT CCGGCTGTCA |
| 2821 | GCGCAGGGGC GCCCGGTTCT TTTTGTCAAG ACCGACCTGT CCGGTGCCCT GAATGAACTG |
| 2881 | CAAGACGAGG CAGCGCGGCT ATCGTGGCTG GCCACGACGG GCGTTCCTTG CGCAGCTGTG |
| 2941 | CTCGACGTTG TCACTGAAGC GGGAAGGGAC TGGCTGCTAT TGGGCGAAGT GCCGGGGCAG |
| 3001 | GATCTCCTGT CATCTCACCT TGCTCCTGCC GAGAAAGTAT CCATCATGGC TGATGCAATG |
| 3061 | CGGCGGCTGC ATACGCTTGA TCCGGCTACC TGCCCATTCG ACCACCAAGC GAAACATCGC |
| 3121 | ATCGAGCGAG CACGTACTCG GATGGAAGCC GGTCTTGTCG ATCAGGATGA TCTGGACGAA |
| 3181 | GAGCATCAGG GGCTCGCGCC AGCCGAACTG TTCGCCAGGC TCAAGGCGAG CATGCCCGAC |
| 3241 | GGCGAGGATC TCGTCGTGAC CCATGGCGAT GCCTGCTTGC CGAATATCAT GGTGGAAAAT |
| 3301 | GGCCGCTTTT CTGGATTCAT CGACTGTGGC CGGCTGGGTG TGGCGGACCG CTATCAGGAC |
| 3361 | ATAGCGTTGG CTACCCGTGA TATTGCTGAA GAGCTTGGCG GCGAATGGGC TGACCGCTTC |
| 3421 | CTCGTGCTTT ACGGTATCGC CGCTCCCGAT TCGCAGCGCA TCGCCTTCTA TCGCCTTCTT |
| 3481 | GACGAGTTCT TCTGAATTAT TAACGCTTAC AATTTCCTGA TGCGGTATTT TCTCCTTACG |
| 3541 | CATCTGTGCG GTATTTCACA CCGCATACAG GTGGCACTTT TCGGGGAAAT GTGCGCGGAA |
| 3601 | CCCCTATTTG TTTATTTTTC TAAATACATT CAAATATGTA TCCGCTCATG AGACAATAAC |
| 3661 | CCTGATAAAT GCTTCAATAA TAGCACGTGC TAAAACTTCA TTTTTAATTT AAAAGGATCT |
| 3721 | AGGTGAAGAT CCTTTTTGAT AATCTCATGA CCAAAATCCC TTAACGTGAG TTTTCGTTCC |
| 3781 | ACTGAGCGTC AGACCCCGTA GAAAAGATCA AAGGATCTTC TTGAGATCCT TTTTTTCTGC |
| 3841 | GCGTAATCTG CTGCTTGCAA ACAAAAAAAC CACCGCTACC AGCGGTGGTT TGTTTGCCGG |
| 3901 | ATCAAGAGCT ACCAACTCTT TTTCCGAAGG TAACTGGCTT CAGCAGAGCG CAGATACCAA |
| 3961 | ATACTGTCCT TCTAGTGTAG CCGTAGTTAG GCCACCACTT CAAGAACTCT GTAGCACCGC |
| 4021 | CTACATACCT CGCTCTGCTA ATCCTGTTAC CAGTGGCTGC TGCCAGTGGC GATAAGTCGT |
| 4081 | GTCTTACCGG GTTGGACTCA AGACGATAGT TACCGGATAA GGCGCAGCGG TCGGGCTGAA |
| 4141 | CGGGGGGTTC GTGCACACAG CCCAGCTTGG AGCGAACGAC CTACACCGAA CTGAGATACC |
| 4201 | TACAGCGTGA GCTATGAGAA AGCGCCACGC TTCCCGAAGG GAGAAAGGCG GACAGGTATC |
| 4261 | CGGTAAGCGG CAGGGTCGGA ACAGGAGAGC GCACGAGGGA GCTTCCAGGG GGAAACGCCT |
| 4321 | GGTATCTTTA TAGTCCTGTC GGGTTTCGCC ACCTCTGACT TGAGCGTCGA TTTTTGTGAT |
| 4381 | GCTCGTCAGG GGGGCGGAGC CTATGGAAAA ACGCCAGCAA CGCGGCCTTT TTACGGTTCC |
| 4441 | TGGGCTTTTG CTGGCCTTTT GCTCACATGT TCTT |

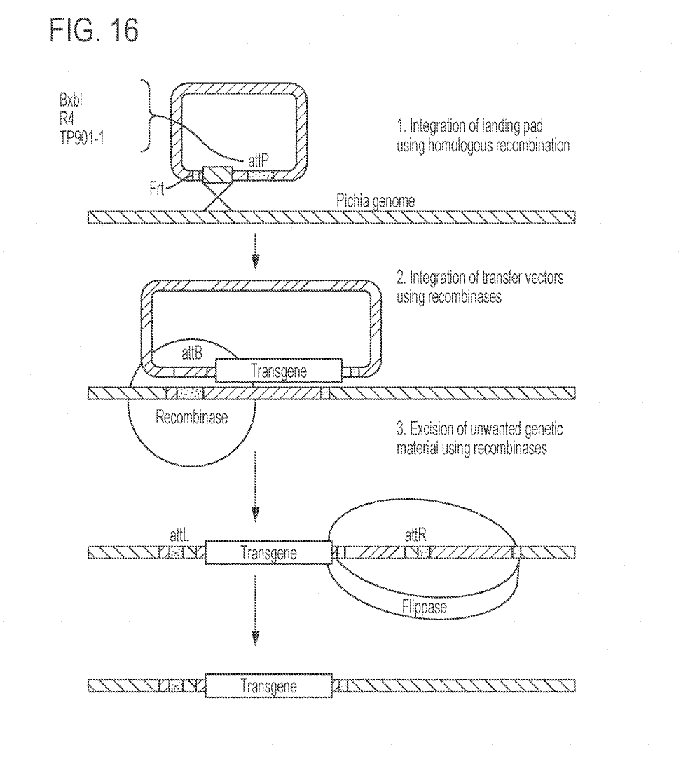

As shown in FIG. 16, in some embodiments, an additional recombination site (e.g., a Frt site) is also integrated into the genome of the cell. The plasmid may further comprise a compatible recombination site (e.g., a second Frt site). Expression of an additional recombinase, such as a flippase, following integration of the gene encoding the polypeptide into the genome may result in excision of excess or undesired genetic material, for example, nucleic acid from the plasmid that is not a part of the gene encoding the polypeptide or a drug resistance or selection cassette. In some embodiments, the additional recombinase is a flippase and recognizes and promotes recombination between two Frt sites.

Aspects of the invention relate to expression of one or more polypeptides, such as one or more therapeutic molecules, in a cell. In some embodiments, the invention relates to expression of one polypeptide by a cell. In some embodiments, the invention relates to expression of more than one polypeptide by the cell. The invention can encompass any cell that recombinantly expresses the genes and an inducible system associated with the invention, including either prokaryotic or eukaryotic cells. Heterologous expression of genes associated with the invention, for production of a polypeptide, such as a therapeutic molecule, is demonstrated in the Examples section using K. phaffi. The novel method for producing polypeptides can also be expressed in other fungi (including other yeast cells), plant cells, mammalian cells, bacterial cells, etc.

In some embodiments the cell is a bacterial cell, such as Escherichia spp., Streptomyces spp., Zymonas spp., Acetobacter spp., Citrobacter spp., Synechocystis spp., Rhizobium spp., Clostridium spp., Corynebacterium spp., Streptococcus spp., Xanthomonas spp., Lactobacillus spp., Lactococcus spp., Bacillus spp., Alcaligenes spp., Pseudomonas spp., Aeromonas spp., Azotobacter spp., Comamonas spp., Mycobacterium spp., Rhodococcus spp., Gluconobacter spp., Ralstonia spp., Acidithiobacillus spp., Microlunatus spp., Geobacter spp., Geobacillus spp., Arthrobacter spp., Flavobacterium spp., Serratia spp., Saccharopolyspora spp., Thermus spp., Stenotrophomonas spp., Chromobacterium spp., Sinorhizobium spp., Saccharopolyspora spp., Agrobacterium spp. and Pantoea spp. The bacterial cell can be a Gram-negative cell such as an Escherichia coli (E. coli) cell, or a Gram-positive cell such as a species of Bacillus.

In other embodiments, the cell is a fungal cell such as a yeast cell, e.g., Saccharomyces spp., Schizosaccharomyces spp., Pichia spp., Komagataella spp., Phaffia spp., Kluyveromyces spp., Candida spp., Talaromyces spp., Brettanomyces spp., Pachysolen spp., Debaryomyces spp., Yarrowia spp., and industrial polyploid yeast strains. Preferably the yeast strain is a Komagataella spp. strain, such as a K. phaffi (P. pastoris) strain. It was recently demonstrated by multigene sequence analysis that strains of Pichia pastoris belong to the species Komagataella phaffi, see for example Kurtzman. J. Ind. Microbiol. Biotechnol. (2009) 36(11):1435-8.

Other examples of fungi include Aspergillus spp., Penicillium spp., Fusarium spp., Rhizopus spp., Acremonium spp., Neurospora spp., Sordaria spp., Magnaporthe spp., Allomyces spp., Ustilago spp., Botrytis spp., and Trichoderma spp.

In other embodiments, the cell is an algal cell, a plant cell, an insect cell, a rodent cell or a mammalian cell, including a human cell.

In some embodiments, one or more of the genes associated with the invention, for example the gene encoding the polypeptide, optionally a signal sequence, and a recombination site are present on a recombinant vector. As used herein, a “vector” may be any of a number of nucleic acids into which a desired sequence or sequences may be inserted by restriction and ligation for transport of the gene between different genetic environments or for expression in a host cell. Vectors are typically composed of DNA, although RNA vectors are also available. Vectors include, but are not limited to: plasmids, fosmids, phagemids, virus genomes, and artificial chromosomes. A vector may be further characterized by one or more endonuclease restriction sites at which the vector may be cut in a determinable fashion and into which a desired DNA sequence may be ligated such that the new recombinant vector retains its ability to replicate in a host cell. In the case of plasmids, replication of the desired sequence may occur many times as the plasmid increases in copy number within a host cell such as a host bacterium or just a single time per host before the host reproduces by mitosis. In the case of phage, replication may occur actively during a lytic phase or passively during a lysogenic phase.

As used herein, a coding sequence (a gene) and regulatory sequences are said to be “operably” joined when they are covalently linked in such a way as to place the expression or transcription of the coding sequence under the influence or control of the regulatory sequences. If it is desired that the coding sequences be translated into a functional protein, two DNA sequences are said to be operably joined if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably joined to a coding sequence if the promoter region were capable of effecting transcription of that DNA sequence such that the resulting transcript can be translated into the desired protein or polypeptide.

When the nucleic acid that encodes any of the genes (e.g. the transcription factor or the polypeptide) of the claimed invention is expressed in a cell, a variety of transcription control sequences (e.g., promoter/enhancer sequences) can be used to direct its expression. The promoter can be a native promoter, i.e., the promoter of the gene in its endogenous context, which provides normal regulation of expression of the gene. In some embodiments the promoter involved in regulating expression of the transcription factor can be a constitutive promoter, i.e., the promoter is unregulated allowing for continual transcription of the transcription factor. Any constitutive promoter known in the art may be compatible with the expression system described herein. In some embodiments, expression of the transcription factor is regulated by a constitutive promoter. Examples of constitutive promoters include, without limitation, ppGVW14, GAP, TEF1, TEF2, ADH1, ADH2, ADH3, ADH4, ADH5, GPD1, GPD2, CYC, STE5, GK1, TDH,3, TP1, HXT7, PGK1, PYK1, and YEF3. Variants of promoters including insertions, deletions, and substitution mutations are also within the scope of the invention described herein. Additional constitutive promoters suitable for use will evident to one of skill in the art and can be found, for example in WO Publication No. 2014/138679, U.S. Pat. No. 8,318,474, and Nacken et al. Gene. 175(1-2):253-260.

Any of a variety of conditional promoters also can be used to regulate expression of the transcription factor, such as promoters controlled by the presence or absence of a molecule, such as an inducible or repressible promoter. In some embodiments, expression of the gene encoding the polypeptide is regulated by an inducible promoter. In some embodiments, the promoter is the AOX1 promoter, the GAP promoter, the TEF1 promoter, a pGCW14 promoter, or a variant thereof.

An example a variant of the TEF1 promoter is the pplongTEF1 promoter provided by SEQ ID NO:4:

TCAGCATCTGGTTACGTAACTCTGGCAACCAGTAACACGCTTAAGGTT
TGGAACAACACTAAACTACCTTGCGGTACTACCATTGACACTACACAT
CCTTAATTCCAATCCTGTCTGGCCTCCTTCACCTTTTAACCATCTTGC
CCATTCCAACTCGTGTCAGATTGCGTATCAAGTGAAAAAAAAAAATTT
TAAAATCTTTAACCCAATCAGGTAATAACTGTCGCCTCTTTTATCTGC
CGCACTGCATGAGGTGTCCCCTTAGTGGGAAAGAGTACTGAGCCAACC
CTGGAGGACAGCAAGGGAAAAATACCTACAACTTGCTTCATAATGGTC
GTAAAAACAATCCTTGTCGGATATAAGTGTTGTAGACTGTCCCTTATC
CTCTGCGATGTTCTTCCTCTCAAAGTTTGCGATTTCTCTCTATCAGAA
TTGCCATCAAGAGACTCAGGACTAATTTCGCAGTCCCACACGCACTCG
TACATGATTGGCTGAAATTTCCCTAAAGAATTTCTTTTTCACGAAAAT
TTTTTTTTACACAAGATTTTCAGCAGATATAAAATGGAGAGCAGGACC
TCCGCTGTGACTCTTCTTTTTTTTCTTTTATTCTCACTACATACATTT
TAGTTATTCGCCAAC

An example a variant of the TEF1 promoter is the ppshortTEF1 promoter provided by SEQ ID NO:5:

ATAACTGTCGCCTCTTTTATCTGCCGCACTGCATGAGGTGTCCCCTTA
GTGGGAAAGAGTACTGAGCCAACCCTGGAGGACAGCAAGGGAAAAATA
CCTACAACTTGCTTCATAATGGTCGTAAAAACAATCCTTGTCGGATAT
AAGTGTTGTAGACTGTCCCTTATCCTCTGCGATGTTCTTCCTCTCAAA
GTTTGCGATTTCTCTCTATCAGAATTGCCATCAAGAGACTCAGGACTA
ATTTCGCAGTCCCACACGCACTCGTACATGATTGGCTGAAATTTCCCT
AAAGAATTTCTTTTTCACGAAAATTTTTTTTTACACAAGATTTTCAGC
AGATATAAAATGGAGAGCAGGACCTCCGCTGTGACTCTTCTTTTTTTT
CTTTTATTCTCACTACATACATTTTAGTTATTCGCCAAC

An example a variant of the TEF1 promoter is the scTEF1 promoter provided by SEQ ID NO:6:

CCACACACCATAGCTTCAAAATGTTTCTACTCCTTTTTTACTCTTCCA
GATTTTCTCGGACTCCGCGCATCGCCGTACCACTTCAAAACACCCAAG
CACAGCATACTAAATTTTCCCTCTTTCTTCCTCTAGGGTGTCGTTAAT
TACCCGTACTAAAGGTTTGGAAAAGAAAAAAGAGACCGCCTCGTTTCT
TTTTCTTCGTCGAAAAAGGCAATAAAAATTTTTATCACGTTTCTTTTT
CTTGAAATTTTTTTTTTTAGTTTTTTTCTCTTTCAGTGACCTCCATTG
ATATTTAAGTTAATAAACGGTCTTCAATTTCTCAAGTTTCAGTTTCAT
TTTTCTTGTTCTATTACAACTTTTTTTACTTCTTGTTCATTAGAAAGA
AAGCATAGCAATCTAATCTAAGG

The sequence of GCW14 promoter is provided by SEQ ID NO: 7:

CAGGTGAACCCACCTAACTATTTTTAACTGGGATCCAGTGAGCTCGCT
GGGTGAAAGCCAACCATCTTTTGTTTCGGGGAACCGTGCTCGCCCCGT
AAAGTTAATTTTTTTTTCCCGCGCAGCTTTAATCTTTCGGCAGAGAAG
GCGTTTTCATCGTAGCGTGGGAACAGAATAATCAGTTCATGTGCTATA
CAGGCACATGGCAGCAGTCACTATTTTGCTTTTTAACCTTAAAGTCGT
TCATCAATCATTAACTGACCAATCAGATTTTTTGCATTTGCCACTTAT
CTAAAAATACTTTTGTATCTCGCAGATACGTTCAGTGGTTTCCAGGAC
AACACCCAAAAAAAGGTATCAATGCCACTAGGCAGTCGGTTTTATTTT
TGGTCACCCACGCAAAGAAGCACCCACCTCTTTTAGGTTTTAAGTTGT
GGGAACAGTAACACCGCCTAGAGCTTCAGGAAAAACCAGTACCTGTGA
CCGCAATTCACCATGATGCAGAATGTTAATTTAAACGAGTGCCAAATC
AAGATTTCAACAGACAAATCAATCGATCCATAGTTACCCATTCCAGCC
TTTTCGTCGTCGAGCCTGCTTCATTCCTGCCTCAGGTGCATAACTTTG
CATGAAAAGTCCAGATTAGGGCAGATTTTGAGTTTAAAATAGGAAATA
TAAACAAATATACCGCGAAAAAGGTTTGTTTATAGCTTTTCGCCTGGT
GCCGTACGGTATAAATACATACTCTCCTCCCCCCCCTGGTTCTCTTTT
TCTTTTGTTACTTACATTTTACCGTTCCGTCACTCGCTTCACTCAACA
ACAAAA

The sequence of GAP promoter is provided by SEQ ID NO: 8:

TCAATTCTTGATTTAGTATACACATAACCAAATTTGGATCAAGTTTGA
AGTAAAACTTTAACTTCAGCTCCTTACATTTGCACTAAGATCTCTGCT
ACTCTGGTCCCAAGTGAACCACCTTTTGGACCCTATTGACCGGACCTT
AACTTGCCAAACCTAAACGCTTAATGCCTCAGACGTTTTAATGCCTCT
CAACACCTCCAAGGTTGCTTTCTTGAGCATGCCTACTAGGAACTTTAA
CGAACTGTGGGGTTGCAGACAGTTTCAGGCGTGTCCCGACCAATATGG
CCTACTAGACTCTCTGAAAAATCACAGTTTTCCAGTAGTTCCGATCAA
ATTACCATCGAAATGGTCCCATAAACGGACATTTGACATCCGTTCCTG
AATTATAGTCTTCCACCGTGGATCATGGTGTTCCTTTTTTTCCCAAAG
AATATCAGCATCCCTTAACTACGTTAGGTCAGTGATGACAATGGACCA
AATTGTTGCAAGGTTTTTCTTTTTCTTTCATCGGCACATTTCAGCCTC
ACATGCGACTATTATCGATCAATGAAATCCATCAAGATTGAAATCTTA
AAATTGCCCCTTTCACTTGACAGGATCCTTTTTTGTAGAAATGTCTTG
GTGTCCTCGTCCAATCAGGTAGCCATCTCTGAAATATCTGGCTCCGTT
GCAACTCCGAACGACCTGCTGGCAACGTAAAATTCTCCGGGGTAAAAC
TTTAATGTGGAGTAATGGAACCAGAAACGTCTCTTCCCTTCTCTCTCC
TTCCACCGCCCGTTACCGTCCCTAGGAAATTTTACTCTGCTGGAGAGC
TTCTTCTACGGCCCCCTTGCAGCAATGCTCTTCCCAGCATTACGTTGC
GGGTAAAACGGAGGTCGTGTACCCGACCTAGCAGCCCAGGGATGGAAA
AGTCCCGGCCGTCGCTGGCAATAATAGCGGGCGGACGCATGTCATGAG
ATTATTGGAAACCACCAGAATCGAATATAAAAGGCGAACACCTTTCCC
AATTTTGGTTTCCCCTGACCCAAAGACTTTAAATTTAATTTATTTGTC
CCTATTTCAATCAATTGAACAACTATCAAAACACA

Examples of variants of the GAP promoter (GAP1-7) are provided by SEQ ID NO: 9-15. Variant 1 (GAP1) is provided by SEQ ID NO: 9:

TCAATTCTTGATTTAGTATACACATAACCAAATTTGGATCAAGTTTGA
AGTAAAACTTTAACTTCAGCTCCTTACATTTGCACTAAGATCTCTGCT
ACTCTGGTCCCAAGTGAACCACCTTTTGGACCCTATTGACCGGACCTT
AACTTGCCAAACCTAAACGCTTAATGCCTCAGACGTTTTAATGCCTCT
CAACACCTCCAAGGTTGCTTTCTTGAGCATGCCTACTAGGAACTTTAA
CGAACTGTGGGGTTGCAGACAGTTTCAGGCGTGTCCCGACCAATATGG
CCTACTAGACTCTCTGAAAAATCACAGTTTTCCAGTAGTTCCGATCAA
ATTACCATCGAAATGGTCCCATAAACGGACATTTGACATCCGTTCCTG
AATTATAGTCTTCCACCGTGGATCATGGTGTTCCTTTTTTTCCCAAAG
AATATCAGCATCCCTTAACTACGTTAGGTCAGTGATGACAATGGACCA
AATTGTTGCAAGGTTTTTCTTTTTCTTTCATCGGCACATTTCAGCCTC
ACATGCGACTATTATCGATCAATGAAATCCATCAAGATTGAAATCTTA
AAATTGCCCCTTTCACTTGACAGGATCCTTTTTTGTAGAAATGTCTTG
GTGTCCTCGTCCAATCAGGTAGCCATCTCTGAAATATCTGGCTCCGTT
GCAACTCCGAACGACCTGCTGGCAACGTAAAATTCTCCGGGGTAAAAC
TTTAATGTGGAGTAATGGAACCAGAAACGTCTCTTCCCTTCTCTCTCC
TTCCACCGCCCGTTACCGTCCCTAGGATCCCTATCAGTGATAGAGATC
TCCCTATCAGTGATAGAGAAATTTTACTCTGCTGGAGAGCTTCTTCTA
CGGCCCCCTTGCAGCAATGCTCTTCCCAGCATTACGTTGCGGGTAAAA
CGGAGGTCGTGTACCCGACCTAGCAGCCCAGGGATGGAAAAGTCCCGG
CCGTCGCTGGCAATAATAGCGGGCGGACGCATGTCATGAGATTATTGG
AAACCACCAGAATCGAATATAAAAGGCGAACACCTTTCCCAATTTTGG
TTTCTCCTGACCCAAAGACTTTAAATTTAATTTATTTGTCCCTATTTC
AATCAATTGAACAACTATCAAAACACA

Variant 2 (GAP2) is provided by SEQ ID NO: 10:

TCAATTCTTGATTTAGTATACACATAACCAAATTTGGATCAAGTTTGA
AGTAAAACTTTAACTTCAGCTCCTTACATTTGCACTAAGATCTCTGCT
ACTCTGGTCCCAAGTGAACCACCTTTTGGACCCTATTGACCGGACCTT
AACTTGCCAAACCTAAACGCTTAATGCCTCAGACGTTTTAATGCCTCT
CAACACCTCCAAGGTTGCTTTCTTGAGCATGCCTACTAGGAACTTTAA
CGAACTGTGGGGTTGCAGACAGTTTCAGGCGTGTCCCGACCAATATGG
CCTACTAGACTCTCTGAAAAATCACAGTTTTCCAGTAGTTCCGATCAA
ATTACCATCGAAATGGTCCCATAAACGGACATTTGACATCCGTTCCTG
AATTATAGTCTTCCACCGTGGATCATGGTGTTCCTTTTTTTCCCAAAG
AATATCAGCATCCCTTAACTACGTTAGGTCAGTGATGACAATGGACCA
AATTGTTGCAAGGTTTTTCTTTTTCTTTCATCGGCACATTTCAGCCTC
ACATGCGACTATTATCGATCAATGAAATCCATCAAGATTGAAATCTTA
AAATTGCCCCTTTCACTTGACAGGATCCTTTTTTGTAGAAATGTCTTG
GTGTCCTCGTCCAATCAGGTAGCCATCTCTGAAATATCTGGCTCCGTT
GCAACTCCGAACGACCTGCTGGCAACGTAAAATTCTCCGGGGTAAAAC
TTTAATGTGGAGTAATGGAACCAGAAACGTCTCTTCCCTTCTCTCTCC
TTCCACCGCCCGTTACCGTCCCTAGGAAATTTTACTCTGCTGGAGAGC
TTCTTCTACGGCCCCCTTGCAGCAATGCTCTCCCTATCAGTGATAGAG
ATCTCCCTATCAGTGATAGAGATTCCCAGCATTACGTTGCGGGTAAAA
CGGAGGTCGTGTACCCGACCTAGCAGCCCAGGGATGGAAAAGTCCCGG
CCGTCGCTGGCAATAATAGCGGGCGGACGCATGTCATGAGATTATTGG
AAACCACCAGAATCGAATATAAAAGGCGAACACCTTTCCCAATTTTGG
TTTCTCCTGACCCAAAGACTTTAAATTTAATTTATTTGTCCCTATTTC
AATCAATTGAACAACTATCAAAACACA

Variant 3 (GAP3) is provided by SEQ ID NO: 11:

TCAATTCTTGATTTAGTATACACATAACCAAATTTGGATCAAGTTTGA
AGTAAAACTTTAACTTCAGCTCCTTACATTTGCACTAAGATCTCTGCT
ACTCTGGTCCCAAGTGAACCACCTTTTGGACCCTATTGACCGGACCTT
AACTTGCCAAACCTAAACGCTTAATGCCTCAGACGTTTTAATGCCTCT
CAACACCTCCAAGGTTGCTTTCTTGAGCATGCCTACTAGGAACTTTAA
CGAACTGTGGGGTTGCAGACAGTTTCAGGCGTGTCCCGACCAATATGG
CCTACTAGACTCTCTGAAAAATCACAGTTTTCCAGTAGTTCCGATCAA
ATTACCATCGAAATGGTCCCATAAACGGACATTTGACATCCGTTCCTG
AATTATAGTCTTCCACCGTGGATCATGGTGTTCCTTTTTTTCCCAAAG
AATATCAGCATCCCTTAACTACGTTAGGTCAGTGATGACAATGGACCA
AATTGTTGCAAGGTTTTTCTTTTTCTTTCATCGGCACATTTCAGCCTC
ACATGCGACTATTATCGATCAATGAAATCCATCAAGATTGAAATCTTA
AAATTGCCCCTTTCACTTGACAGGATCCTTTTTTGTAGAAATGTCTTG
GTGTCCTCGTCCAATCAGGTAGCCATCTCTGAAATATCTGGCTCCGTT
GCAACTCCGAACGACCTGCTGGCAACGTAAAATTCTCCGGGGTAAAAC
TTTAATGTGGAGTAATGGAACCAGAAACGTCTCTTCCCTTCTCTCTCC
TTCCACCGCCCGTTACCGTCCCTAGGAAATTTTACTCTGCTGGAGAGC
TTCTTCTACGGCCCCCTTGCAGCAATGCTCTTCCCAGCATTACGTTGT
CCCTATCAGTGATAGAGATCTCCCTATCAGTGATAGAGACGGGTAAAA
CGGAGGTCGTGTACCCGACCTAGCAGCCCAGGGATGGAAAAGTCCCGG
CCGTCGCTGGCAATAATAGCGGGCGGACGCATGTCATGAGATTATTGG
AAACCACCAGAATCGAATATAAAAGGCGAACACCTTTCCCAATTTTGG
TTTCTCCTGACCCAAAGACTTTAAATTTAATTTATTTGTCCCTATTTC
AATCAATTGAACAACTATCAAAACACA

Variant 4 (GAP4) is provided by SEQ ID NO: 12:

TCAATTCTTGATTTAGTATACACATAACCAAATTTGGATCAAGTTTGA
AGTAAAACTTTAACTTCAGCTCCTTACATTTGCACTAAGATCTCTGCT
ACTCTGGTCCCAAGTGAACCACCTTTTGGACCCTATTGACCGGACCTT
AACTTGCCAAACCTAAACGCTTAATGCCTCAGACGTTTTAATGCCTCT
CAACACCTCCAAGGTTGCTTTCTTGAGCATGCCTACTAGGAACTTTAA
CGAACTGTGGGGTTGCAGACAGTTTCAGGCGTGTCCCGACCAATATGG
CCTACTAGACTCTCTGAAAAATCACAGTTTTCCAGTAGTTCCGATCAA
ATTACCATCGAAATGGTCCCATAAACGGACATTTGACATCCGTTCCTG
AATTATAGTCTTCCACCGTGGATCATGGTGTTCCTTTTTTTCCCAAAG
AATATCAGCATCCCTTAACTACGTTAGGTCAGTGATGACAATGGACCA
AATTGTTGCAAGGTTTTTCTTTTTCTTTCATCGGCACATTTCAGCCTC
ACATGCGACTATTATCGATCAATGAAATCCATCAAGATTGAAATCTTA
AAATTGCCCCTTTCACTTGACAGGATCCTTTTTTGTAGAAATGTCTTG
GTGTCCTCGTCCAATCAGGTAGCCATCTCTGAAATATCTGGCTCCGTT
GCAACTCCGAACGACCTGCTGGCAACGTAAAATTCTCCGGGGTAAAAC
TTTAATGTGGAGTAATGGAACCAGAAACGTCTCTTCCCTTCTCTCTCC
TTCCACCGCCCGTTACCGTCCCTAGGAAATTTTACTCTGCTGGAGAGC
TTCTTCTACGGCCCCCTTGCAGCAATGCTCTTCCCAGCATTACGTTGC
GGGTAAAACGGAGGTCGTGTACCCGACCTAGCAGCCCAGGGATGGAAA
AGTCCCGGCCGTCGCTGGCAATAATAGCGGGCGTCCCTATCAGTGATA
GAGATCTCCCTATCAGTGATAGAGAGACGCATGTCATGAGATTATTGG
AAACCACCAGAATCGAATATAAAAGGCGAACACCTTTCCCAATTTTGG
TTTCTCCTGACCCAAAGACTTTAAATTTAATTTATTTGTCCCTATTTC
AATCAATTGAACAACTATCAAAACACA

Variant 5 (GAP5) is provided by SEQ ID NO: 13:

TCAATTCTTGATTTAGTATACACATAACCAAATTTGGATCAAGTTTGA
AGTAAAACTTTAACTTCAGCTCCTTACATTTGCACTAAGATCTCTGCT
ACTCTGGTCCCAAGTGAACCACCTTTTGGACCCTATTGACCGGACCTT
AACTTGCCAAACCTAAACGCTTAATGCCTCAGACGTTTTAATGCCTCT
CAACACCTCCAAGGTTGCTTTCTTGAGCATGCCTACTAGGAACTTTAA
CGAACTGTGGGGTTGCAGACAGTTTCAGGCGTGTCCCGACCAATATGG
CCTACTAGACTCTCTGAAAAATCACAGTTTTCCAGTAGTTCCGATCAA
ATTACCATCGAAATGGTCCCATAAACGGACATTTGACATCCGTTCCTG
AATTATAGTCTTCCACCGTGGATCATGGTGTTCCTTTTTTTCCCAAAG
AATATCAGCATCCCTTAACTACGTTAGGTCAGTGATGACAATGGACCA
AATTGTTGCAAGGTTTTTCTTTTTCTTTCATCGGCACATTTCAGCCTC
ACATGCGACTATTATCGATCAATGAAATCCATCAAGATTGAAATCTTA
AAATTGCCCCTTTCACTTGACAGGATCCTTTTTTGTAGAAATGTCTTG
GTGTCCTCGTCCAATCAGGTAGCCATCTCTGAAATATCTGGCTCCGTT
GCAACTCCGAACGACCTGCTGGCAACGTAAAATTCTCCGGGGTAAAAC
TTTAATGTGGAGTAATGGAACCAGAAACGTCTCTTCCCTTCTCTCTCC
TTCCACCGCCCGTTACCGTCCCTAGGAAATTTTACTCTGCTGGAGAGC
TTCTTCTACGGCCCCCTTGCAGCAATGCTCTTCCCAGCATTACGTTGC
GGGTAAAACGGAGGTCGTGTACCCGACCTAGCAGCCCAGGGATGGAAA
AGTCCCGGCCGTCGCTGGCAATAATAGCGGGCGGACGCATGTCATGAG
ATTATTGGAAACCACCAGAATCGAATATAAAAGGCGTCCCTATCAGTG
ATAGAGATCTCCCTATCAGTGATAGAGAAACACCTTTCCCAATTTTGG
TTTCTCCTGACCCAAAGACTTTAAATTTAATTTATTTGTCCCTATTTC
AATCAATTGAACAACTATCAAAACACA

Variant 6 (GAP6) is provided by SEQ ID NO: 14:

TCAATTCTTGATTTAGTATACACATAACCAAATTTGGATCAAGTTTGA
AGTAAAACTTTAACTTCAGCTCCTTACATTTGCACTAAGATCTCTGCT
ACTCTGGTCCCAAGTGAACCACCTTTTGGACCCTATTGACCGGACCTT
AACTTGCCAAACCTAAACGCTTAATGCCTCAGACGTTTTAATGCCTCT
CAACACCTCCAAGGTTGCTTTCTTGAGCATGCCTACTAGGAACTTTAA
CGAACTGTGGGGTTGCAGACAGTTTCAGGCGTGTCCCGACCAATATGG
CCTACTAGACTCTCTGAAAAATCACAGTTTTCCAGTAGTTCCGATCAA
ATTACCATCGAAATGGTCCCATAAACGGACATTTGACATCCGTTCCTG
AATTATAGTCTTCCACCGTGGATCATGGTGTTCCTTTTTTTCCCAAAG
AATATCAGCATCCCTTAACTACGTTAGGTCAGTGATGACAATGGACCA
AATTGTTGCAAGGTTTTTCTTTTTCTTTCATCGGCACATTTCAGCCTC
ACATGCGACTATTATCGATCAATGAAATCCATCAAGATTGAAATCTTA
AAATTGCCCCTTTCACTTGACAGGATCCTTTTTTGTAGAAATGTCTTG
GTGTCCTCGTCCAATCAGGTAGCCATCTCTGAAATATCTGGCTCCGTT
GCAACTCCGAACGACCTGCTGGCAACGTAAAATTCTCCGGGGTAAAAC
TTTAATGTGGAGTAATGGAACCAGAAACGTCTCTTCCCTTCTCTCTCC
TTCCACCGCCCGTTACCGTCCCTAGGAAATTTTACTCTGCTGGAGAGC
TTCTTCTACGGCCCCCTTGCAGCAATGCTCTTCCCAGCATTACGTTGC
GGGTAAAACGGAGGTCGTGTACCCGACCTAGCAGCCCAGGGATGGAAA
AGTCCCGGCCGTCGCTGGCAATAATAGCGGGCGGACGCATGTCATGAG
ATTATTGGAAACCACCAGAATCGAATATAAAAGGCGAACACCTTTCCC
AATTTTGGTTTCTCCTGACCCAAAGACTTTAAATTTCCCTATCAGTGA
TAGAGATCTCCCTATCAGTGATAGAGATAATTTATTTGTCCCTATTTC
AATCAATTGAACAACTATCAAAACACA

Variant 7 (GAP7) is provided by SEQ ID NO: 15:

TCAATTCTTGATTTAGTATACACATAACCAAATTTGGATCAAGTTTGA
AGTAAAACTTTAACTTCAGCTCCTTACATTTGCACTAAGATCTCTGCT
ACTCTGGTCCCAAGTGAACCACCTTTTGGACCCTATTGACCGGACCTT
AACTTGCCAAACCTAAACGCTTAATGCCTCAGACGTTTTAATGCCTCT
CAACACCTCCAAGGTTGCTTTCTTGAGCATGCCTACTAGGAACTTTAA
CGAACTGTGGGGTTGCAGACAGTTTCAGGCGTGTCCCGACCAATATGG
CCTACTAGACTCTCTGAAAAATCACAGTTTTCCAGTAGTTCCGATCAA
ATTACCATCGAAATGGTCCCATAAACGGACATTTGACATCCGTTCCTG
AATTATAGTCTTCCACCGTGGATCATGGTGTTCCTTTTTTTCCCAAAG
AATATCAGCATCCCTTAACTACGTTAGGTCAGTGATGACAATGGACCA
AATTGTTGCAAGGTTTTTCTTTTTCTTTCATCGGCACATTTCAGCCTC
ACATGCGACTATTATCGATCAATGAAATCCATCAAGATTGAAATCTTA
AAATTGCCCCTTTCACTTGACAGGATCCTTTTTTGTAGAAATGTCTTG
GTGTCCTCGTCCAATCAGGTAGCCATCTCTGAAATATCTGGCTCCGTT
GCAACTCCGAACGACCTGCTGGCAACGTAAAATTCTCCGGGGTAAAAC
TTTAATGTGGAGTAATGGAACCAGAAACGTCTCTTCCCTTCTCTCTCC
TTCCACCGCCCGTTACCGTCCCTAGGAAATTTTACTCTGCTGGAGAGC
TTCTTCTACGGCCCCCTTGCAGCAATGCTCTTCCCAGCATTACGTTGC
GGGTAAAACGGAGGTCGTGTACCCGACCTAGCAGCCCAGGGATGGAAA
AGTCCCGGCCGTCGCTGGCAATAATAGCGGGCGGACGCATGTCATGAG
ATTATTGGAAACCACCAGAATCGAATATAAAAGGCGAACACCTTTCCC
AATTTTGGTTTCCCCTGACCCAAAGACTTTAAATTTAATTTATTTGTC
CCTATTTCAATCAATTGAACAACTATCAAAACACAGAATTTCCCTATC
AGTGATAGAGATCTCCCTATCAGTGATAGAGAGAATTCATGGTGAGCA
AGG

In some embodiments, a promoter is engineered to be an inducible promoter, for example by the inclusion of one or more transcription factor binding sites upstream of the promoter, such that upon binding of at least one transcription factor binding site with a transcription factor, the promoter is activated and the gene (e.g., the gene encoding the polypeptide) is expressed. In some embodiments, at least 1, 2, 3, 4, 5, 6, 7, 8, 9, 10, 11, 12, 13, 14, or at least 15 transcription factor binding sites are located upstream of the promoter. In some embodiments, one or more transcription factor binding sites are in the plus orientation (on the positive strand of nucleic acid). In other embodiments, one or more transcription factor binding sites are in the negative orientation (on the negative strand of nucleic acid). In some embodiments, more than one transcription factor binding site is present approximately 150-700 base pairs upstream of the promoter. In some embodiments, more than one transcription factor binding site it present upstream of the promoter with approximately 15-50 base pairs between each transcription factor binding site. In some embodiments, there are approximately 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 base pairs between each transcription factor binding site. Selection of the number, spacing, orientation, and strength of the transcription factor binding site(s) may be dependent on factors such amount of polypeptide produced and is within the scope of skill of one in the art.

The additional regulatory sequences may be needed for gene expression and may vary between species or cell types, but shall in general include, as necessary, 5′ non-transcribed and 5′ non-translated sequences involved with the initiation of transcription and translation respectively, such as a TATA box, capping sequence, CAAT sequence, and the like. In particular, such 5′ non-transcribed regulatory sequences will include a promoter region which includes a promoter sequence for transcriptional control of the operably joined gene. Regulatory sequences may also include enhancer sequences or upstream activator sequences as desired.

An example nucleic acid sequence of a genomic region of a genetically modified cell comprising an estrogen inducible system regulating expression of a gene encoding human growth hormone is provided by SEQ ID NO: 3:

| | |
|---|---|
| 1 | GGTCTGACGC TCAGTGGAAC GAAAACTCAC GTTAAGGGAT TTTGGTCATG AGATCAGATC |
| 61 | TGAGCTCGTT TGGCCGTGGC CGTGCTCGTC CTCGTCGGCC GGCTTGTCGA CGACGGCGGT |
| 121 | CTCCGTCGTC AGGATCATCC GGGCCACAAG CTTGCTGACA GAAGCCTCAA GAAAAAAAAA |
| 181 | ATTCTTCTTC GACTATGCTG GAGGCAGAGA TGATCGAGCC GGTAGTTAAC TATATATAGC |
| 241 | TAAATTGGTT CCATCACCTC GAGTCAATTC TTGATTTAGT ATACACATAA CCAAATTTGG |
| 301 | ATCAAGTTTG AAGTAAAACT TTAACTTCAG CTCCTTACAT TTGCACTAAG ATCTCTGCTA |
| 361 | CTCTGGTCCC AAGTGAACCA CCTTTTGGAC CCTATTGACC GGACCTTAAC TTGCCAAACC |
| 421 | TAAACGCTTA ATGCCTCAGA CGTTTTAATG CCTCTCAACA CCTCCAAGGT TGCTTTCTTG |
| 481 | AGCATGCCTA CTAGGAACTT TAACGAACTG TGGGGTTGCA GACAGTTTCA GGCGTGTCCC |
| 541 | GACCAATATG GCCTACTAGA CTCTCTGAAA AATCACAGTT TTCCAGTAGT TCCGATCAAA |
| 601 | TTACCATCGA AATGGTCCCA TAAACGGACA TTTGACATCC GTTCCTGAAT TATAGTCTTC |
| 661 | CACCGTGGAT CATGGTGTTC CTTTTTTTCC CAAAGAATAT CAGCATCCCT TAACTACGTT |
| 721 | AGGTCAGTGA TGACAATGGA CCAAATTGTT GCAAGGTTTT TCTTTTTCTT TCATCGGCAC |
| 781 | ATTTCAGCCT CACATGCGAC TATTATCGAT CAATGAAATC CATCAAGATT GAAATCTTAA |
| 841 | AATTGCCCCT TTCACTTGAC AGGATCCTTT TTTGTAGAAA TGTCTTGGTG TCCTCGTCCA |
| 901 | ATCAGGTAGC CATCTCTGAA ATATCTGGCT CCGTTGCAAC TCCGAACGAC CTGCTGGCAA |
| 961 | CGTAAAATTC TCCGGGGTAA AACTTTAATG TGGAGTAATG GAACCAGAAA CGTCTCTTCC |
| 1021 | CTTCTCTCTC CTTCCACCGC CCGTTACCGT CCCTAGGAAA TTTTACTCTG CTGGAGAGCT |
| 1081 | TCTTCTACGG CCCCCTTGCA GCAATGCTCT TCCCAGCATT ACGTTGCGGG TAAAACGGAG |
| 1141 | GTCGTGTACC CGACCTAGCA GCCCAGGGAT GGAAAAGTCC CGGCCGTCGC TGGCAATAAT |
| 1201 | AGCGGGCGGA CGCATGTCAT GAGATTATTG GAAACCACCA GAATCGAATA TAAAAGGCGA |
| 1261 | ACACCTTTCC CAATTTTGGT TTCCCCTGAC CCAAAGACTT TAAATTTAAT TTATTTGTCC |
| 1321 | CTATTTCAAT CAATTGAACA ACTATCAAAA CACACTAGTA AAAATGCGCG GAGCTCCTAA |
| 1381 | GAAAAAGCGC AAAGTCCGGC CGGCATCTAG ACCCGGGGAG CGCCCCTTCC AGTGTCGCAT |
| 1441 | TTGCATGCGG AACTTTTCGC GCCAGGACAG GCTTGACAGG CATACCCGTA CTCATACCGG |
| 1501 | TGAAAAACCG TTTCAGTGTC GGATCTGTAT GCGAAATTTC TCCCAGAAGG AGCACTTGGC |
| 1561 | GGGGCATCTA CGTACGCACA CCGGCGAGAA GCCATTCCAA TGCCGAATAT GCATGCGCAA |
| 1621 | CTTCAGTCGC CGCGACAACC TGAACCGGCA CCTAAAAACC CACCTGAGGA ACATATGCGG |
| 1681 | CGGAGGCACA CCTGCAGCTG CGTCGACTCT AGAGGATCCA TCTGCTGGAG ACATGAGAGC |
| 1741 | TGCCAACCTT TGGCCAAGCC CGCTCATGAT CAAACGCTCT AAGAAGAACA GCCTGGCCTT |
| 1801 | GTCCCTGACG GCCGACCAGA TGGTCAGTGC CTTGTTGGAT GCTGAGCCCC CCATACTCTA |
| 1861 | TTCCGAGTAT GATCCTACCA GACCCTTCAG TGAAGCTTCG ATGATGGGCT TACTGACCAA |
| 1921 | CCTGGCAGAC AGGGAGCTGG TTCACATGAT CAACTGGGCG AAGAGGGTGC CAGGCTTTGT |
| 1981 | GGATTTGACC CTCCATGATC AGGTCCACCT TCTAGAATGT GCCTGGCTAG AGATCCTGAT |
| 2041 | GATTGGTCTC GTCTGGCGCT CCATGGAGCA CCCAGTGAAG CTACTGTTTG CTCCTAACTT |
| 2101 | GCTCTTGGAC AGGAACCAGG GAAAATGTGT AGAGGGCATG GTGGAGATCT TCGACATGCT |
| 2161 | GCTGGCTACA TCATCTCGGT TCCGCATGAT GAATCTGCAG GGAGAGGAGT TTGTGTGCCT |
| 2221 | CAAATCTATT ATTTTGCTTA ATTCTGGAGT GTACACATTT CTGTCCAGCA CCCTGAAGTC |
| 2281 | TCTGGAAGAG AAGGACCATA TCCACCGAGT CCTGGACAAG ATCACAGACA CTTTGATCCA |
| 2341 | CCTGATGGCC AAGGCAGGCC TGACCCTGCA GCAGCAGCAC CAGCGGCTGG CCCAGCTCCT |
| 2401 | CCTCATCCTC TCCCACATCA GGCACATGAG TAACAAAGGC ATGGAGCATC TGTACAGCAT |
| 2461 | GAAGTGCAAG AACGTGGTGC CCCTCTATGA CCTGCTGCTG GAGATGCTGG ACGCCCACCG |
| 2521 | CCTACATGCG CCCACTAGCC GTGGAGGGGC ATCCGTGGAG GAGACGGACC AAAGCCACTT |
| 2581 | GGCCACTGCG GGCTCTACTT CATCGCCTAG GGCCGACGCG CTGGACGATT TCGATCTCGA |
| 2641 | CATGCTGGGT TCTGATGCCC TCGATGACTT TGACCTGGAT ATGTTGGGAA GCGACGCATT |
| 2701 | GGATGACTTT GATCTGGACA TGCTCGGCTC CGATGCTCTG GACGATTTCG ATCTCGATAT |
| 2761 | GTTAATTAAC TACCCGTACG ACGTTCCGGA CTACGCTTCT TGAGGTACCA TCGGTAGACC |
| 2821 | GGTCTTGCTA GATTCTAATC AAGAGGATGT CAGAATGCCA TTTGCCTGAG AGATGCAGGC |
| 2881 | TTCATTTTTG ATACTTTTTT ATTTGTAACC TATATAGTAT AGGATTTTTT TTGTCATTTT |
| 2941 | GTTTCTTCTC GTACGAGCTT GCTCCTGATC AGCCTATCTC GCAGCTGATG AATATCTTGT |
| 3001 | GGTAGGGGTT TGGGAAAATC ATTCGAGTTT GATGTTTTTC TTGGTATTTC CCACTCCTCT |
| 3061 | TCAGAGTACA GAAGATTAAG TGAGAGCTAG CATATCATAT AGAAGTCATC GCGGCAGATC |
| 3121 | AATTCATCAA TCGGAGTGAG GATATTGCGA GTTTACCACC ATCAATCGGA GTGAGGATCT |
| 3181 | CCAATTGGTG ACGGTCCAGT CATCAATCGG AGTGAGGATT CAGCTGCTTC TCGAGGCCGC |
| 3241 | ACATCAATCG GAGTGAGGAT GTACAGGGTG GGCTGTTCCA CCATCAATCG GAGTGAGGAT |
| 3301 | TTGCGTCAAT GGGGCGGAGT TCATCAATCG GAGTGAGGAT ATCGAAGTCA TCGAGAGCAC |
| 3361 | TCATCAATCG GAGTGAGGAT ATACTCCACC CATTGACGTC ACATCAATCG GAGTGAGGAT |
| 3421 | TGGAACCAGA AGGGTCTCTT CATCAATCGG AGTGAGGATA CCCTATGGGC GCGCCTAACC |
| 3481 | CCTACTTGAC AGCAATATAT AAACAGAAGG AAGCTGCCCT GTCTTAAACC TTTTTTTTTA |
| 3541 | TCATCATTAT TAGCTTACTT TCATAATTGC GACTGGTTCC AATTGACAAG CTTTTGATTT |
| 3601 | TAACGACTTT TAACGACAAC TTGAGAAGAT CAAAAAACAA CTAATTATTC GAAACGCGAA |
| 3661 | TTCATGAGAT TTCCTTCAAT TTTTACTGCT GTTTTATTCG CAGCATCCTC CGCATTAGCT |
| 3721 | GCTCCAGTCA ACACTACAAC AGAAGATGAA ACGGCACAAA TTCCGGCTGA AGCTGTCATC |
| 3781 | GGTTACTCAG ATTTAGAAGG GGATTTCGAT GTTGCTGTTT TGCCATTTTC CAACAGCACA |
| 3841 | AATAACGGGT TATTGTTTAT AAATACTACT ATTGCCAGCA TTGCTGCTAA AGAAGAAGGG |
| 3901 | GTATCTCTCG AGAAGAGATT CCCAACCATT CCCTTATCTA GACTTTTTGA CAACGCTATG |
| 3961 | CTCCGCGCCC ATCGTCTGCA CCAGCTGGCC TTTGACACCT ACCAGGAGTT TGAAGAAGCC |
| 4021 | TATATCCCAA AGGAACAGAA GTATTCATTC CTGCAGAACC CCCAGACCTC CCTCTGTTTC |
| 4081 | TCAGAGTCTA TTCCGACACC CTCCAACAGG GAGGAAACAC AACAGAAATC CAACCTAGAG |
| 4141 | CTGCTCCGCA TCTCCCTGCT GCTCATCCAG TCGTGGCTGG AGCCCGTGCA GTTCCTCAGG |
| 4201 | AGTGTCTTCG CCAACAGCCT GGTGTACGGC GCCTCTGACA GCAACGTCTA TGACCTCCTA |
| 4261 | AAGGACCTAG AGGAAGGCAT CCAAACGCTG ATGGGGAGGC TGGAAGATGG CAGCCCCCGG |
| 4321 | ACTGGGCAGA TCTTCAAGCA GACCTACAGC AAGTTCGACA CAAACTCACA CAACGATGAC |
| 4381 | GCACTACTCA AGAACTACGG GCTGCTCTAC TGCTTCAGGA AGGACATGGA CAAGGTCGAG |
| 4441 | ACATTCCTGC GCATCGTGCA GTGCCGCTCT GTGGAGGGCA GCTGTGGCTT CTAGCGGTCT |
| 4501 | TGCTAGATTC TAATCAAGAG GATGTCAGAA TGCCATTTGC CTGAGAGATG CAGGCTTCAT |
| 4561 | TTTTGATACT TTTTTATTTG TAACCTATAT AGTATAGGAT TTTTTTTGTC ATTTTGTTTC |
| 4621 | TTCTCGTACG AGCTTGCTCC TGATCAGCCT ATCTCGCAGC TGATGAATAT CTTGTGGTAG |
| 4681 | GGGTTTGGGA AAATCATTCG AGTTTGATGT TTTTCTTGGT ATTTCCCACT CCTCTTCAGA |
| 4741 | GTACAGAAGA TTAAGTGAGA CCTTCGTTTG TGCGGATCCC CCACACACCA TAGCTTCAAA |
| 4801 | ATGTTTCTAC TCCTTTTTTA CTCTTCCAGA TTTTCTCGGA CTCCGCGCAT CGCCGTACCA |
| 4861 | CTTCAAAACA CCCAAGCACA GCATACTAAA TTTTCCCTCT TTCTTCCTCT AGGGTGTCGT |
| 4921 | TAATTACCCG TACTAAAGGT TTGGAAAAGA AAAAAGAGAC CGCCTCGTTT CTTTTTCTTC |
| 4981 | GTCGAAAAAG GCAATAAAAA TTTTTATCAC GTTTCTTTTT CTTGAAATTT TTTTTTTTAG |
| 5041 | TTTTTTTCTC TTTCAGTGAC CTCCATTGAT ATTTAAGTTA ATAAACGGTC TTCAATTTCT |
| 5101 | CAAGTTTCAG TTTCATTTTT CTTGTTCTAT TACAACTTTT TTTACTTCTT GTTCATTAGA |
| 5161 | AAGAAAGCAT AGCAATCTAA TCTAAGGGGC GGTGTTGACA ATTAATCATC GGCATAGTAT |
| 5221 | ATCGGCATAG TATAATACGA CAAGGTGAGG AACTAAACCA TGGCCAAGTT GACCAGTGCC |
| 5281 | GTTCCGGTGC TCACCGCGCG CGACGTCGCC GGAGCGGTCG AGTTCTGGAC CGACCGGCTC |
| 5341 | GGGTTCTCCC GGGACTTCGT GGAGGACGAC TTCGCCGGTG TGGTCCGGGA CGACGTGACC |
| 5401 | CTGTTCATCA GCGCGGTCCA GGACCAGGTG GTGCCGGACA ACACCCTGGC CTGGGTGTGG |
| 5461 | GTGCGCGGCC TGGACGAGCT GTACGCCGAG TGGTCGGAGG TCGTGTCCAC GAACTTCCGG |
| 5521 | GACGCCTCCG GGCCGGCCAT GACCGAGATC GGCGAGCAGC CGTGGGGGCG GGAGTTCGCC |
| 5581 | CTGCGCGACC CGGCCGGCAA CTGCGTGCAC TTCGTGGCCG AGGAGCAGGA CTGACACGTC |
| 5641 | CGACGGCGGC CCACGGGTCC CAGGCCTCGG AGATCCGTCC CCCTTTTCCT TTGTCGATAT |
| 5701 | CATGTAATTA GTTATGTCAC GCTTACATTC ACGCCCTCCC CCCACATCCG CTCTAACCGA |
| 5761 | AAAGGAAGGA GTTAGACAAC CTGAAGTCTA GGTCCCTATT TATTTTTTTA TAGTTATGTT |
| 5821 | AGTATTAAGA ACGTTATTTA TATTTCAAAT TTTTCTTTTT TTTCTGTACA GACGCGTGTA |
| 5881 | CGCATGTAAC ATTATACTGA AAACCTTGCT TGAGAAGGTT TTGGGACGCT CGAAGGCTTT |
| 5941 | AATTTGCAAG CTGGAGACCA ACATGTGAGC AAAAGGCCAG CAAAAGGCCA GGAACCGTAA |
| 6001 | AAAGGCCGCG TTGCTGGCGT TTTTCCATAG GCTCCGCCCC CCTGACGAGC ATCACAAAAA |
| 6061 | TCGACGCTCA AGTCAGAGGT GGCGAAACCC GACAGGACTA TAAAGATACC AGGCGTTTCC |
| 6121 | CCCTGGAAGC TCCCTCGTGC GCTCTCCTGT TCCGACCCTG CCGCTTACCG GATACCTGTC |
| 6181 | CGCCTTTCTC CCTTCGGGAA GCGTGGCGCT TTCTCAATGC TCACGCTGTA GGTATCTCAG |
| 6241 | TTCGGTGTAG GTCGTTCGCT CCAAGCTGGG CTGTGTGCAC GAACCCCCCG TTCAGCCCGA |
| 6301 | CCGCTGCGCC TTATCCGGTA ACTATCGTCT TGAGTCCAAC CCGGTAAGAC ACGACTTATC |
| 6361 | GCCACTGGCA GCAGCCACTG GTAACAGGAT TAGCAGAGCG AGGTATGTAG GCGGTGCTAC |
| 6421 | AGAGTTCTTG AAGTGGTGGC CTAACTACGG CTACACTAGA AGGACAGTAT TTGGTATCTG |
| 6481 | CGCTCTGCTG AAGCCAGTTA CCTTCGGAAA AAGAGTTGGT AGCTCTTGAT CCGGCAAACA |
| 6541 | AACCACCGCT GGTAGCGGTG GTTTTTTTGT TTGCAAGCAG CAGATTACGC GCAGAAAAAA |
| 6601 | AGGATCTCAA GAAGATCCTT TGATCTTTTC TACGG |

A nucleic acid molecule, such as a plasmid, that encodes one or more polypeptide associated with the invention can be introduced into a cell or cells using methods and techniques that are standard in the art. For example, nucleic acid molecules can be introduced by standard protocols such as transformation including chemical transformation and electroporation, transduction, particle bombardment, etc. Expressing the nucleic acid molecule encoding the polypeptides of the claimed invention, such as a recombinase, also may be accomplished by integrating the nucleic acid molecule into the genome.

The methods, compositions, and kits described herein allow the production of one or more polypeptides. In some embodiments, the methods, compositions, and kits described herein allow for the production of at least 2, 3, 4, 5, 6, 7, 8, 9, or at least 10 polypeptides from a population of cells. In some embodiments, expression of more than one polypeptide is regulated by more than one inducible system. In some embodiments, expression of more than one polypeptide is regulated by a single inducible system. In some embodiments, the nucleotide sequence encoding a polypeptide may be separated from the nucleotide sequence encoding another polypeptide by a nucleotide sequence that allows for translation of the second polypeptide. In some embodiments, the nucleotide sequence encoding a polypeptide may be separated from the nucleotide sequence encoding another polypeptide by an internal ribosome entry site. In some embodiments, the nucleotide sequence encoding a polypeptide may be separated from the nucleotide sequence encoding another polypeptide by a nucleotide sequence encoding a 2A peptide. In general, 2A peptides are approximately 18-22 amino acids in length and allow for the production of multiple proteins from a single messenger RNA (mRNA). In some embodiments, the 2A peptide is the T2A peptide (EGRGSLLTCGDVEENPGP (SEQ ID NO: 26)), P2A (ATNFSLLKQAGDVEENPGP (SEQ ID NO: 27)), E2A (QCTNYALLKLAGDVESNPGP (SEQ ID NO: 28)), or F2A (VKQTLNFDLLKLAGDVESNPGP (SEQ ID NO: 29).

In some embodiments, the polypeptide may be a therapeutic molecule. As used herein, a “therapeutic molecule” includes any protein that may be administered to a subject and provide a therapeutic effect, such as reduce, alleviate, or eliminate symptoms or pathologies of a disease or disorder. In some embodiments, a therapeutic molecule stimulates or reduces an immune response to an antigen or allergen. Therapeutic molecules include antibodies, such as human or mouse antibodies; hormones, growth factors, fusion proteins, cytokines, chemokines, enzymes, vaccines (antigens), blood factors, thrombolytic agents, interferons, interleukins, that can be used to treat or prevent a disease or disorder. In some embodiments, the therapeutic molecule is glucagon, G-CSF, GM-CSF, Factor IX, Factor VIIa, insulin, agalsidase, dornase alpha, hiruidin, imiglucerase, pleiotrophin, tissue plasminogen activator, or platelet-derived growth factor. In some embodiments, the therapeutic molecule is a vaccine, such as a vaccine against an infectious organism, or component thereof. In some embodiments, the therapeutic molecule is a meningococcal vaccine, a streptococcal vaccine, a malaria vaccine, or a component of a meningococcal vaccine, a streptococcal vaccine, or a malaria vaccine.

The term “antibody” encompasses all forms of antibodies including whole antibodies comprising two light chains and two heavy chains, single chain antibodies (single-chain variable fragments, scFv), dimeric single-chain variable fragments (di-scFv), single domain antibodies (sdAb), Fab fragments, F(ab′)₂, Fab′, Nanobodies®, diabodies, bispecific antibodies, Fc fusion proteins, and chimeric antibodies. Examples of antibodies include antibodies specific for an infectious agent, such as a virus, bacterium, fungi, or prion. In some embodiments, the antibody is a therapeutic monoclonal antibody. Examples of therapeutic monoclonal antibodies include, without limitation, Abagovomab, Abciximab, Actoxumab, Adalimumab, Adecatumumab, Aducanumab, Afelimomab, Afutuzumab, Alacizumab pegol, ALD518, Alemtuzumab, Alirocumab, Altumomab pentetate, Amatuximab, Anatumomab mafenatox, Anifrolumab, Anrukinzumab (=IMA-638), Apolizumab, Arcitumomab, Aselizumab, Atinumab, Atlizumab (=tocilizumab), Atorolimumab, Bapineuzumab, Basiliximab, Bavituximab, Bectumomab, Belimumab, Benralizumab, Bertilimumab, Besilesomab, Bevacizumab, Bezlotoxumab, Biciromab, Biciromab, Bivatuzumab mertansine, Blinatumomab, Blosozumab, Brentuximab vedotin, Briakinumab, Brodalumab, Canakinumab, Cantuzumab mertansine, Cantuzumab ravtansine, Caplacizumab, Capromab pendetide, Carlumab, Catumaxomab, CC49, cBR96-doxorubicin immunoconjugate, Cedelizumab, Certolizumab pegol, Cetuximab, Ch. 14.18, Citatuzumab bogatox, Cixutumumab, Clazakizumab, Clenoliximab, Clivatuzumab tetraxetan, Conatumumab, Concizumab, Crenezumab, CR6261, Dacetuzumab, Daclizumab, Dalotuzumab, Daratumumab, Demcizumab, Denosumab, Detumomab, Dorlimomab aritox, Drozitumab, Duligotumab, Dupilumab, Dusigitumab, Ecromeximab, Eculizumab, Edobacomab, Edrecolomab, Efalizumab, Efungumab, Eldelumab, Elotuzumab, Elsilimomab, Enavatuzumab, Enlimomab pegol, Enokizumab, Enoticumab, Ensituximab, Epitumomab cituxetan, Epratuzumab, Erlizumab, Ertumaxomab, Etaracizumab, Etrolizumab, Evolocumab, Exbivirumab, Fanolesomab, Faralimomab, Farletuzumab, Fasinumab, FBTA05, Felvizumab, Fezakinumab, Ficlatuzumab, Figitumumab, Flanvotumab, Fontolizumab, Foralumab, Foravirumab, Fresolimumab, Fulranumab, Futuximab, Galiximab, Ganitumab, Gantenerumab, Gavilimomab, Gemtuzumab ozogamicin, Gevokizumab, Girentuximab, Glembatumumab vedotin, Golimumab, Gomiliximab, Guselkumab, Ibalizumab, Ibritumomab tiuxetan, Icrucumab, Igovomab, IMAB362, Imciromab, Imgatuzumab, Inclacumab, Indatuximab ravtansine, Infliximab, Intetumumab, Inolimomab, Inotuzumab ozogamicin, Ipilimumab, Iratumumab, Itolizumab, Ixekizumab, Keliximab, Labetuzumab, Lambrolizumab, Lampalizumab, Lebrikizumab, Lemalesomab, Lerdelimumab, Lexatumumab, Libivirumab, Ligelizumab, Lintuzumab, Lirilumab, Lodelcizumab, Lorvotuzumab mertansine, Lucatumumab, Lumiliximab, Mapatumumab, Margetuximab, Maslimomab, Mavrilimumab, Matuzumab, Mepolizumab, Metelimumab, Milatuzumab, Minretumomab, Mitumomab, Mogamulizumab, Morolimumab, Motavizumab, Moxetumomab pasudotox, Muromonab-CD3, Nacolomab tafenatox, Namilumab, Naptumomab estafenatox, Narnatumab, Natalizumab, Nebacumab, Necitumumab, Nerelimomab, Nesvacumab, Nimotuzumab, Nivolumab, Nofetumomab merpentan, Ocaratuzumab, Ocrelizumab, Odulimomab, Ofatumumab, Olaratumab, Olokizumab, Omalizumab, Onartuzumab, Ontuxizumab, Oportuzumab monatox, Oregovomab, Orticumab, Otelixizumab, Otlertuzumab, Oxelumab, Ozanezumab, Ozoralizumab, Pagibaximab, Palivizumab, Panitumumab, Pankomab, Panobacumab, Parsatuzumab, Pascolizumab, Pateclizumab, Patritumab, Pemtumomab, Perakizumab, Pertuzumab, Pexelizumab, Pidilizumab, Pinatuzumab vedotin, Pintumomab, Placulumab, Polatuzumab vedotin, Ponezumab, Priliximab, Pritoxaximab, Pritumumab, PRO 140, Quilizumab, Racotumomab, Radretumab, Rafivirumab, Ramucirumab, Ranibizumab, Raxibacumab, Regavirumab, Reslizumab, Rilotumumab, Rituximab, Robatumumab, Roledumab, Romosozumab, Rontalizumab, Rovelizumab, Ruplizumab, Samalizumab, Sarilumab, Satumomab pendetide, Secukinumab, Seribantumab, Setoxaximab, Sevirumab, Sibrotuzumab, SGN-CD19A, SGN-CD33A, Sifalimumab, Siltuximab, Simtuzumab, Siplizumab, Sirukumab, Solanezumab, Solitomab, Sonepcizumab, Sontuzumab, Stamulumab, Sulesomab, Suvizumab, Tabalumab, Tacatuzumab tetraxetan, Tadocizumab, Talizumab, Tanezumab, Taplitumomab paptox, Tefibazumab, Telimomab aritox, Tenatumomab, Teneliximab, Teplizumab, Teprotumumab, TGN1412, Ticilimumab, Tildrakizumab, Tigatuzumab, TNX-650, Tocilizumab, Toralizumab, Tositumomab, Tovetumab, Tralokinumab, Trastuzumab, TRBS07, Tregalizumab, Tremelimumab, Tucotuzumab celmoleukin, Tuvirumab, Ublituximab, Urelumab, Urtoxazumab, Ustekinumab, Vantictumab, Vapaliximab, Vatelizumab, Vedolizumab, Veltuzumab, Vepalimomab, Vesencumab, Visilizumab, Volociximab, Vorsetuzumab mafodotin, Votumumab, Zalutumumab, Zanolimumab, Zatuximab, Ziralimumab, and Zolimomab.

Examples of hormones include, without limitation, adrenocorticotropic hormone, adiponectin, aldosterone, amylin, androstenedione, angiotensinogen, antidiuretic hormone, antimullerian hormone, atrial natriuretic peptide, brain natriuretic peptide, calcitonin, cholecystokinin, chorionic gonadotropin (CG), corticotrophin, corticotrophin-releasing hormone, cortisol, dihydrotestosterone, dopamine, endothelin, enkephalin, epinephrine, equine chorionic gonadotropin (eCG), erythropoietin, estiol, estradiol, estrone, follicle-stimulating hormone (FSH), galanin, gastrin, ghrelin, glucagon, gonadotropin-releasing hormone, growth hormone (such as human growth hormone, hGH), growth hormone-releasing hormone, histamine, human chorionic gonadotropin (hCG), human placental lactogen, inhibin, insulin, insulin-like growth factor, leptin, leuotrienes, lipotropin, luteinizing hormone (LH), melanocyte stimulating hormone, melatonin, motilin, norepinephrine, orexin, oxytocin, pancreatic polypeptide, parathyroid hormone, progesterone, prolactin, prolactin releasing hormone, prostacyclin, prostaglandins, relaxin, renin, secretin, serotonin, somastostatin, testosterone, thromboxane, thyroid-stimulating hormone (TSH), thyrotropin-releasing hormone, thyroxin.

Examples of cytokines include, without limitation, IL-2, IL-3, IL-4, IL-5, IL-6, IL-7, IL-9, IL-11, IL-13, G-CSF, IL-15, IL-21, GM-CSF, OSM, LIF, IFNγ, IFNα (e.g., IFNα-2a and IFNα-2b), IFNβ (e.g., IFNβ-1a, IFNβ-1b), TNF-α, TNF-β, LT-β, CD40 ligand, Fas ligand, CD27 ligand, CD30 ligand, 4-1BBL, Trail, OPG-L, APRIL, LIGHT, TWEAK, BlyS, IL-10, IL-19, IL-20, IL-22, IL-24, IL26, IL-28a,b, IL-29, IL-12, IL-23, IL-27, TGF-β, IL-la, IL-1β, IL-1 RA, MIF, IL-16, IL-17, IL-18, IL-25.

In some embodiments, the polypeptide is produced and secreted by the cell into the culture medium. Any of the target molecules may comprise a signal sequence or be operably linked to a signal sequence to mediate or enhance secretion of the translated target molecule from the cell into the culture medium. Any signal sequence known in the art that mediates secretion of the target molecule may be compatible for use in the methods described herein. Examples of signal sequences may be obtained from proteins including mating factor alpha-1, alpha factor K, alpha factor T, glycoamylase, inulinase, invertase, lysozyme, serum albumin, alpha-amylase, and killer protein. In some embodiments, the signal sequence is a signal sequence obtained from a yeast protein, such as a Saccharomyces cerevisiae protein. In some embodiments, the signal peptide is obtained from Saccharomyces cerevisiae mating factor alpha-1. Additionally, mutations, substitutions, and truncations of any signal peptide are also within the scope of the present invention. The selection and design, including additional mutations and truncations of a signal peptide are within the ability and discretion of one of ordinary skill in the art.

In other embodiments, the polypeptide may be produced by the cell but not secreted into the culture medium. In such embodiments, the cells may be lysed or dissociated in order to obtain and isolate the polypeptide.

Aspects of the invention relate to methods for producing polypeptides involving culturing any of the cells described herein. Methods of culturing cells, including selection of culture media, culture vessels, and conditions, will be evident to one of ordinary skill in the art. In some embodiments, the cell is a yeast cell. Yeast cells can be cultured in media of any type (rich or minimal). As would be evident to one of skill in the art, routine optimization would allow for use of a variety of types of media. Non-limiting examples of media for cultivating yeast include buffered glycerol-complex (BMGY) media, buffered methanol-complex (BMMY) media, yeast extract peptone dextrose (YPD) broth, yeast extract peptone dextrose adenine (YPAD) broth, yeast nitrogen base (YNB) media, synthetic minimal media, and synthetic complex media.

The selected medium can be supplemented with various additional components. Some non-limiting examples of supplemental components include glucose, xylose, glycerol, methanol, antibiotics, IPTG, amino acids, trace elements, salts, and antifoam agents. The concentration and amount of a supplemental component may be optimized, for example based on production of the target molecule, rate of growth/replication of the cell, or any other factors. It has been found that the addition of an antifoam agent to yeast culture medium may increase the yield of recombinant proteins produced by the cell (e.g., target molecules), see for example Routledge et al. Microb. Cell Fact. 2011 (10)17. Examples of antifoam agents that may be suitable for supplement of the culture media include TERGITOL™ L-81 E, Antifoam A, Antifoam C, Antifoam 204, J673A, polypropylene glycol P2,000 (P2000), or SB212. Additional antifoam agents are known in the art and are commercially available, for example from Sigma-Aldrich.

The cells associated with the invention can be housed in any of the culture vessels known and used in the art. In some embodiments, the cell is cultured in a bioreactor or a shake flask. In some embodiments, the cell is cultured in a microbioreactor, such as a milliliter-scale table top microbioreactor, for small-scale production of a target molecule. In some embodiments, the microbioreactor includes microfluidic chips for culturing the cells and producing the polypeptides described herein. Any of the culturing systems may be a batch culture (batch-fed) or a continuous culture (e.g., perfusion or turbidostat). In some embodiments, the cell is cultured in a microfluidic system. Similarly, other aspects of the medium, and growth conditions of the cells of the invention may be optimized through routine experimentation. For example, pH and temperature are non-limiting examples of such factors.

According to aspects of the invention, one or more polypeptides such as therapeutic molecules are produced through recombinant expression of genes associated with the invention from an inducible expression system. In some embodiments, the polypeptides can be recovered from the cell culture. In some embodiments, the amount of the therapeutic molecule produced is sufficient for a single dose (single therapeutic dose) of the therapeutic molecule for administration to a subject in need. The titer produced of a given polypeptide may be influenced by multiple factors such as choice of media, supplements added to the media, quantity of the inducer, size of the culture, duration of the culturing, and amount of media used. In some embodiments, the total titer of polypeptide is between 1 pg and 10 g. In some embodiments, the total titer of polypeptide is between about 1 pg-1 g, 1 pg-1 mg, 1 pg-1 μg, 1 pg-1 ng, 1 ng-10 g, 1 ng-1 g, 1 ng-1 μg, 1-10 g, 1-1 g, 1-1 mg, 1 mg-10 g, 1 mg-1 g, 5 pg-5 g, 5 pg-500 mg, 5 pg-500 μg, 5 pg-500 ng, 5 ng-5 g, 5 ng-500 mg, 1 ng-500 μg, 5-5 g, 5-500 mg, 50 μg-500 mg, 5 mg-5 g, or about 5 mg-1 g. In some embodiments, the total titer of polypeptide is at least 1, 5, 10, 15, 20, 25, 30, 35, 40, 45, 50, 55, 60, 65, 70, 75, 80, 85, 90, 95, 100, 150 200, 250, 300, 350, 400, 450, 500, 550, 600, 650, 700, 750, 800, 850, 900, 950 or at least 1000 pg. In some embodiments, the total titer of polypeptide is at least 1, 5, 10, 15, 20, 25, 30, 35, 40, 45, 50, 55, 60, 65, 70, 75, 80, 85, 90, 95, 100, 150 200, 250, 300, 350, 400, 450, 500, 550, 600, 650, 700, 750, 800, 850, 900, 950 or at least 1000 ng. In some embodiments, the total titer of polypeptide is at least 5, 6, 7, 8, 9, 10, 15, 20, 25, 30, 35, 40, 45, 50, 55, 60, 65, 70, 75, 80, 85, 90, 95, 100, 150 200, 250, 300, 350, 400, 450, 500, 550, 600, 650, 700, 750, 800, 850, 900, 950 or at least 1000 μg. In some embodiments, the total titer of polypeptide is at least 1, 5, 10, 15, 20, 25, 30, 35, 40, 45, 50, 55, 60, 65, 70, 75, 80, 85, 90, 95, 100, 150 200, 250, 300, 350, 400, 450, 500, 550, 600, 650, 700, 750, 800, 850, 900, 950 or at least 1000 mg. In some embodiments, the total titer of polypeptide is at least 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, or at least 10.0 g/L.

In some embodiments, the cells have been genetically modified such that the cells express more than one polypeptide. For example, the cells may be exposed to a first inducer that induces expression of a polypeptide and be exposed to a second inducer that induces expression of another polypeptide. The total titer of polypeptide produced by the cells may be the total titer of one polypeptide produced or the total titer (the sum) of each of the polypeptides.

In some embodiments, the polypeptide is produced in less than 24 hours of culturing any of the cells described herein. In some embodiments, a titer of at least 5 μg of the polypeptide is produced in less than approximately 24 hours. In some embodiments, at least 5, 6, 7, 8, 9, 10, 15, 20, 25, 30, 35, 40, 45, 50, 55, 60, 65, 70, 75, 80, 85, 90, 95, or at least 100 μg of human growth hormone is produced in approximately 24 hours or less than 24 hours. In some embodiments, at least 5, 6, 7, 8, 9, 10, 15, 20, 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950 or at least 1000 μg of IFNα is produced in approximately 24 hours or less than 24 hours. In some embodiments, at least 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, or at least 10.0 g/L of polypeptide is produced in approximately 24 hours.

In some embodiments, the cells are cultured for approximately 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, or approximately 72 hours. In some embodiments, the cells are cultured for less than about 24 hours. In some embodiments, the cells are cultured for less than about 48 hours. In some embodiments, the cells are culture for less than about 72 hours. In some embodiments, the cells are cultured for more than 72 hours.

In some embodiments, the method further comprising isolating or purifying the polypeptide from the cell culture. In some embodiments, the polypeptide is isolated from the supernatant or cell culture medium. In some embodiments, a pharmaceutically acceptable excipient or carrier suitable for administration to a subject is added to the isolate or purified polypeptide.

Any of the polypeptides produced by the methods and cell described herein may be used in a method of treating a subject. In such embodiments, the polypeptide may be administered to a subject having, suspected of having, or at risk of having a disease or disorder in an effective amount. As used herein, the term “effective amount” refers to any amount of the polypeptide that has a beneficial or therapeutic effect, such as reducing pathologies or symptoms, curing, ameliorating, or maintaining a cure (i.e., preventing relapse) of the disease or disorder. In some embodiments, an effective amount inhibits formation, progression, growth and/or spread (e.g., metastasis). A subject may be a mammal, including but not limited to a dog, cat, horse, cow, pig, sheep, goat, chicken, rodent, or primate. In some embodiments, the subject is a human subject. The human subject may be a pediatric or adult subject.

Also within the scope of the invention described herein are kits that may comprise a genetically modified cell comprising a transcription factor, at least one transcription factor binding site, an inducible promoter, and a first recombination site downstream of and operably linked to the inducible promoter; a recombinase; and a plasmid containing encoding a gene encoding the polypeptide (e.g., a therapeutic molecule), optionally a signal peptide, and a recombination site. The kits can include one or more containers comprising one or more of the components (e.g., genetically modified cell, recombinase, plasmid) described herein. In some embodiments, the genetically modified cell encodes the recombinase. In some embodiments, the kit further comprises a second plasmid encoding the recombinase. The kit may further comprise additional reagents such as buffers, salts, and the like. In some embodiments, the kit further comprises one or more medium, such as a reconstitution medium, an outgrowth medium, and/or a protein production medium. In some embodiments, the cells of the kit are provided in lyophilized form.

In some embodiments, the kit can comprise instructions for use according to any of the methods described herein.

### EXAMPLES

### Example 1: Development of Synthetic Biology and Microbioreactor Platforms for Programmable Production of Biologics at the Point-of-Care

To address this demand for personalized biomanufacturing technologies, a new platform was developed for flexible and portable production of biologic therapeutics at the point of patient care, in short time frames and with limited system requirements. The system described herein allows for the production of near-single-dose levels of polypeptides, such as recombinant human growth hormone (rHGH) and interferon-α2b (IFNα2b) in approximately 24 hours.

### Host Cell Selection

Recombinant biologics for therapeutic use in humans can be produced using a variety of host organisms, including bacteria, yeast, plants, insect cells and mammalian cells (Berlec et al. J. Ind. Microbiol. Biotechnol. (2013) 40, 257-274). The specific host used can have an impact on yields, need for viral inactivation, downstream purification requirements, as well as final product formulation. Mammalian Chinese Hamster Ovary (CHO) cells are the most commonly used host for producing Food and Drug Administration (FDA)-approved biologics

(Zhu, Biotechnol. Adv. (2012) 30, 1158-1170), but they have complex media requirements and their storage requires cryopreservation. Therefore, the long time needed to go from inoculation of biologic-producing CHO cells to release of a drug product, which meets established quality standards, and an FDA-approved safety profile is incompatible with a rapid production system. Yeast are attractive alternatives to CHO cells, as they have simple media requirements, grow quickly to high densities and can be stored as lyophilized material (Berlec et al. J. Ind. Microbiol. Biotechnol. (2013) 40, 257-274; Mattanovich et al. Methods Mol. Biol. (2012) 824, 329-358). Komagataella phaffi (formerly known as Pichia pastoris) is becoming increasingly popular for biologic production, as it (1) can grow to very high densities on simple and inexpensive carbon sources; (2) has a strong yet tightly controlled alcohol oxidase 1 (AOX1) promoter, which can be induced by methanol for high level protein production (up to 10 g/L) and is effectively repressed by glycerol or glucose; (3) is capable of human-like posttranslational modifications, including glycosylation (Vervecken et al. Appl. Environ. Microbiol. (2004) 70, 2639-2646; Vervecken et al. Methods Mol. Biol. (2007) 389, 119-138; Zhang et al. MAbs (2011) 3, 289-298); and (4) secretes heterologous proteins into the extracellular space very efficiently with minimal host protein contamination, thus requiring relatively simple downstream purification systems (Macauley-Patrick et al. Yeast (2005) 22, 249-270; Vogl et al. Curr. Opin. Biotechnol. (2013) 24, 1094-1101).

To date, more than 500 different proteins, including simple peptides, enzymes, hormones, monoclonal antibodies and FDA-approved therapeutics have been expressed in K. phaffi (Thiel Nat. Biotechnol. (2004) 22, 1365-1372; Farid et al. MAbs (2013) 6, 1357-1361; Hwang. PLoS ONE (2013) 8, e71966). Thus, as a proof-of-concept of flexible polypeptide manufacturing using a single host, K. phaffi strains were developed to support the independently selectable production of two different polypeptides (e.g., therapeutics). The use of individual strains that support production of multiple biologics provides significant advantages over the use of multiple strains that each produce single biologics. First, leveraging strains that produce multiple biologics for rapid production of dosage-level biologics enables the re-use of accumulated biomass from the outgrowth period. This approach dramatically improves production speed by avoiding the need to regrow different strains into production-level biomass. Second, for production of multicomponent products, the FDA requires approval of multiple manufacturing lines that each produce individual components and thus a single multiplexed expression platform could offer a potential regulatory advantage.

The AOX1 promoter (PAOX1) is useful for producing one protein on-demand Only a few alternative inducible promoters have been characterized in K. phaffi, including the CUP1, G1 or FLD1 promoters. However, it remains unclear whether these promoters support high levels of expression, while remaining orthogonal to PAOX1 (Prielhofer et al. Microb. Cell Fact. (2013) 12, 5; Koller et al. Yeast (2000) 16, 651-656; Resina et al. Biotechnol. Bioeng. (2005) 91, 760-767). Thus, to enable selectable bioproduction, new inducible promoters were developed and described herein that are orthogonal to and can surpass PAOX1 in promoter strength.

### Transformation Platform

Current transformation methods for K. phaffi rely on genomic integration of small linearized plasmids through homologous recombination, followed by antibiotic selection and screening for high-copy-number integrants (Cereghino et al. 1-BMS Microbiol. Rev. (2000) 24, 45-66). Although these approaches are adequate for simple genetic manipulations, such as introducing small expression cassettes, they have several limitations for more sophisticated synthetic biology applications. In addition, multiple random integration events are undesirable when attempting to compare expression levels between different genetic constructs.

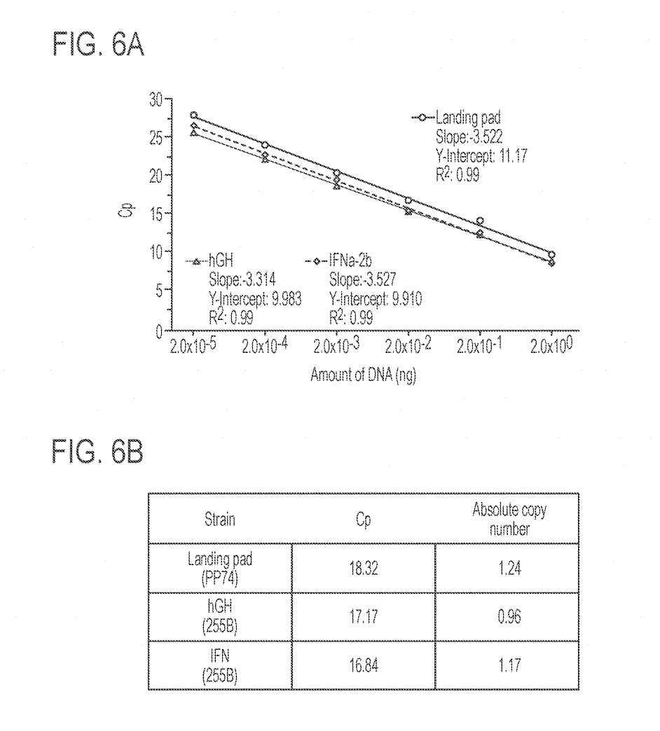

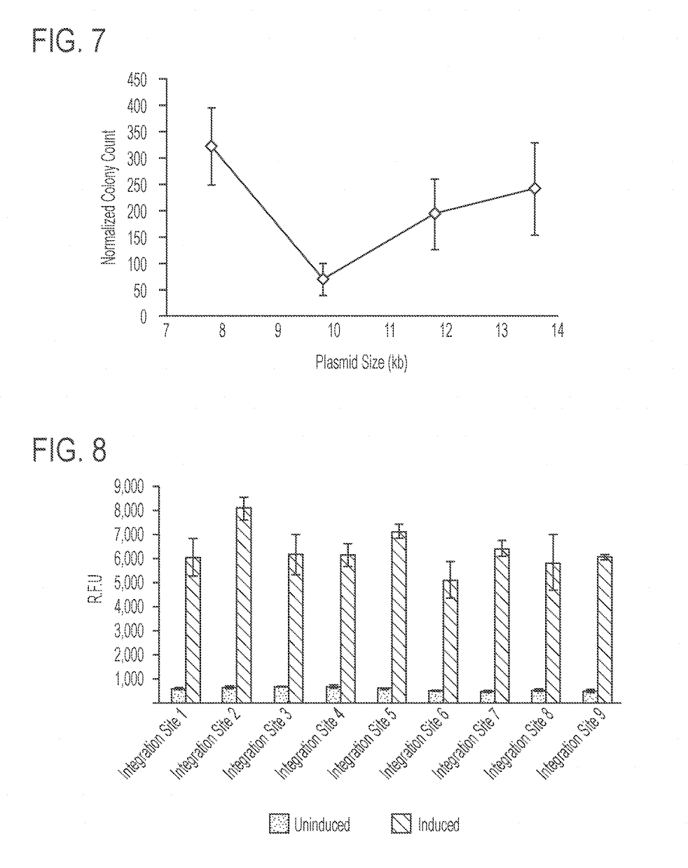

To accelerate the rapid design-test-and-optimize cycle for creating new promoters, a recombinase-based system was developed for the single copy integration of plasmids at a defined loci that is suitable even for large DNA constructs (FIG. 1A). This approach aimed to overcome the rate limiting step of plasmid transformation and genomic integration of synthetic constructs into K. phaffi. First, a parent K. phaffi strain was generated containing attB sites for the recombinases BxbI, R4 and TP-901 (Yamaguchi et al. PLoS ONE (2011) 6, e17267). This was accomplished by traditional integration of a construct containing regions of homology to the Trp2 locus in the K. phaffi genome (Tables 4 and 5, Integration Site 1), attB sites and a kanamycin resistance (KanR) selection cassette. Integration at the Trp2 locus was validated using PCR and a copy number of 1 was verified using quantitative PCR (qPCR; FIGS. 6A and 6B). Then a plasmid for the transient expression of BxbI, R4 or TP901, was co-transformed together with a transfer plasmid containing attP sites for the corresponding recombinase and engineered genetic constructs of interest. This method resulted in ˜50-300 transformants per reaction for DNA constructs ranging from ˜7.8 to 13.6 kb (FIG. 7).

### Optimization of β-Estradiol-Inducible Expression Systems

Using this integration strategy, an inducible transcriptional system was developed consisting of a constitutively expressed zinc-finger (ZF) DNA-binding domain (Khalil et al. Cell (2012) 150, 647-658) fused with the β-estradiol binding domain of the human estrogen receptor, which is coupled with a transcriptional activation domain (Mclsaac et al. Nucleic Acids Res. (2013) 41, e57). At steady state, this synthetic ZF transcription factor (ZF-TF) is sequestered in the cytoplasm by HSP90. Addition of β-estradiol displaces HSP90 and permits translocation of the ZF-TF into the nucleus, where it activates expression of genes regulated by a minimal promoter placed downstream of multiple ZF-binding sites (FIG. 1B). This system offers a highly flexible architecture that can be tuned by modifying different parameters, including the following: (1) affinity of the DNA-binding domain; (2) strength of the transcriptional activation domain; (3) number of binding sites for the ZF; (4) promoter driving expression of the ZF; (5) minimal promoter driving expression of the output; (6) dose of inducer; and (7) integration site.

Initial tuning of expression levels was performed with green fluorescent protein (GFP) as the output quantified using flow cytometry. The ZF DNA-binding domain used in these experiments was ZF43-8 (Khalil et al. Cell (2012) 150, 647-658). The ZF DNA-binding domain and β-estradiol-binding domain of the human estrogen receptor to the VP64 transcriptional activation domain, which has been previously shown to mediate higher levels of expression in mammalian cells than other domains, such as p65 or VP16 (Perez-Pinera et al. Nat. Methods (2013) 10, 239-242). The Saccharomyces cerevisiae TEF1 promoter was used to express the ZF-TF and a minimal GAP promoter preceded by nine binding sites for ZF43-8 was used to drive the inducible expression of GFP. At 24 hours, dose-response curves showed that maximum expression of GFP could be attained with only 0.01 μM β-estradiol (FIG. 1C). However, at 48 h, 0.1-1 μM β-estradiol was necessary to fully saturate this system.

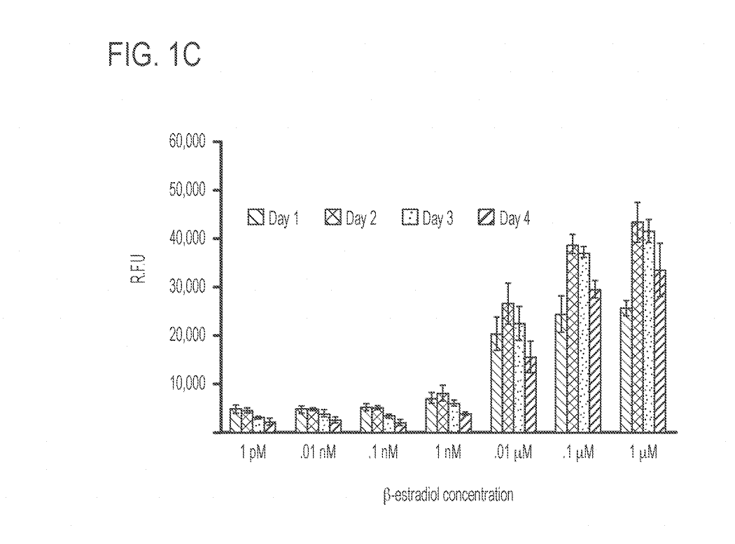

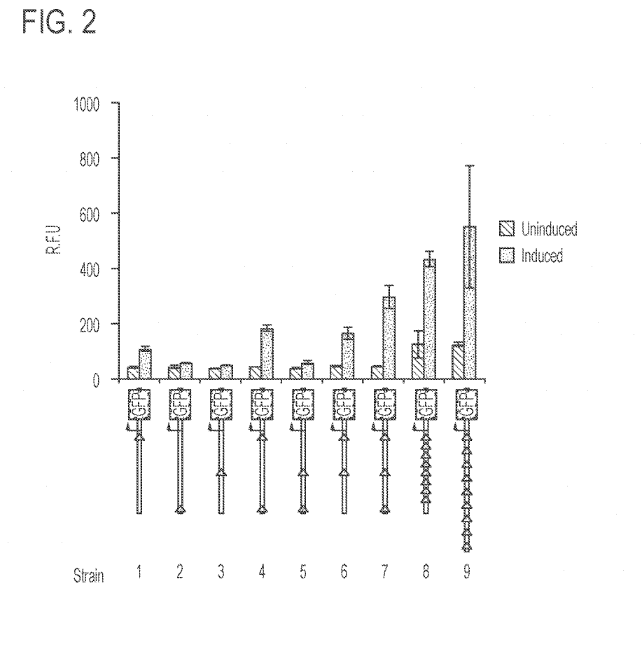

Gene expression was further optimized by modulating the number and placement of ZF-binding sites within the artificial promoter, which may be important parameters for maximizing the expression of regulated genes (Polstein et al. J. Am. Chem. Soc. (2012) 134, 16480-16483). A ppTEF1 promoter was used to express the ZF-TF, which targeted a minimal CYC promoter preceded by single ZF-binding sites placed ˜200, ˜350 or ˜500 base pairs upstream of the ATG start codon, as well as combinations of two or three ZF-binding sites (FIG. 2). A promoter with nine binding sites spaced ˜20 base pairs a part was tested (FIG. 2, Strain 8), and a promoter with nine ZF-binding sites spaced ˜40 base pairs a part was tested (with the closest ZF-binding site located 200 base pairs upstream of the ATG codon of the output gene) (FIG. 2, Strain 9). The results indicated that promoters with nine binding sites expressed GFP at higher levels than promoters with three binding sites when induced, whereas increasing the spacing between binding sites did not significantly improve levels of expression (FIG. 2).

Chromosomal context is an important factor to consider when expressing heterologous genes, because promoter interference, chromatin structure or other epigenetic modifications may have a negative impact on gene activation (Day et al. Genes Dev. (2000) 14, 2869-2880; Ramirez et al. Genetics (2001) 158, 341-350). Thus, GFP expression was assessed from cassettes integrated at nine different chromosomal loci. These loci correspond to regions targeted in prior reports (Cereghino et al. FEMS Microbiol. Rev. (2000) 24, 45-66; Chen et al. Micro. Cell Fact. (2012) 11, 91) or to intergenic regions, to avoid directly disrupting native coding sequences (Tables 1 and 2). No statistically significant differences in the maximally induced levels of gene expression were detected across nine loci tested (FIG. 8).

TABLE 1
DNA content of chromosomal DNA in K. phaffi

| Name | RefSeq | Size (Mb) | GC % | Genes |
|---|---|---|---|---|
| Chr 1 | NC_012963.1 | 2.8 | 41.0 | 1,538 |
| Chr 2 | NC_012964.1 | 2.39 | 41.0 | 1,333 |
| Chr 3 | NC_012965.1 | 2.25 | 41.1 | 1,198 |
| Chr 4 | NC_012966.1 | 1.78 | 41.4 | 971 |

TABLE 2
Chromosomal locations of homology regions used in the vectors that
were used to integrate landing pads in the genomic DNA of K. phaffi.

| Integration Site | Chromosome | Begin | End |
|---|---|---|---|
| 1 | 2 | 286,540 | 286,072 |
| 2 | 1 | 8,346 | 9,028 |
| 3 | 1 | 1,386,085 | 1,386,686 |
| 4 | 2 | 493,919 | 494,400 |
| 5 | 3 | 292,747 | 293,351 |
| 6 | 3 | 1,156,771 | 1,157,374 |
| 7 | 4 | 1,547,467 | 1,547,086 |
| 8 | 2 | 808,602 | 809,080 |
| 9 | 2 | 286,989 | 286,140 |

The performance of the ZF-inducible expression system can be further affected by additional parameters, such as the strength of the promoter driving expression of the ZF-TF and the basal activity of the minimal promoter, which contains ZF-binding sites, that controls expression of the output. The promoter driving ZF-TF expression is constitutive and determines how much ZF-TF accumulates in the cytoplasm. Excessive ZF-TF levels can potentially surpass the capacity of HSP90 to sequester the ZF-TF in the cytoplasm and, as a result, the ZF-TF may spontaneously translocate into the nucleus and activate expression in the absence of inducer, thus increasing undesired background. To minimize background expression levels, the minimal promoter containing ZF-binding sites should activate gene expression only when the inducer is present.

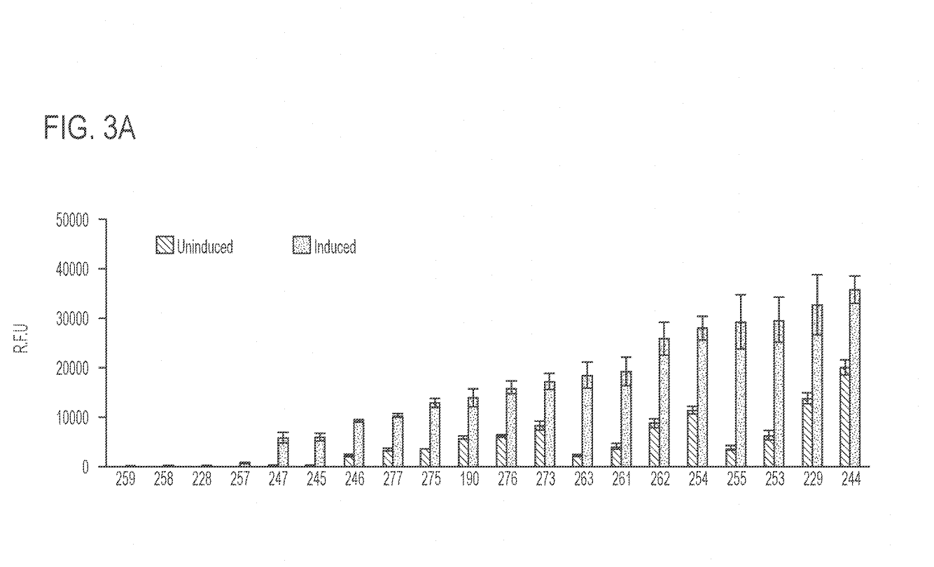

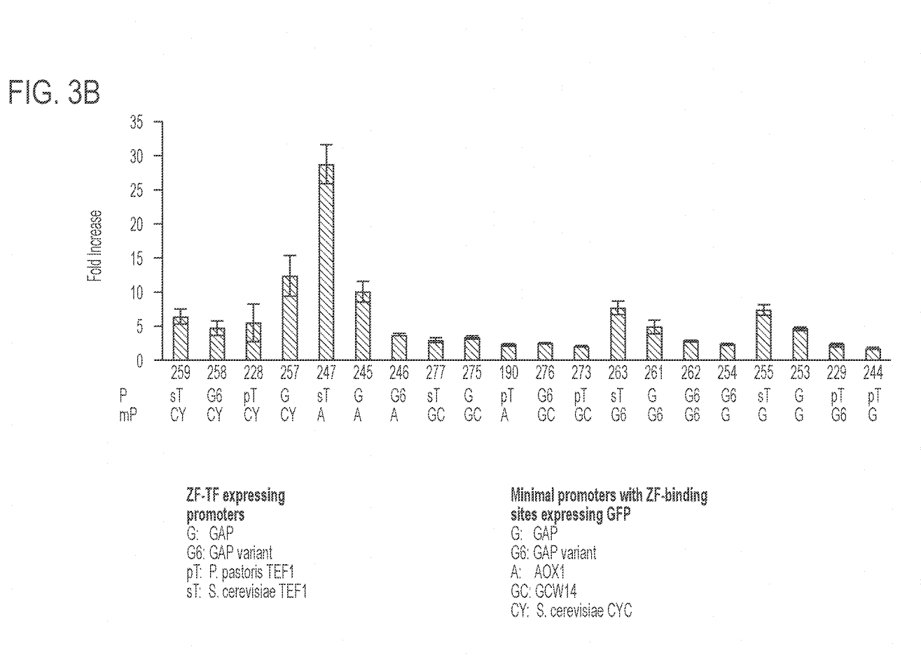

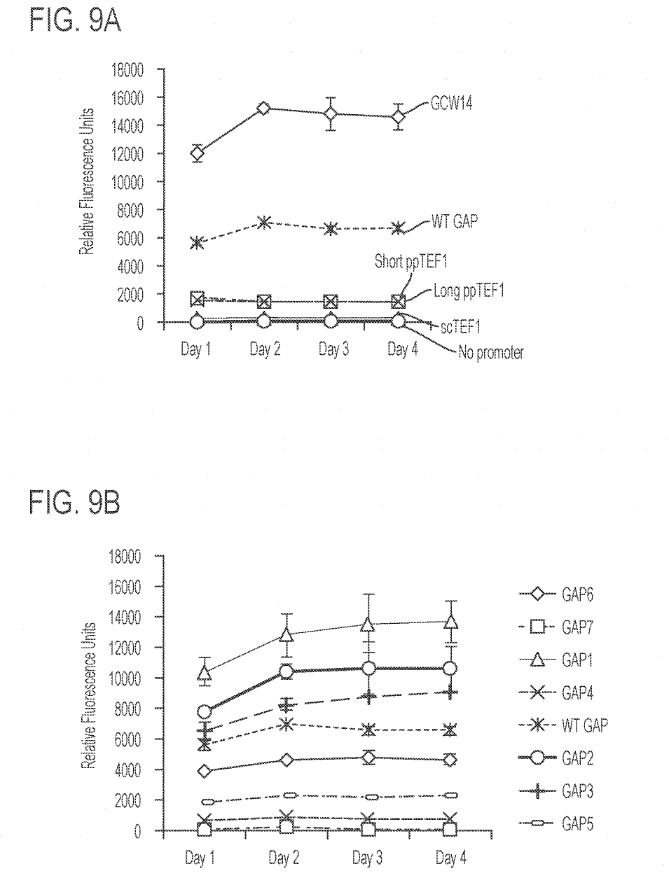

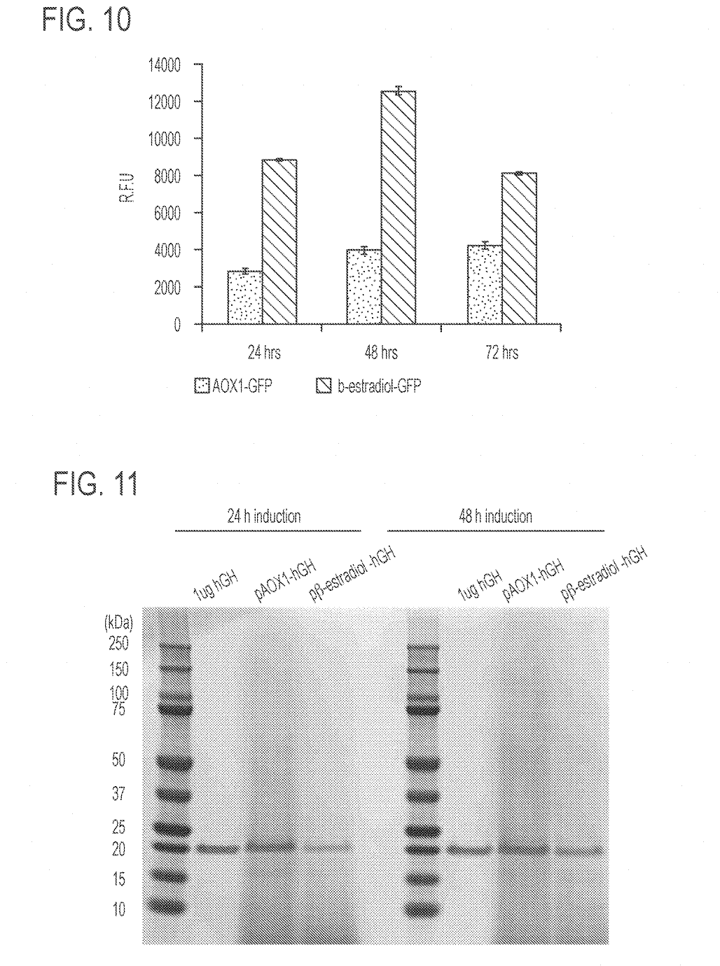

To fine-tune the expression of our output signal, the ZF-TF was expressed from several constitutive promoters previously used in K. phaffi, as well as variations of the GAP promoter constructed by introducing ˜50 bp insertions throughout its DNA sequence to modify its activity (FIG. 9). Combinations of these ZF-TF-expressing constitutive promoters with minimal promoters derived from those that regulate AOX1, GAP, a GAP6 variant, GCW14 and S. cerevisiae CYC1 (Table 3, FIGS. 3A and 3B). GFP expression via flow cytometry with and without induction with β-estradiol. The different combinations exhibited a wide range of background expression levels and maximal activation ratios (FIGS. 3A and 3B). Interestingly, some of the combinations consistently supported maximum expression levels higher than those reached by GFP expressed directly from the well-characterized PAOX1 under maximal induction (FIG. 10). Overall, combinations that exhibited higher maximum expression levels also had greater background levels in the OFF state.

TABLE 3
promoter combinations used in the figures:

| Strain | First promoter | Second promoter |
|---|---|---|
| 261 | GAP | GAP6 |
| 262 | GAP6 | GAP6 |
| 258 | GAP6 | mCYC |
| 259 | scTEF1 | mCYC |
| 255 | scTEF1 | GAP |
| 257 | GAP | mCYC |
| 253 | GAP | GAP |
| 254 | GAP6 | GAP |
| 263 | scTEF1 | GAP6 |
| 190 | ppTEF1 | AOX1 |
| 276 | GAP6 | GCW14 |
| 247 | scTEF1 | AOX1 |
| 277 | scTEF1 | GCW14 |
| 228 | ppTEF1 | mCYC |
| 245 | GAP | AOX1 |
| 229 | ppTEF1 | GAP6 |
| 244 | ppTEF1 | GAP |
| 275 | GAP | GCW14 |
| 246 | GAP6 | AOX1 |
| 273 | ppTEF1 | GCW14 |

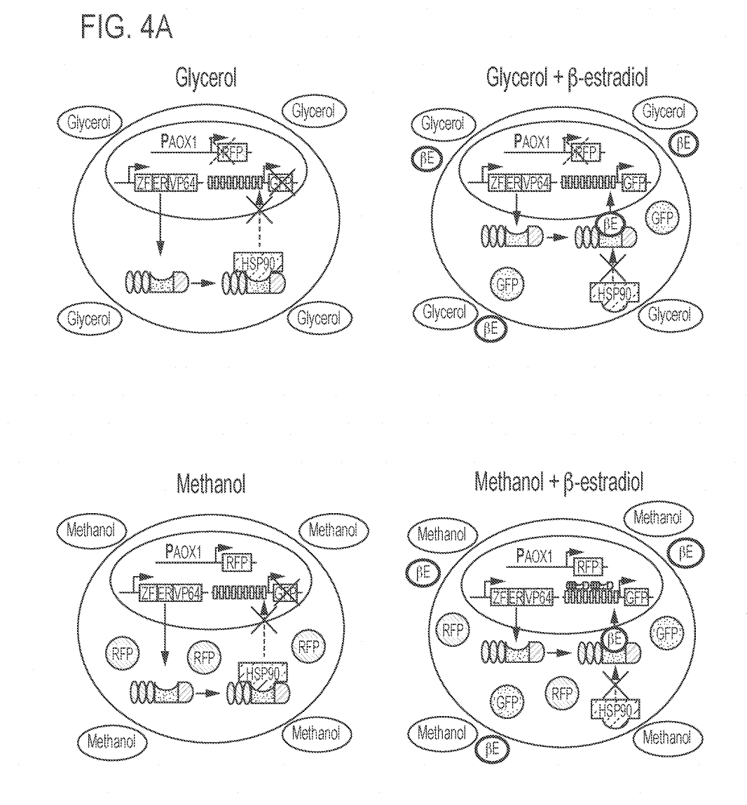

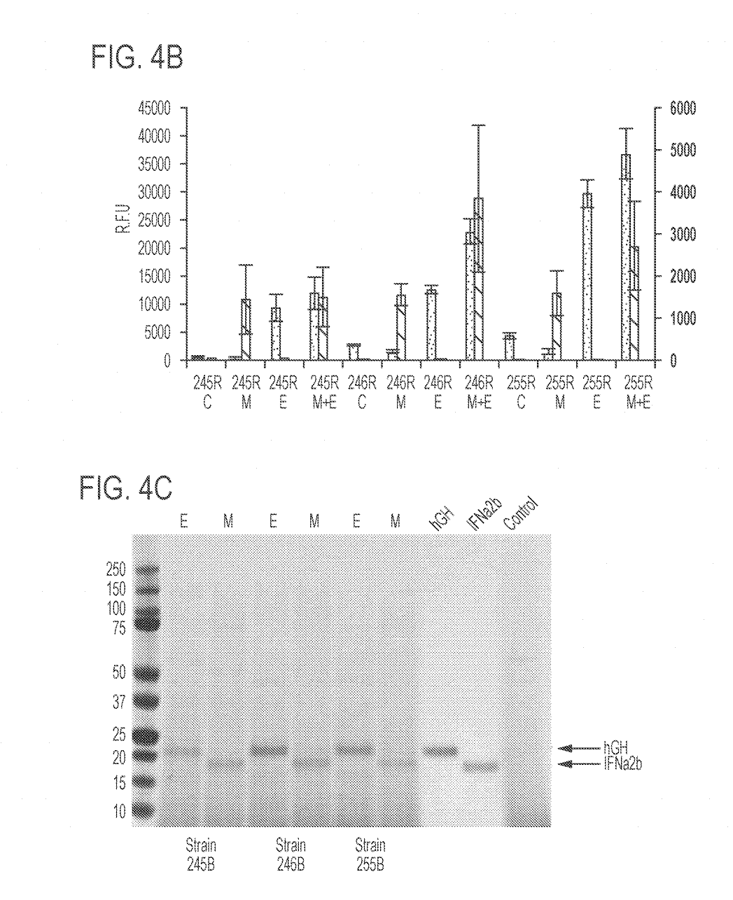

Three architectures were selected that had ON/OFF ratios in excess of ˜4-fold and/or high maximal expression activities for further engineering (architectures 245, 246 and 255; FIG. 3B). A second expression cassette was introduced to produce red fluorescent protein (RFP) using the AOX1 promoter to these vectors, resulting in the generation of strains 245R, 246R and 255R, respectively (FIG. 4A). As expected, these strains expressed GFP when induced with β-estradiol in buffered glycerol-complex medium (BMGY, “E”), expressed RFP when induced with buffered methanol-complex medium (BMMY, “M”), and expressed both GFP and RFP when induced with β-estradiol in BMMY (FIG. 4B, “M+E”).

The synthetic expression cassettes that were optimized with intracellular fluorescent reporters 245R, 246R and 255R (FIG. 4B), were used to develop proof-of-concept systems to controllably produce two biologic drugs, rHGH and IFNα2b. This resulted in the generation of three strains capable of selectable expression of two different polypeptides: strain 245B (GAP promoter expressing ZF-TF, AOX1 minimal promoter with ZF-binding sites expressing rHGH and AOX1 promoter expressing IFNα2b), strain 246B (GAP6 promoter expressing ZF-TF, AOX1 minimal promoter with ZF-binding sites expressing rHGH and AOX1 promoter expressing IFNα2b), and strain 255B (scTEF1 promoter expressing ZF-TF, GAP minimal promoter with ZF-binding sites expressing rHGH and AOX1 promoter expressing IFNα2b). Strains 245B, 246B and 255B were capable of selectable production of high-dose rHGH or IFNα2b in shake flasks within 24 hours of induction (FIG. 4C).

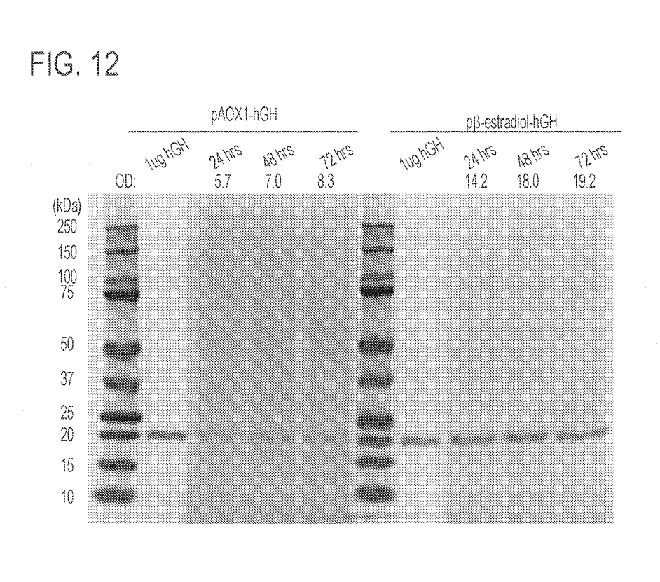

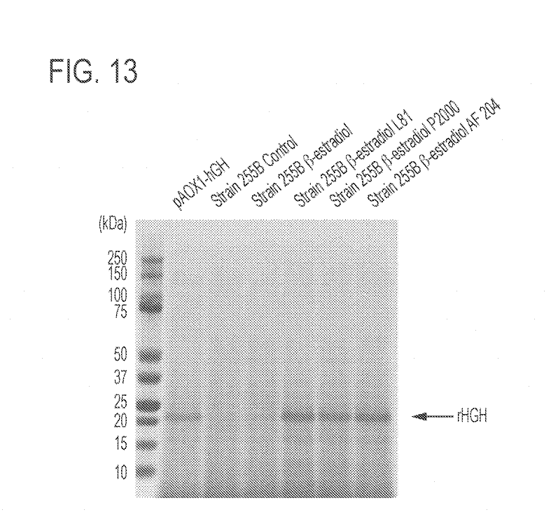

Interestingly, the level of protein (rHGH) secretion was similar when expressed from the AOX1 promoter or the β-estradiol-inducible system when expression was induced after 48 hours of outgrowth (FIG. 11). One difference between the systems is that β-estradiol can be used to induce protein secretion without outgrowth, while using glycerol as a carbon source, thereby allowing growth and production to occur simultaneously. Methanol induction, however, requires biomass accumulation (typically with glycerol) before the induction phase (Cereghino et al. FEMS Microbiol. Rev. (2000) 24, 45-66). Without prior biomass accumulation, protein production from AOX1 was significantly lower than production from a b-estradiol-inducible promoter (FIG. 12). As the daily dose of rHGH needed to treat patients is higher than the dose of IFNα2b, K. phaffi strains were engineered to express rHGH on β-estradiol induction and expression of IFNα2b on methanol induction. To maximize protein production, different media additives were also tested and determined that formulations containing the antifoams L81, P2000 and AF204 enhanced rHGH secretion levels from strain 255B, where GFP was replaced with rHGH (FIG. 13).

### Microbioreactor Production of Polypeptides

Sequential manufacturing of multiple biological therapeutics from individual strains requires rapid changes to their environment in high-density cell cultures. Furthermore, manufacturing at the volume scale of individual doses (for example, millilitres) suits requirements for point-of-care applications. For example, a production yield of 120 mg ml/L for IFNα2b in yeast (Degelmann et al. FEMS Yeast Res. (2002) 2, 349-361) could result in multiple doses when produced at the millilitre scale, as a common formulation of IFNα2b (Intron-A, db00105) is 11.6 mg (Law et al. Nucleic Acids Res (2014) 42, D1091-D1097). The large surface-area-to-volume ratio afforded by miniature systems facilitates rapid media changeover. To demonstrate this, a protocol was developed to be specifically tailored for programmable protein production with our engineered strains in an integrated, millilitre-scale table-top microbioreactor that can be operated continuously for point-of-care use in personal biomanufacturing, even with limited resources. By combining our engineered dual-biologics strain with the operational flexibility of the microbioreactor device, the traditional biomanufacturing approach was extended, where a single biologics is produced per process, into one that enables sequential or controllable expression of multiple different biologics.

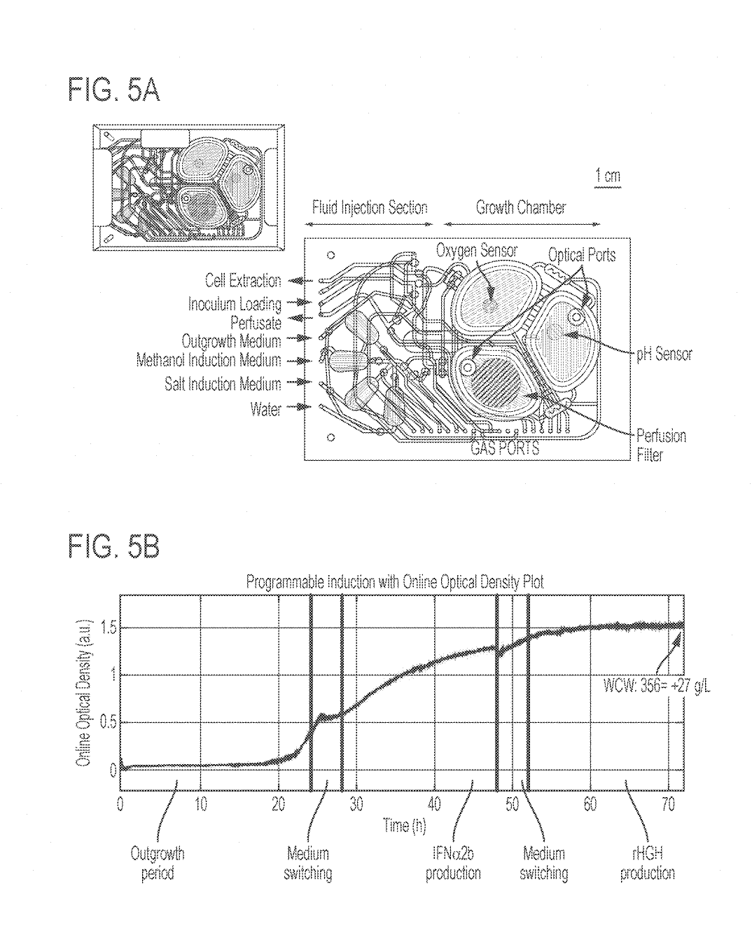

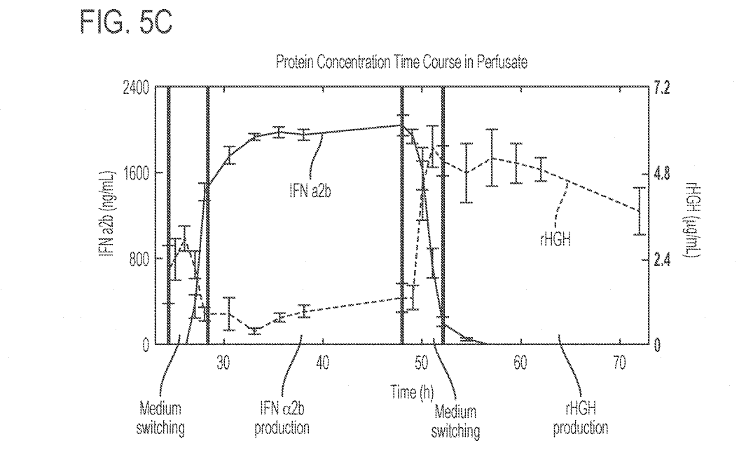

The principal component of the microbioreactor is a polycarbonate-PDMS membrane-polycarbonate sandwiched chip with active microfluidic circuits outfitted pneumatically for routing of reagents, precise peristaltic injection, growth chamber mixing and fluid extraction (FIG. 5A) (Lee et al. Lab Chip (2011) 11, 1720-1739). An injection volume of 700˜900 nl per injection was used for precise control of fluid addition/extraction in the 1-ml volume growth chamber. A 0.8-mm pore size perfusion filter (polyethersulfone) with a 1-cm diameter was incorporated underneath the growth chamber, to allow for fluid flow-through while maintaining all of the cells inside the growth chamber and enabling the switching of induction media (FIG. 5A). The ratio of the filter surface area to bioreactor volume was 0.758—a factor of 3 higher than high-performance, bench-scale perfusion bioreactors previously reported (Clincke et al. Biotechnol. Prog. (2013) 29, 754-767).

To demonstrate the paradigm of personalized, single-dose and programmable biomanufacturing, 3-day continuous cultivation experiments were performed for selectable production of two biologics at near-single-dose levels in <24 hours. The dual-polypeptide-producing K. phaffi strain 255B was inoculated from a single colony and grown in BMGY, first in batch and then in perfusion mode, with a perfusion rate of 0.5 ml/h. At 24 h, the outgrowth media was switched to the custom methanol media with perfusion rates of 1 ml/h for 4 hours for rapid changeover of the chemical environment. IFNα2b was collected at a perfusion rate of 0.5 ml/h for 20 hours. The 1 ml/h changeover rate was chosen to ensure that >98% of the preceding medium had been flushed out after this 4-hour medium changeover period according to the rate equation model for microbioreactor operation (see ‘Microbioreactor flow modelling’ in the Materials and Methods section). After the 48-hour time point, the custom methanol media was switched with the custom β-estradiol-containing media at a perfusion rate of 1 ml/h for 4 hours, followed by a collection phase for rHGH lasting 20 hours at a perfusion rate of 0.5 ml/h. A summary of the microbioreactor control operation is provided in Table 4. The different operational phases are illustrated with the online optical density (OD) plot in FIG. 5B. This experimental procedure resulted in 10 ml of perfusate for each protein production period lasting 20 hours each.

TABLE 4
Microbioreactor operation

| | | Flow | |
|---|---|---|---|
| Time | Medium | Rate | Purpose |
| 0 h | BMGY | 0 | Outgrowth |
| 14-24 h | BMGY | 0.5 ml/h | Outgrowth |
| 24-28 h | Methanol medium | 1.0 ml/h | Rapid medium switchover |
| 28-48 h | Methanol medium | 0.5 ml/h | IFNα2b production |
| 48-52 h | β-Estradiol medium | 1.0 ml/h | Rapid medium switchover |
| 52-72 h | β-Estradiol medium | 0.5 ml/h | rHGH production |

BMGY, buffered glycerol-complex medium;
IFNα2b, interferon-α2b;
rHGH, recombinant human growth hormone.
Summary table including the microbioreactor operation timeline, inflow medium, glow rate and purpose for different operation phases in FIG. 6b

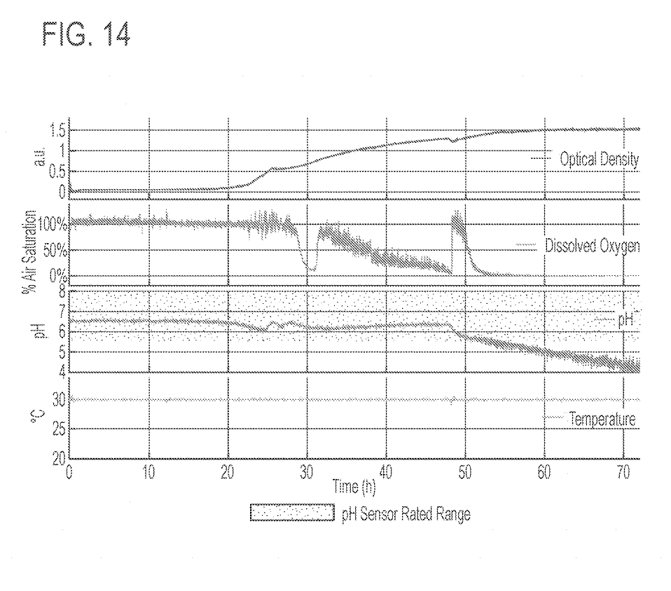

During the cultivation, cells inside the growth chamber were rapidly circulated and mixed by peristalsis. The fermentation temperature was controlled at 30±0.1° C. The online OD was recorded through an optical path length of ˜250 μm by a 630-nm light-emitting diode, where the optical path length is chosen to maximize the linear response range compatible with the fabrication process. The dissolved oxygen level of the culture was monitored online and controlled by dynamically changing the gas feed line between air and oxygen to match the dissolved oxygen set point, which was set to 100% air saturation in the experiment. The online pH data were also recorded during the fermentation process. The real-time sensor data for the microbioreactor experiments presented here can be found in FIG. 14. Four different input ports were used for the injection of BMGY outgrowth media, custom-made methanol media for IFNα2b production, β-estradiol-containing media for rHGH production and water for evaporation compensation. Perfusate was collected through the fluid channel downstream of the perfusion filter for protein characterization. For portable storage operation where lyophilized material may be used, the lyophilized material can be re-suspended in the reconstitution media before inoculation. This can serve as the seed inoculum and be injected into the growth chamber during the inoculation process. The cells could be revived inside the growth chamber subsequently without the need of additional steps. If the reconstitution media differs from the outgrowth media, a perfusion media changeover to the outgrowth media can be performed after the revival process.

To understand induction dynamics and protein secretion levels, samples were collected every hour during the 4-hour media switching periods. In addition, during the 20-hour production periods, four samples were collected every 2.5 hours and then one sample was collected after the last 10 hours. A standard enzyme-linked immunosorbent assay (ELISA) was carried out to quantify protein production. As shown in FIG. 5C, production profiles for the two biologics corresponded to the different induction media toggled by the microbioreactor. Four parallel microbioreactor experiments were carried out at the same time with identical protocols. Induction with methanol resulted in the rapid secretion of IFNα2b, which reached maximal productivity after only 3 hours and remained constant during the production period, and then rapidly decreased after methanol was removed from the media. Similarly, rHGH secretion was induced via media changeover to the custom β-estradiol-containing media, thus demonstrating multi-product expression control in the integrated platform.

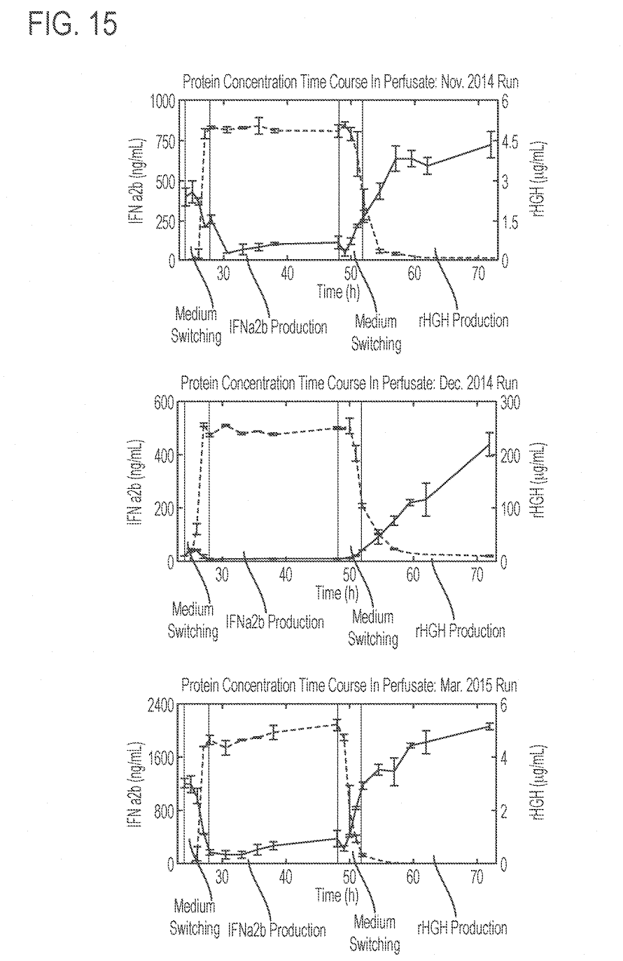

A summary of cumulative protein production and measurements of wet cell weight at the end of the experiment averaged across the four microbioreactors is shown in Table 5. The total average production of IFNα2b was 19.73 μg per reactor, which exceeds the 11.6 μg dose in Intron-A, whereas the total average production of rHGH was 43.7 μg per reactor (a common starting weight-based formulation of rHGH (Nutropin) is 0.006 mg kg/L per day (Drugs.com. www.drugs.com/dosage/nutropin-aq.html (2015)). Therefore, even without extensive bioprocess optimization, this system is capable of producing IFNα2b in excess of the daily dose needed in adults and matching the daily dose rHGH required to treat infants. Importantly, the cultivation conditions in the perfusion microbioreactor provided continuous nutrient supplies and high oxygen transfer rates (Lee et al. Lab Chip (2011) 11, 1730-1739) that led to the highest reported cell culture density achieved in any microfluidic platform, measured as an average wet cell weight of 356±27 g/L (Bareither et al. Biotechnol. Frog. (2011) 27, 2-14). In addition to the microbioreactor run shown in FIGS. 5B and 5C, three additional microbioreactor runs operating the same protocol were carried out and presented in FIG. 15.

TABLE 5
Microbioreactor production

| IFNα2b | rHGH | IFNα2b | rHGH | |
|---|---|---|---|---|
| Production (20 h) | Production (20 h) | Leakage (20 h) | Leakage (20 h) | Wet cell weight |
| 19.73 ± 0.72 μg | 43.7 ± 6.3 μg | 0.177 ± 0.032 μg | 10.8 ± 2.9 μg | 356 ± 27 g l⁻¹ |

IFNα2b, interferon-α2b;
rHGH, recombinant human growth hormone.
Production summary for IFNα2b and rHGH, and wet cell weight measurement at the end of the experiment. All data are averaged across four independent microbioreactors running the same protocol in parallel. Values represent mean and s.e.m. (n = 4)

### Discussion

The expression systems described herein can achieve ˜110-fold and ˜4-fold ON:OFF ratios for IFNα2b and rHGH, respectively. Using recombinase-based switches to invert protein-expressing DNA cassettes (Siuti et al. Nat. Biotechnol. (2013) 31, 448-452; Yang et al. Nat. Methods (2014) 11, 1261-1266) or additional repressors may further reduce leakage beyond the transcriptional systems used herein. For example, biologics-expressing cassettes could be surrounded by recombinase-recognition sites and initially encoded in inactive positions; expression of a recombinase could invert a targeted cassette and allow an upstream promoter to transcribe the correct messenger RNA. In addition, translational repressors or RNA interference could be used to further knock down undesirable expression levels in uninduced conditions. Combinatorial assembly of large numbers of genetic circuits followed by high-throughput screening for ones with enhanced ON:OFF ratios may further lower background expression. The integration of purification platforms with our biomanufacturing system may also reduce background levels of polypeptides in uninduced states. To circumvent the limited number of inducible systems available for controlling biologics expression, future work could integrate more advanced gene circuits, such as multiplexers that enable a restricted set of n inputs to control the expression of 2n outputs. Additional inducible systems that leverage orthogonal chemical inputs or non-chemical inputs, such as light, may increase the scalability of this system and, in the latter case, reduce logistics requirements.

The expression systems described herein may also be used for producing multi-component products, such as vaccines, by expressing the multiple products from a single strain. Furthermore, vaccines may be tailored for specific populations, as different antigens are likely to be optimal in providing immunity depending on geographical location or timing. With artificial regulation over the expression of different antigens, one could control the ultimate formulation of multi-component vaccines on demand for optimal prophylaxis and mitigate concerns about background expression. These vaccines could be customized to specific outbreaks or local conditions to enhance their applicability. Currently, the production of such multi-component vaccines can require multiple manufacturing lines, each with its own FDA approval. A multiplexed expression platform, such as those described herein, may be used for multi-component vaccines thereby reducing the regulatory burden for such products.

### Materials and Methods

Different media formulations namely BMGY, yeast extract peptone dextrose (YPD), BMMY, custom-made methanol medium and b-estradiol-containing salt medium were used in these experiments. BMGY medium contained 10 g/l (1% (w/v)) yeast extract (VWR catalogue #90004-092), 20 g/l (2% (w/v)) peptone (VWR catalogue #90000-264), 100 mM potassium phosphate monobasic (VWR catalog #MK710012), 100 mM potassium phosphate dibasic (VWR catalog #97061-588), 4×10⁻⁵% biotin (Life Technologies #B1595), 13.4 g/l (1.34% (w/v)) Yeast Nitrogen Base (Sunrise Science catalogue #1501-500) and 2% glycerol (VWR catalogue #AA36646-K7). YPD contained 1% yeast extract, 2% peptone and 2% dextrose (VWR catalogue # BDH0230). BMMY contained 1% yeast extract, 2% peptone, 100 mM potassium phosphate monobasic, 100 mM potassium phosphate dibasic, 4×10⁻⁵% biotin and 10 ml/1 (1% (v/v)) methanol (VWR catalogue #VWRCBDH20864.4). The custom-made methanol medium contained 1.34% Yeast Nitrogen Base, 0.79 g/l casaminoacids (Fisher #BP1424-500), 2% methanol (VWR catalogue #VWRCBDH20864.4) and 0.1% antifoam 204 (Sigma-Aldrich catalogue #A8311-50ML). The β-estradiol-containing salt medium contained 30 μM β-estradiol (Sigma-Aldrich catalogue #E4389-100MG), 18.2 g/l K2504 (VWR catalogue #97062-578), 7.28 g/l MgSO₄ (Sigma-Aldrich catalogue #M7506-500G), 4.3 g/l KOH (Fisher #P250-1), 0.08 g/l CaSO4 2H20 (Sigma Aldrich catalogue #C3771-500G), 13 ml/l 85% orthophosphoric acid (VWR catalogue #E582-50ML), 1.47 g/1 sodium citrate (Fisher #S279-500), 0.1% antifoam 204 and pH adjusted to 5.5 with ammonium hydroxide (Sigma-Aldrich catalogue #318612-500ML). All individual reagents were prepared as stock solutions and mixed immediately before the experiments. BMGY, custom-made methanol medium and b-estradiol-containing salt medium were used in the experiment for initial outgrowth, IFNα2b production and rHGH production, respectively.

### Plasmid Construction

The multiple constructs used in these experiments were built using conventional restriction enzyme cloning and/or Gibson assembly using the vector pPICZ A (Invitrogen #V190-20) as the backbone. All plasmids used in herein are described in Table 6 and have been deposited in the Addgene plasmid repository.

TABLE 6
List of constructs used herein

| Plasmid | | |
|---|---|---|
| Name | Notes | Addgene # |
| PP255 | P: scTEF1, mP: GAP | 78934 |
| PP43 | BxBI expression | 78953 |
| PP44 | R4 expression | 78954 |
| PP45 | TP901-1 expression | 78955 |
| PP295 | Strain 1 | 78935 |
| PP296 | Strain 2 | 78936 |
| PP297 | Strain 3 | 78937 |
| PP298 | Strain 4 | 78938 |
| PP299 | Strain 6 | 78939 |
| PP300 | Strain 7 | 78940 |
| PP318 | Strain 5 | 78941 |
| PP228 | Strain 8 | 78942 |
| PP322 | Strain 9 | 78943 |
| PP259 | P: scTEF1, mP: mCYC | 78961 |
| PP258 | P: GAP6, mP: mCYC | 78962 |
| PP228 | P: ppTEF1, mP: mCYC | 78942 |
| PP257 | P: GAP, mP: mCYC | 78963 |
| PP247 | P: scTEF1, mP: AOX1 | 78964 |
| PP245 | P: GAP, mP: AOX1 | 78965 |
| PP246 | P: GAP6, mP: AOX1 | 78966 |
| PP277 | P: scTEF1, mP: GCW14 | 78967 |
| PP275 | P: GAP, mP: GCW14 | 78968 |
| PP190 | P: ppTEF1, mP: AOX1 | 78969 |
| PP276 | P: GAP6, mP: GCW14 | 78970 |
| PP273 | P: ppTEF1, mP: GCW14 | 78971 |
| PP263 | P: scTEF1, mP: GAP6 | 78972 |
| PP261 | P: GAP, mP: GAP6 | 78973 |
| PP262 | P: GAP6, mP: GAP6 | 78974 |
| PP254 | P: GAP6, mP: GAP | 78975 |
| PP255 | P: scTEF1, mP: GAP | 78934 |
| PP253 | P: GAP, mP: GAP | 78976 |
| PP229 | P: ppTEF1, mP: GAP6 | 78977 |
| PP244 | P: ppTEF1, mP: GAP | 78978 |
| PP310 | 245R P: GAP, mP: AOX1 | 78979 |
| PP311 | 246R P: GAP6, mP: AOX1 | 78980 |
| PP362 | 255R P: scTEF1, mP: GAP | 78981 |
| PP324 | 246B P: GAP6, mP: AOX1 | 78982 |
| PP326 | 245B P: GAP, mP: AOX1 | 78983 |
| PP363 | 255B P: scTEF1, mP: GAP | 78984 |
| PP74 | Integration Site 1 | 78944 |
| PP75 | Integration Site 2 | 78945 |
| PP76 | Integration Site 3 | 78946 |
| PP77 | Integration Site 4 | 78947 |
| PP78 | Integration Site 5 | 78948 |
| PP79 | Integration Site 6 | 78949 |
| PP69 | Integration Site 7 | 78950 |
| PP67 | Integration Site 8 | 78951 |
| PP151 | Integration Site 9 | 78952 |
| PP165 | GCW14 | 78985 |
| PP152 | Long ppTEF1 | 78986 |
| PP87 | scTEF1 | 78987 |
| PP164 | Short ppTEF1 | 78988 |
| PP149 | WT GAP | 78989 |
| PP093 | No promoter | 78990 |
| PP154 | GAP1 | 78991 |
| PP160 | GAP2 | 78992 |
| PP161 | GAP3 | 78993 |
| PP155 | GAP4 | 78994 |
| PP162 | GAP5 | 78995 |
| PP163 | GAP6 | 78996 |
| PP153 | GAP7 | 78997 |

### Strains

The wild-type K. phaffi (P. pastoris) strain NRRL Y-11430, ATCC 76273, was used in the experiments described herein.

### Electroporation

Competent cells were prepared by first growing one single colony of K. phaffi (P. pastoris) in 5 ml YPD at 30° C. overnight. Fifty microlitres of the resulting culture were inoculated 100 ml of YPD and grown at 30° C. overnight again to an OD600 B1.3-1.5. The cells were the centrifuged at 1,500 g for 5 min at 4° C. and resuspended with 40 ml of ice-cold sterile water, centrifuged at 1,500 g for 5 min at 4° C. and resuspended with 20 ml of ice-cold sterile water, centrifuged at 1,500 g for 5 min at 4° C. and resuspended in 20 ml of ice-cold 1M sorbitol, and centrifuged at 1,500 g for 5 min at 4° C. and resuspended in 0.5 ml of ice-cold 1M sorbitol. For transformation of the landing pad plasmids, 80 ml of competent cells were mixed with 5-20 mg of linearized DNA and transferred to an ice-cold 0.2 cm electroporation cuvette for 5 min. For recombinase-based transformations, 10 mg of circular transfer vector and 10 mg of circular recombinase expression vector were combined, added to 80 ml of competent cells and incubated for 5 min in an ice-cold 0.2 cm electroporation cuvette. Pulse parameters were 1,500V, 200Ω and 25 μF Immediately after pulsing, 1 ml of ice-cold 1M sorbitol was added to the cuvette and the cuvette content was transferred to a sterile culture tube containing 2X YPD. The culture tubes were incubated for 2 h at 30° C. with shaking and 50-100 ml of the culture was spread on plates (1% yeast extract, 2% peptone, 1M sorbitol, 1% dextrose and 2% agar) with the appropriate selection antibiotic (zeocin 100 mg/mL G418 100 mg/mL).

### Cell Induction and Flow Cytometry

One single colony was grown in 1 ml of BMGY in a 12-ml culture tube at 30° C. in a shaking incubator (250-300 r.p.m.) overnight. The cells were centrifuged at 500 g for 5 min at room temperature and washed twice with PBS. After the second wash, the cells were resuspended in induction medium consisting of BMMY or BMGY with β-estradiol. After 24 h, the cultures were centrifuged at 500 g for 5 min at room temperature, washed with 1 ml of PBS twice, resuspended in 1 ml of PBS and used for flow cytometry with a BD LSR Fortessa™ cell analyser.

### ELISA Assay

The concentration of hGH and IFN-α2b in each of the samples was determined by ELISA assay. Solid-phase 96-well ELISA plates were used, specifically designed for the quantification of hGH (Quantikine ELISA, R&D Systems) and IFN-α (Verikine Human IFN-alpha ELISA Kit, PBL Assay Science). The products were provided with the proper standard stock solutions or powder for each of the assays. For hGH, the standard curve was calculated using concentrations ranging between 3,200 and 25 pg/l, in twofold serial dilutions. For IFNα2b, the extended range standard curve was used, with an additional large concentration point: the standard concentration varied between 156 and 10,000 pg/l, in twofold serial dilutions. For both assays, negative controls were also included. Before the protein quantification, the samples were diluted in the appropriate buffer so that the concentrations determined would fall within the assay and equipment limits.

The optical density values obtained for each of the assays were plotted using a four-parameter fit for the standard curve. Each sample was measured twice and the results represent the average and standard deviation of three biological replicates.

### Quantitative PCR

Genomic DNA from different strains was isolated using the YeaStar Genomic DNA kit (Zymo) and the resulting preps were diluted down to a concentration of 5 ng/uL. qPCR mixtures were prepared using the LightCycler 480 SYBR Green Master mix (Roche) with 10 ng genomic DNA from each strain and 400 nM of each primer per assay in a total reaction volume of 20 ml. Reactions were performed in LightCycler 480 96-well reaction plates in triplicate with a standard curve for each gene generated through tenfold dilutions from 2 ng of plasmid containing the gene-of-interest. The amplification conditions were as follows: 95° C. for 10 min followed by 45 cycles of 95° C. for 10 s, 56° C. for 15 s and 72° C. for 20 s. The amplification period was followed by a melting curve analysis with a temperature gradient of 0.1 !C s!1 from 65° C. to 95° C. An amplicon from Q6 the single-copy GAPDH or ACT1 genes was used for normalization. Using the published genome size of 9.4 Mbp, we expected 98,000 copies of the genome to be present in 1 ng haploid K. phaffi (P. pastoris) genomic DNA. Absolute copy number for the gene-of-interest in each strain was calculated using the mean Ct value and the corresponding standard curve.

The sequence of the primers used were as follows:

hGH
(5′-GGGCAGATCTTCAAGCAGAC-3′ (SEQ ID NO: 16),
5′-CTCGACCTTGTCCATGTCCT-3′ (SEQ ID NO: 17));
IFN
(5′-TTCCCACAAGAGGAATTTGG-3′ (SEQ ID NO: 18),
5′ AGGCTGCTGAGGAATCTTTG-3′ (SEQ ID NO: 19));
GAPDH
(5′-TGGGTTACACTGAAGATGCC-3′ (SEQ ID NO: 20),
5′-CGTTGTCGTACCAAGAGATCAG-3′ (SEQ ID NO: 21));
ACT1
(5′-TGGTATCGTTTTGGACTCTGG-3′ (SEQ ID NO: 22),
5′-AGCGTGTGGTAAGGAGAAAC-3′ (SEQ ID NO: 23));
and
landing pad
(5′-TGTCTTCGTGGTTTGTCTGG-3′ (SEQ ID NO: 24),
5′-TCTTGTAGTTGCCGTGTCG-3′ (SEQ ID NO: 25)).

### Microbioreactor

Parts for the microbioreactor experiment were purchased from Pharyx Inc. A single bioreactor control hub (Pharyx, #MBS-004) with an overall footprint of 31 cm (w)#34 cm (d)#36 cm (h) was used to control four independent microbioreactor units (Pharyx, #MCM-001) for the fermentation experiment. Each microbioreactor unit interfaces with a single-use disposable microfluidic chip made of a sandwiched polycarbonate-PDMS membrane polycarbonate structure (Pharyx, #CCPST-1) to carry out peristaltic control and online sensing. The design, fabrication and system configuration for the microbioreactor have been described previously (Lee et al. Lab Chip (2011) 11, 1730-1739; Lee et al. Lab Chip (2009) 9, 1618-1624). To provide the rapid medium changeover required for this study, a perfusion filter (Pall Corp., Supor 800, #60109) was incorporated into the cultivation chamber (Mozdzierz et al. Lab Chip (2015) 15, 2918-2922) to allow medium flow-through, while maintaining cells inside the growth chamber.

### Microbioreactor Flow Modelling

To account for the microbioreactor media concentration dynamics during the changeover period, a simple model based on fluid concentration rate equation under equal inflow and outflow rate was constructed. For simplicity, cellular consumption on media material was not taken into account in this model. Assuming that S(t) represents a particular medium concentration in the growth chamber, Sᵢₙ (t) represents the concentration of this medium in the input flow, F represents the input flow rate and V represents the growth chamber volume, then the rate equation for the medium concentration in the growth chamber can be modelled as

$$\frac{\partial s ( t )}{\partial t} = \frac{F}{V} [ {S}_{in} ( t ) - S ( t ) ] .$$

For the situation of introducing a completely new medium into the growth chamber to replace an old medium similar to our programmable biologics production experiment, assuming Sᵢₙ (t) is constant during the time of changeover and t=0 being the start of changeover event, the new medium concentration can be simply solved analytically as

$${S}_{new} ( t ) = {S}_{in} ( 1 - {e}^{ ⋀} ( \frac{- F t}{V} ) ) .$$

Meanwhile, the concentration of the old medium due to flush out follows as

$${S}_{old} ( t ) = {S}_{old} {e}^{⋀} ( \frac{- F t}{V} ) ,$$

assuming S_{old} is the concentration of the old medium before the changeover event. Therefore, a flow rate of 1 ml/h for the changeover period of 4 hours would result in a medium replacement percentage of ˜1-e⁻⁴≈98.2%. In contrast, if the changeover rate stays as 0.5 ml/h similar to the case for the production period, the medium replacement percentage would be ˜1-e⁻²≈86.5%, where there would still be a substantial amount of the previous medium leftover after the changeover event. Such physical modelling of the microbioreactor operation provides a simple yet effective design guideline to complement the operational flexibility of our manufacturing platform.

### Microbioreactor Experiment

The microbioreactor chips were γ-irradiated and sealed as part of the standard pre-inoculation sterile protocol. The medium bottles and feed lines were autoclaved separately. The initial inoculum was loaded from a single colony from a YPD plate stored at 4° C. at 0 h. The fermentation parameter plot for the online OD, dissolved oxygen, pH and temperature for one experiment is shown in FIG. 14. As described in the manuscript, the fermentation temperature was controlled at 30±0.1° C. throughout the entire experiment. The dissolved oxygen level of the microbioreactor was controlled by dynamically changing the gas feed line between air and oxygen to match the dissolved oxygen set point, which was set to 100% air saturation in the experiment. As the cells grow and the overall oxygen consumption rate increases, the gas controller gradually increases the oxygen content in the gas feed line to maintain the dissolved oxygen set point. Once the cell oxygen consumption rate overpasses the oxygen transfer rate by pure oxygen supply, the dissolved oxygen drops below the set point and the supply gas remains at 100% oxygen. The online OD is monitored through light scattering across an optical path length of ˜250 μm inside the growth chamber with a 630-nm light-emitting diode. The linear response range for this OD sensor is around 0˜0.7 online OD unit. Above ˜0.7 online OD unit, the sensor reading no longer increases linearly with the cell density. The online pH data are also recorded during the fermentation process. The pH sensor is rated for pH values of 5.5˜8.5. During the third day, the pH reading falls below 5.5 and therefore may not accurately represent the actual pH of the culture environment. Over the course of the study, perfusate samples were collected downstream of the perfusion filter and were stored at 4° C. before processing with ELISA assay.

### Additional Microbioreactor Runs

In addition to the microbioreactor run presented in the body manuscript, three additional runs of the microbioreactor experiments were performed. Each run is marked by the time of the experiment and consists of two independent microbioreactors operating the same protocol in parallel. The protein concentration time course plot is shown in FIG. 15 and the cumulative protein production quantity is summarized in Table 7. Overall, the same switching behavior is observed across all runs with some run-to-run protein production variations observed, especially for the run labeled “December 2014 Run” (FIG. 15, top right panel) where rHGH production was much higher than the others. These run-to-run variations may be caused by experimentation with the inner surface coating and γ-irradiation protocol of the microbioreactor chip. After the microbioreactor parts were fabricated and bonded, an inner surface coating protocol with PEG treatment was carried out, followed by a chip nitrogen purge and bagging procedure, and γ-irradiation sterilization. Some experimentation on this protocol in terms of PEG-silane treatment time and chip nitrogen purge procedure in search for the optimal coating condition was carried across the early microbioreactor runs. For all runs, the chips used in the same run were fabricated with exactly the same protocol and therefore do not cause much variation within each microbioreactor run. The chip fabrication protocol was optimized, resulting in a substantial reduction in run-to-run variation. Nonetheless, the results indicate the potential of high production yield comparable to industrial level for the manufacturing platform described herein with suitable manufacturing standardization and bioprocess optimization.

TABLE 7
Production summary for IFNα2b and rHGH and wet cell weight measurement for
the three additional microbioreactor runs in FIG. 15.

| | IFN α2b | rHGH | IFN α2b | rHGH | |
|---|---|---|---|---|---|
| | Production | Production | Leakage | Leakage | Wet Cell |
| | (20 hours) | (20 hours) | (20 hours) | (20 hours) | Weight |
| November 2014 | 8.17 ± 0.31 μg | 38.9 ± 4.2 μg | 0.223 ± 0.033 μg | 5.45 ± 1.66 μg | 387 ± 44 g/L |
| Run | | | | | |
| December 2014 | 4.93 ± 0.04 μg | 1.52 ± 0.18 mg | 0.319 ± 0.026 μg | 0.03 ± 0.0079 mg | 349 ± 8 g/L |
| Run | | | | | |
| March 2015 | 19.78 ± 0.74 μg | 45.8 ± 2.2 μg | 0.137 ± 0.026 μg | 7.09 ± 2.36 μg | 351 ± 26 g/L |
| Run | | | | | |

All data are averaged across two independent microbioreactors running the same protocol in parallel. Values represent mean and s.e.m. (n=2).

### REFERENCES FOR EXAMPLE 1

1. Thiel, K. A. Biomanufacturing, from bust to boom . . . to bubble? Nat. Biotechnol. 22, 1365-1372 (2004).
2. Farid, S. S., Thompson, B. & Davidson, A. in 10th Annual bioProcess UK Conference, MAbs 6, 1357-1361 (3-4, December 2013, London, U K, 2014).
3. Hwang, T. J. Stock market returns and clinical trial results of investigational compounds: an event study analysis of large biopharmaceutical companies. PLoS ONE 8, e71966 (2013).
4. Ledford, H. First biosimilar drug set to enter US market. Nature 517, 253-254 (2015).
5. Siganporia, C. C., Ghosh, S., Daszkowski, T., Papageorgiou, L. G. & Farid, S. S. Capacity planning for batch and perfusion bioprocesses across multiple biopharmaceutical facilities. Biotechnol. Prog. 30, 594-606 (2014).
6. Willke, R. J. et al. Melding regulatory, pharmaceutical industry, and U.S. payer perspectives on improving approaches to heterogeneity of treatment effect in research and practice. Value Health 16, S10-S15 (2013).
7. Zhu, J. Mammalian cell protein expression for biopharmaceutical production. Biotechnol. Adv. 30, 1158-1170 (2012).
8. Goldrick, S., Stefan, A., Lovett, D., Montague, G. & Lennox, B. The development of an industrial-scale fed-batch fermentation simulation. J. Biotechnol. 193, 70-82 (2015).
9. Diel, B., Manzke, C. & Peuker, T. Flexible biomanufacturing processes that address the needs of the future. Adv. Biochem. Eng. Biotechnol. 138, 207-237 (2014).
10. Berlec, A. & Strukelj, B. Current state and recent advances in biopharmaceutical production in Escherichia coli, yeasts and mammalian cells. J. Ind. Microbiol. Biotechnol. 40, 257-274 (2013).
11. Mattanovich, D. et al. Recombinant protein production in yeasts. Methods Mol. Biol. 824, 329-358 (2012).
12. Vervecken, W. et al. In vivo synthesis of mammalian-like, hybrid-type N-glycans in Pichia pastoris. Appl. Environ. Microbiol. 70, 2639-2646 (2004).
13. Vervecken, W., Callewaert, N., Kaigorodov, V., Geysens, S. & Contreras, R. Modification of the N-glycosylation pathway to produce homogeneous, human-like glycans using GlycoSwitch plasmids. Methods Mol. Biol. 389, 119-138 (2007).
14. Zhang, N. et al. Glycoengineered Pichia produced anti-HER2 is comparable to trastuzumab in preclinical study. MAbs 3, 289-298 (2011).
15. Macauley-Patrick, S., Fazenda, M. L., McNeil, B. & Harvey, L. M. Heterologous protein production using the Pichia pastoris expression system. Yeast 22, 249-270 (2005).
16. Vogl, T., Hartner, F. S. & Glieder, A. New opportunities by synthetic biology for biopharmaceutical production in Pichia pastoris. Curr. Opin. Biotechnol. 24, 1094-1101 (2013).
17. Prielhofer, R. et al. Induction without methanol: novel regulated promoters enable high-level expression in Pichia pastoris. Microb. Cell Fact. 12, 5 (2013).
18. Koller, A., Valesco, J. & Subramani, S. The CUP1 promoter of Saccharomyces cerevisiae is inducible by copper in Pichia pastoris. Yeast 16, 651-656 (2000).
19. Resina, D., Cos, O., Ferrer, P. & Valero, F. Developing high cell density fed-batch cultivation strategies for heterologous protein production in Pichia pastoris using the nitrogen source-regulated FLD1 promoter. Biotechnol. Bioeng. 91, 760-767 (2005).
20. Cereghino, J. L. & Cregg, J. M. Heterologous protein expression in the methylotrophic yeast Pichia pastoris. FEMS Microbiol. Rev. 24, 45-66 (2000).
21. Yamaguchi, S. et al. A method for producing transgenic cells using a multi-integrase system on a human artificial chromosome vector. PLoS ONE 6, e17267 (2011).
22. Khalil, A. S. et al. A synthetic biology framework for programming eukaryotic transcription functions. Cell 150, 647-658 (2012).
23. McIsaac, R. S. et al. Synthetic gene expression perturbation systems with rapid, tunable, single-gene specificity in yeast. Nucleic Acids Res. 41, e57 (2013).
24. Perez-Pinera, P. et al. Synergistic and tunable human gene activation by combinations of synthetic transcription factors. Nat. Methods 10, 239-242 (2013).
25. Polstein, L. R. & Gersbach, C. A. Light-inducible spatiotemporal control of gene activation by customizable zinc finger transcription factors. J. Am. Chem. Soc. 134, 16480-16483 (2012).
26. Day, C. D. et al. Transgene integration into the same chromosome location can produce alleles that express at a predictable level, or alleles that are differentially silenced. Genes Dev. 14, 2869-2880 (2000).
27. Ramirez, A. et al. Sequence and chromosomal context effects on variegated expression of keratin 5/lacZ constructs in stratified epithelia of transgenic mice. Genetics 158, 341-350 (2001).
28. Chen, M. T. et al. Generation of diploid Pichia pastoris strains by mating and their application for recombinant protein production. Microb. Cell Fact. 11, 91 (2012).
29. Degelmann, A. et al. Strain and process development for the production of human cytokines in Hansenula polymorpha. FEMS Yeast Res. 2, 349-361 (2002).
30. Law, V. et al. DrugBank 4.0: shedding new light on drug metabolism. Nucleic Acids Res. 42, D1091-D1097 (2014).
31. Lee, K. S., Boccazzi, P., Sinskey, A. J. & Ram, R. J. Microfluidic chemostat and turbidostat with flow rate, oxygen, and temperature control for dynamic continuous culture. Lab Chip 11, 1730-1739 (2011).
32. Clincke, M. F. et al. Very high density of CHO cells in perfusion by ATF or TFF in WAVE bioreactor. Part I. Effect of the cell density on the process. Biotechnol. Prog. 29, 754-767 (2013).
33. Drugs.com. http://www.drugs.com/dosage/nutropin-aq.html (2015).
34. Bareither, R. & Pollard, D. A review of advanced small-scale parallel bioreactor technology for accelerated process development: current state and future need. Biotechnol. Prog. 27, 2-14 (2011).
35. Siuti, P., Yazbek, J. & Lu, T. K. Synthetic circuits integrating logic and memory in living cells. Nat. Biotechnol. 31, 448-452 (2013).
36. Yang, L. et al. Permanent genetic memory with 41-byte capacity. Nat. Methods 11, 1261-1266 (2014).
37. Warikoo, V. et al. Integrated continuous production of recombinant therapeutic proteins. Biotechnol. Bioeng. 109, 3018-3029 (2012).
38. Lee, K. S. & Ram, R. J. Plastic-PDMS bonding for high pressure hydrolytically stable active microfluidics. Lab Chip 9, 1618-1624 (2009).
39. Mozdzierz, N. J. et al. A perfusion-capable microfluidic bioreactor for assessing microbial heterologous protein production. Lab Chip 15, 2918-2922 (2015).

### Example 2: Simultaneous, Versatile, and On-Demand Production of Multiple Therapeutic Biologics

On-demand drug manufacturing can be useful for research, clinical studies, or urgent therapeutic use, but is challenging when more than one drug at a time is needed or resources are scarce. Here, we propose multitasking strategies to produce multiple biologics concurrently in single batches from yeast by multiplexing strain development, cell culture, separation, and purification. We demonstrated three biologics co-production strategies: i) inducible expression of multiple biologics and control over the ratio between biologic drugs produced together; ii) a consolidated bioprocessing platform; and iii) co-expression and co-purification of a mixture of two monoclonal antibodies. We used these basic strategies to implement a more complex system to produce drug mixtures and demonstrated the separation of these drugs. Finally, we achieved scalable modulation of yeast biologics production via three orthogonal small-molecule inducible gene expression systems. These multitasking strategies offer a diverse array of options for flexible, on-demand, and decentralized biomanufacturing applications without the need for specialized equipment.

The shortage of essential drugs is of global concern^{1,2}, especially in developing countries. Transportation infrastructure is often inadequate, and the timely delivery of drugs to remote locations is difficult. Even in developed countries, emergency situations can compromise the supply of important medicines, such as the insulin shortage crisis in New Orleans after Hurricane Katrina³, or raise the risk of infectious disease outbreaks. On-site, small-scale drug manufacturing can provide drugs on demand for isolated or inaccessible regions⁴⁻⁶. However, it is difficult to precisely predict the types and amounts of drugs needed in a certain region and time, so a large number of strains have to be cultivated and multiple facilities built in order to generate a large supply of needed drugs. High capital investment and maintenance costs and low utilization rates make such production difficult in regions with limited resources. Therefore, it would be of great interest to have a versatile platform to manufacture a variety of different drugs on demand with low capital investment. Biologics manufacturing involves four phases: strain/cell line construction, upstream processing (fermentation), downstream purification, and drug formulation. Usually, each biologic is produced in one strain within a manufacturing facility. Although economically efficient for large-scale production in biopharmaceutical plants, this method is inefficient and time-consuming for small-scale production, which would be useful for single-dose production, lab-scale research, and clinical studies^{7,8}.

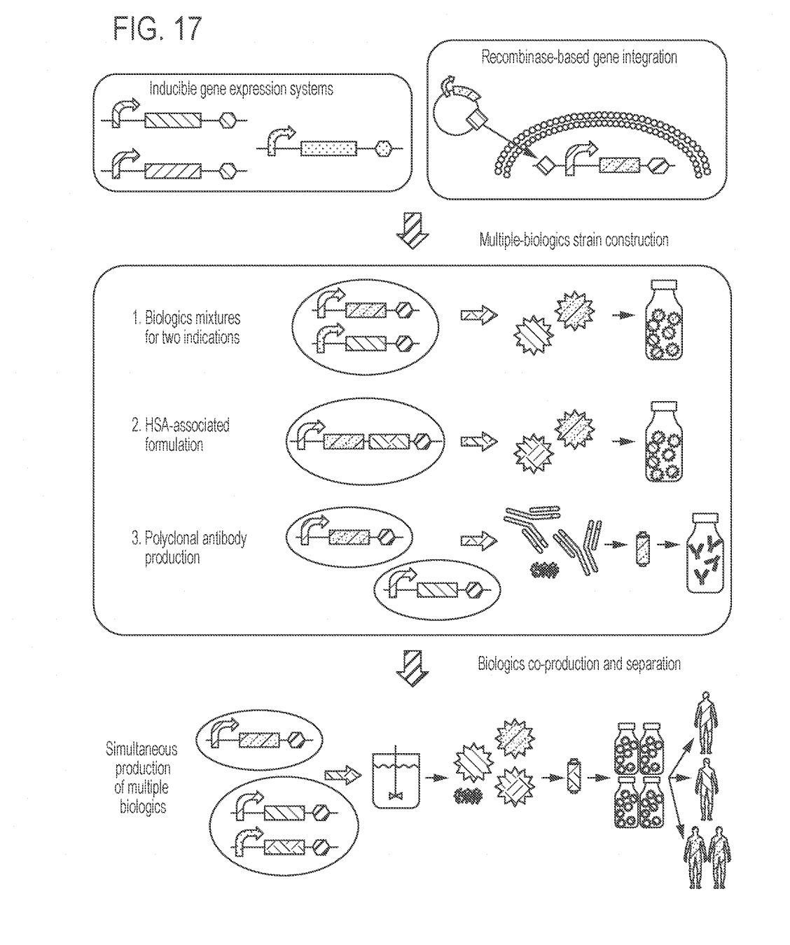

We envision that performing multiple bioprocesses simultaneously can overcome challenges in portable and/or small-scale biologics manufacturing. We propose to co-produce multiple drugs in a single batch via a versatile platform (FIG. 17) that: i) can generate several drugs on demand rather than one by one; ii) can enable control over the ratio of co-produced drugs and reduce the overall manufacturing time; iii) can separate and purify drugs in a two-stage downstream process to efficiently recover products and eliminate cross-contamination. This co-production strategy can also be used to manufacture combination drugs. Combination drugs contain two or more active pharmaceutical ingredients (APIs) and can have synergistic effects on a single disease or confer broad protection or treatments⁹. For example, cocktails consisting of multiple antiretroviral drugs are widely used against HIV¹⁰, and combination vaccines allow for fewer administrations but broad-spectrum protection against several pathogens¹¹. Another class of combination drugs is polyclonal antibodies, which are mixtures of synergistic monoclonal antibodies (mAbs) that simultaneously interact with multiple epitopes either on the same target or on distinct targets¹²⁻¹⁵. For example, ZMapp, an anti-Ebola virus drug, combines three mAbs¹⁶; and the combination of lumiliximab and rituximab showed enhanced antitumor effect in clinical studies¹⁷. Although mAb mixtures have certain advantages, such as synergistic effects and broad-spectrum protection¹⁸⁻²¹, the cost to manufacture them using conventional strategies is much higher than that of producing single mAbs because each mAb needs its own production strain and manufacturing equipment. Thus, strategies for producing multiple mAbs and other biologics in a single batch as a co-culture would be potentially advantageous.

Chinese hamster ovary (CHO) cells are often used for biologics manufacturing²². However, because of their slow growth rate, CHO cells are amenable for on-site, rapid drug manufacturing. K. phaffi is also used as a heterologous protein expression host because it: i) can secrete large amounts of recombinant proteins using the alpha mating factor secretion signal but secretes few host proteins, ii) grows rapidly in inexpensive media, iii) has a eukaryotic post-translational modification system, and iv) is not contaminated with endotoxins or viruses²³⁻²⁵. Furthermore, glycoengineered K. phaffi strains with humanized glycosylation pathways are able to produce recombinant proteins and antibodies with humanized glycosylation profiles^{26,27}. Synthetic biology offers a variety of tools to regulate gene expression in various organisms, including K. phaffi. Recently, our lab developed a recombinase-based gene integration approach enabling the efficient insertion of large DNA fragments into the K. phaffi genome, and an estrogen-inducible promoter, in addition to the native methanol-inducible promoter (AOX1 promoter)⁶. These tools were used to selectively produce one of two different biologics at a time in a portable microbioreactor platform.

Here, we developed a versatile and consolidated bioprocessing platform to further streamline on-demand protein drug production. To explore the manufacturing of therapeutic protein mixtures, we designed three strategies of protein co-expression in K. phaffi: i) a single strain with two inducible expression systems, ii) a single strain with one inducible and one constitutive expression system, and iii) two strains both having the same inducible expression system (Table 8). Instead of producing each biologic separately, each strategy yielded protein mixtures produced as a single batch. We also report on the separation and purification of individual therapeutic proteins from the protein mixtures. Finally, to establish the scalability of our approach, we constructed a third inducible system and showed orthogonal inducible production of three different therapeutic proteins.

TABLE 8
Three strategies for therapeutic protein co-production.

| | No. of strains | Promoter 1 | Promoter 2 | Drug mixtures | Types |
|---|---|---|---|---|---|
| 1 | Single | Methanol | Estrogen | hGH and IFN | Different drugs for |
| | strain | | | | two indications |
| 2 | Single | Methanol | Constitutive | HSA and hGH | HSA associated |
| | strain | | (Estrogen) | | formulation |
| 3 | Multiple | Methanol | Methanol | anti-PD1 and | Antibody mixtures |
| | strains | | | anti-CTLA4 | |

Co-Expressing Two Therapeutic Proteins from a Single Strain with Two Inducible Expression Systems

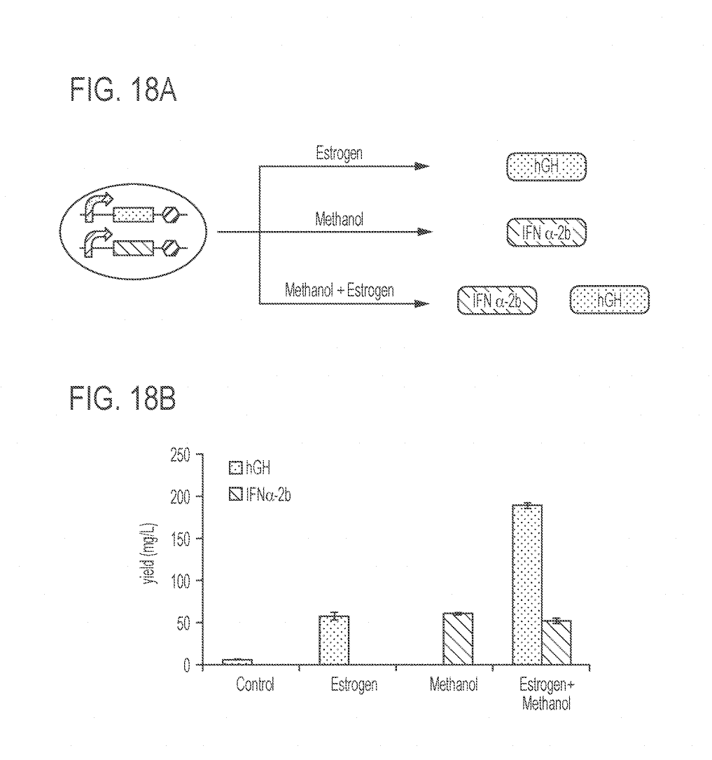

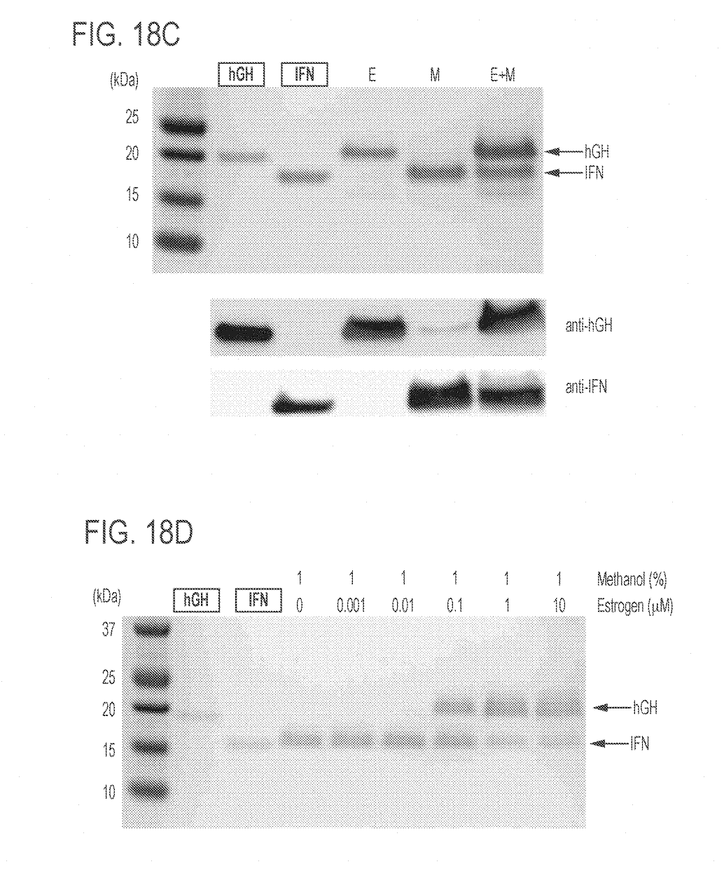

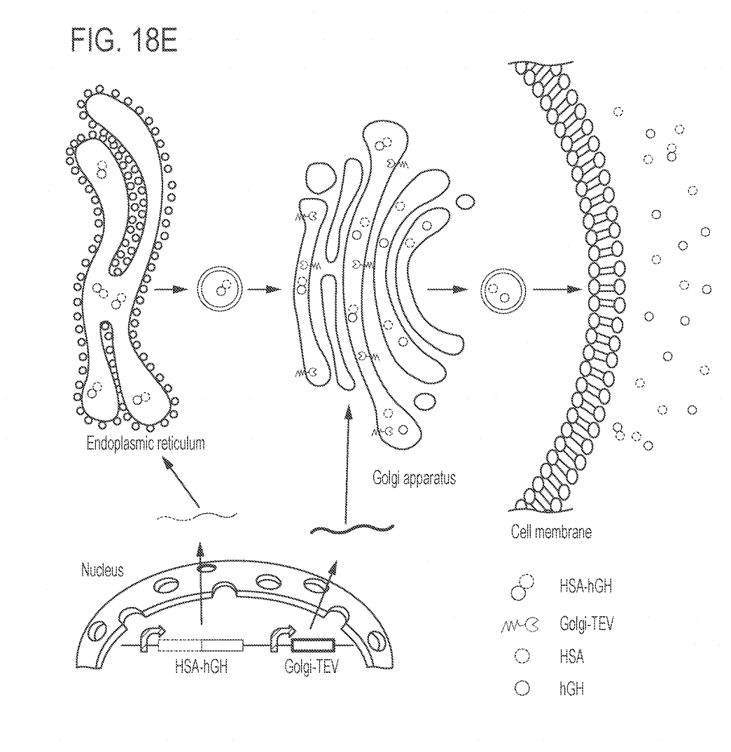

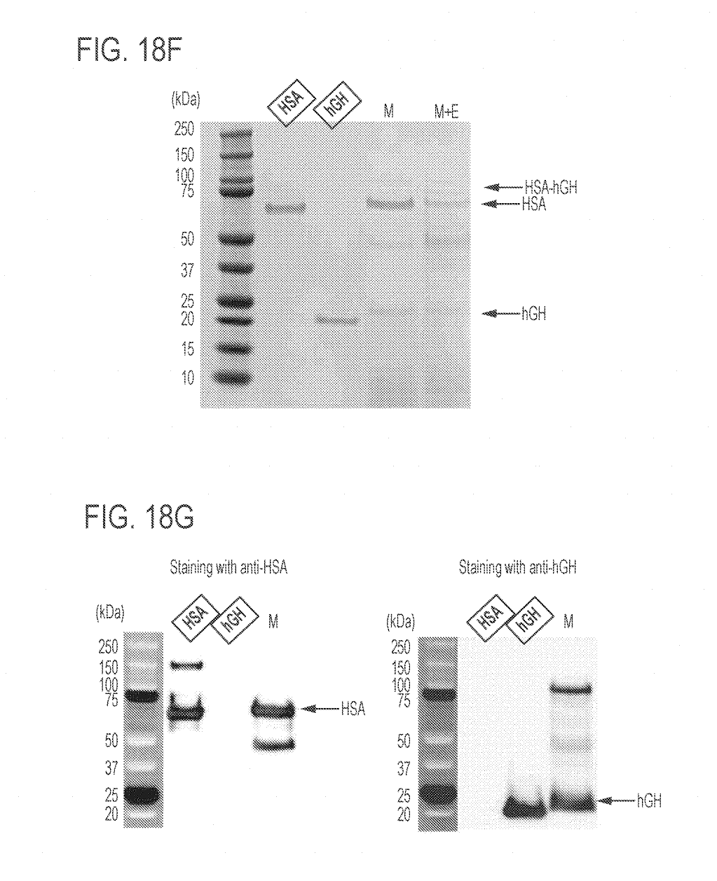

To create a flexible system to produce one or more biologics, we began by constructing a 2-biologics K. phaffi strain (pPP363) that could be programmed to produce human growth hormone alone, interferon alone, or both proteins at once. Human growth hormone (hGH), a 22 kDa therapeutic protein used to treat growth hormone deficiency, was placed under the control of an estrogen-inducible promoter. Interferon α-2b (IFN), a 19 kDa antiviral protein drug, was placed under the control of the AOX1 methanol-inducible promoter⁶. After 48 hours of induction, 58 mg/L hGH was produced in the presence of estrogen, 61 mg/L of IFN in the presence of methanol, and 189 mg/L hGH and 53 mg/L IFN in the presence of both estrogen and methanol (FIG. 18A and FIG. 18B). The results were confirmed by Coomassie blue staining and Western blotting (FIG. 18C).

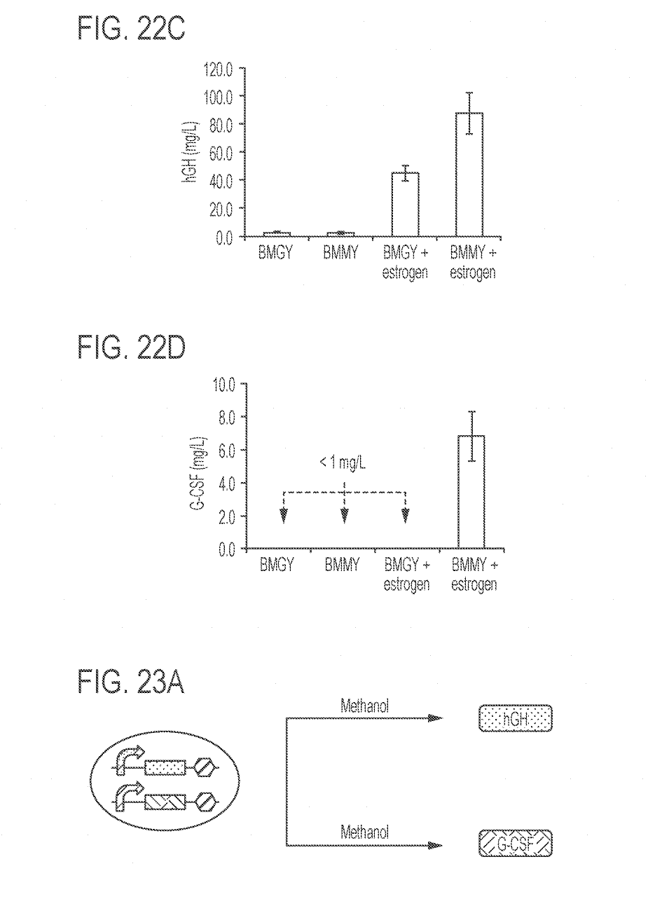

Interestingly, the titer of estrogen-induced hGH significantly increased when hGH and IFN were co-expressed versus the condition where hGH was expressed on its own. To explore this further, we tested whether the use of methanol as a carbon source could enhance the strength of the estrogen promoter or increase protein secretion. We designed three estrogen-inducible protein expression cassettes, one that that expressed intracellular GFP (pPP255) and the other two that secreted either hGH (pPP364) or granulocyte-colony stimulating factor (G-CSF) (pJCO21). We found that estrogen-induced intracellular GFP expression was similar with or without methanol, whereas estrogen-induced hGH and G-CSF secretion increased in the presence of methanol (FIGS. 22A-22D). The results demonstrated that methanol can enhance the secretion of certain proteins in K. phaffi.

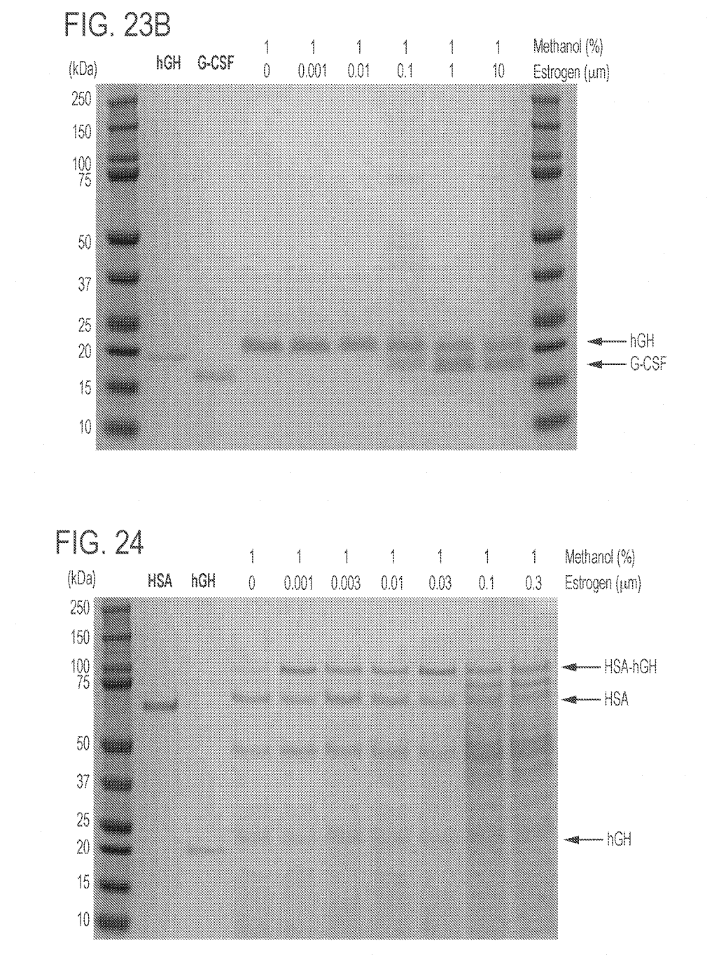

Having established that we could co-express two biologics in a single strain of K. phaffi, we then sought to fine-tune the ratio of the co-expressed proteins with our two inducible systems by varying inducer concentrations during fermentation. The 2-biologics strain (pPP363) was grown for 48 hours and induced with methanol and 0 to 10 μM estrogen. The ratio of hGH to IFN increased as the concentration of estrogen increased (FIG. 18D). To establish the generality of this observation, we constructed another 2-biologics strain (pJC034), with methanol-inducible hGH and estrogen-inducible G-CSF. We observed that the ratio of G-CSF to hGH increased as the concentration of estrogen increased (FIGS. 23A-23B).

When two traditional single-biologic strains are co-cultured, the ratio of the two biologics can be regulated by varying the seeding density of the two strains^{12,28}. However, temperature and pH fluctuations during fermentation can change the growth rates of the strains, making ratio control solely based on seeding density challenging²⁹. In contrast, our 2-biologics strategy enables dynamic control over the ratios between two biologics via inducer concentrations without needing to modulate strain growth rates.

Consolidated Post-Translational Bioprocessing with Two Expression Systems

The formulation of unstable proteins is difficult, especially for hydrophobic proteins, such as growth factors, interferons, and cytokines³⁰. To enhance solubility and reduce drug adsorption on the surface, excipients are used to formulae drugs. One excipient used in the pharmaceutical industry is human serum albumin (HSA), the most abundant protein in human plasma. HSA, which can also be used as a drug, has a low risk of immunogenicity and stabilizes proteins by reducing aggregation, oxidation, and nonspecific adsorption³¹⁻³³. However, the addition of another established cell line and manufacturing platform to produce HSA can make it costlier than other small-molecule excipients (e.g., sugars, amino acids, and surfactants). Therefore, we envisioned that co-expressing a protein drug (hGH) along with HSA as an excipient in a single engineered K. phaffi could address the problem.

K. phaffi can effectively secrete large amounts of recombinant HSA and HSA fusion proteins³⁴⁻³⁶. We constructed a strain expressing two fusion proteins (pJC171): i) HSA-hGH consisting of an alpha-mating factor secretion signal, HSA, a tobacco etch virus (TEV) protease cleavage site, and hGH; and ii) Golgi-TEV, consisting of a Golgi apparatus localization signal (the membrane-binding domain of alpha-1,2-mannosyltransferase) and TEV protease^{26,37}. TEV protease recognizes the amino acid sequence ENLYFQ/X (SEQ ID NO: 30) and cleaves between glutamine (Q) and X (P1′ site amino acid), where X can be any amino acid except proline (P)^{38,39}. This feature of TEV makes it a widely used protease to produce intact proteins from fusion proteins⁴⁰. We envisioned that the fusion protein HSA-hGH would be synthesized and folded in the endoplasmic reticulum (ER) and then would enter the Golgi before being secreted. The Golgi localization signal should direct the localization of TEV protease to the inner membrane of the Golgi, where it cleaves the ready-to-be-secreted HS A-hGH into HSA and intact hGH (FIG. 18E). Although 2A peptides have been used to secrete multiple proteins from a single cistron at the translational lever, our approach provides a new strategy to produce multiple biologics at the post-translational level with only a single secretion signal.

We observed that the overexpression of intracellular TEV protease lysed the cells (FIG. 18F and FIG. 24), so we tuned estrogen-induced TEV protease expression with estrogen and used the methanol-inducible promoter to express HSA-hGH. Our dose-response experiments revealed that basal expression of TEV protease was sufficient for effective cleavage, whereas induction of TEV expression with estrogen at a higher concentration (0.1 μM) caused cell lysis. Thus, we induced HSA-hGH expression with methanol and allowed TEV protease to be constitutively expressed, LISA-hGH was correctly cleaved by basally expressed TEV, yielding HSA and hGH, as verified by Coomassie blue staining (FIG. 18F) and Western blotting (FIG. 18G). We also observed some uncleaved fusion protein, which could be explained by previously studies that showed that the processing efficiency of TEV protease is 90% when phenylalanine (F) occupies the P1′ site, since phenylalanine is the N-terminal amino acid of hGH³⁹.

The uncleaved fusion protein could be removed together with cell host proteins using traditional chromatography if needed. Our system is thus able to achieve consolidated bioprocessing of therapeutic proteins at the post-translational level. This strategy could be potentially adapted to regulate other post-translational processes, such as glycosylation, by replacing the TEV protease with glycosyltransferases and glycan-processing enzymes.

Single Batch Manufacturing of Biologics Mixtures with Two Strains Containing the Same Inducible Expression System

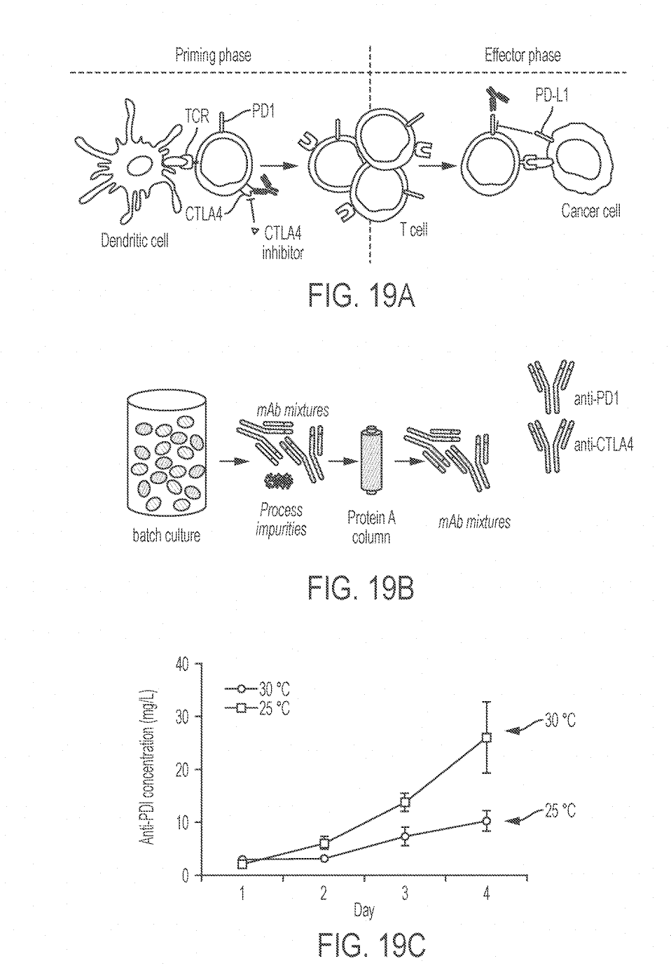

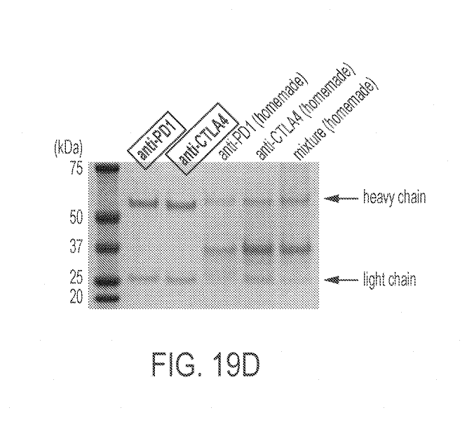

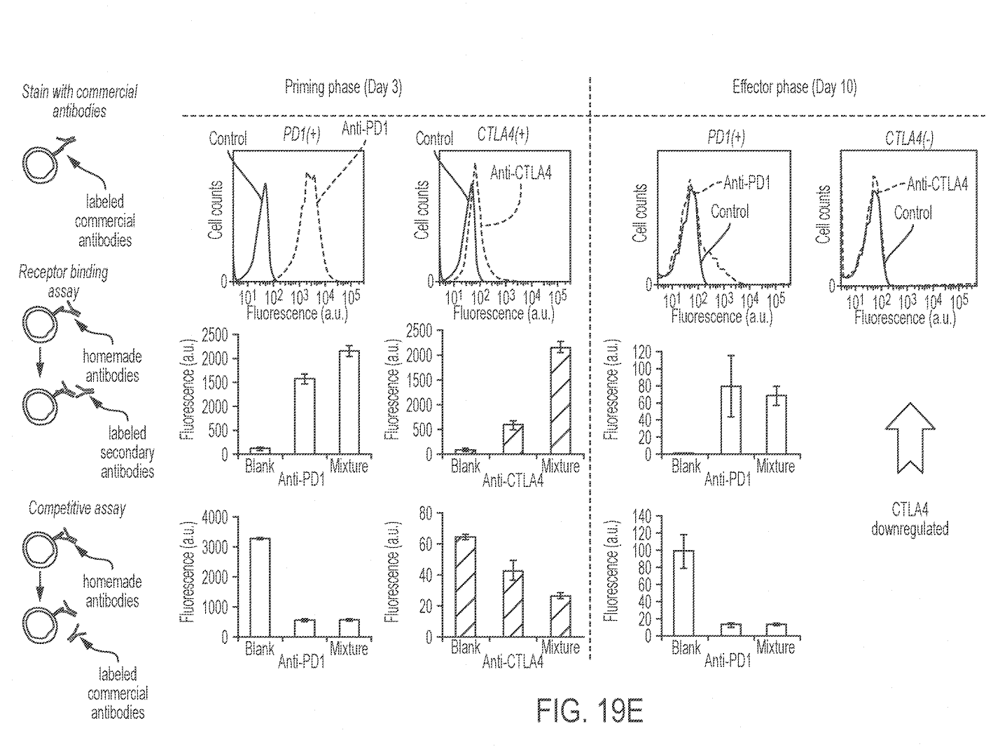

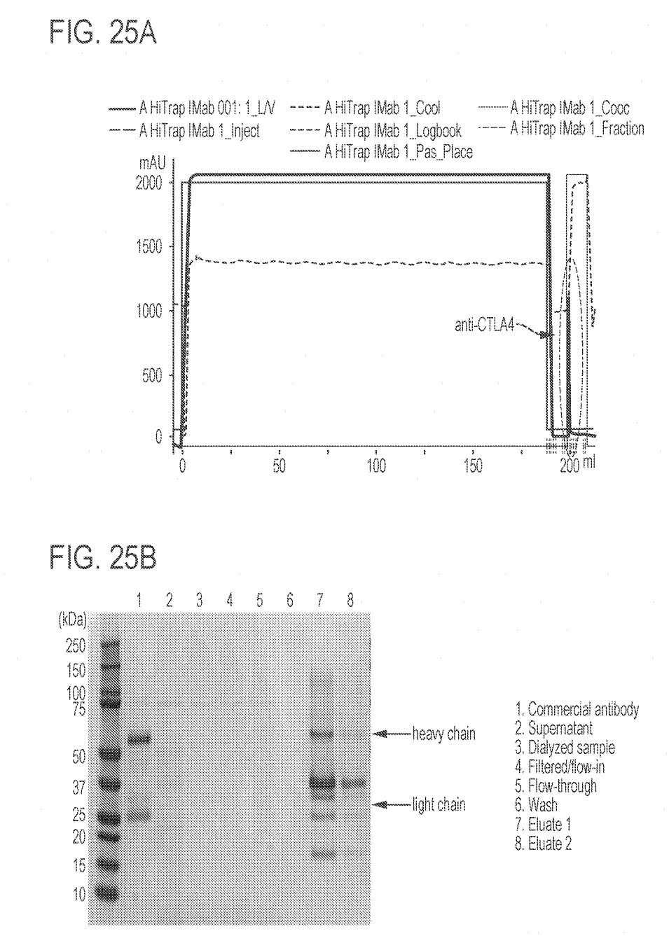

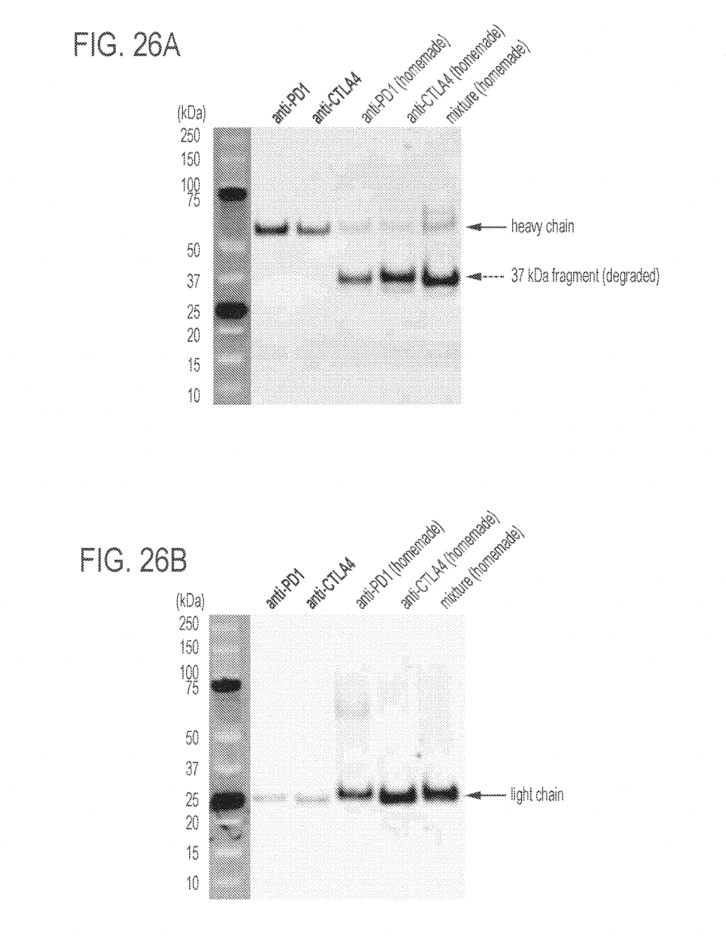

Traditionally, polyclonal antibodies are made by producing each mAb separately and mixing the purified mAbs to make the final products. It was previously shown that the manufacturing cost for a mixture of two antibodies is about double that for a single mAb using conventional approaches^{12,13,28}. We sought to co-culture two strains together to produce antibody mixtures within a single batch, thereby reducing manufacturing costs. To demonstrate a relevant proof of concept, we chose a mixture of two therapeutic antibodies, anti-cytotoxic T-lymphocyte-associated antigen 4 (anti-CTLA) and anti-programmed death 1 (anti-PD1). Both are checkpoint inhibitor antibodies approved for treating advanced melanoma^{18,21}. The targets of these antibodies, CTLA4 and PD1, respectively, both negatively regulate T cells, but they are upregulated at different stages of T-cell activation. CTLA4 is briefly upregulated in the priming phase whereas PD1 is consistently expressed in the effector phase of T cell activation.^{42,43} The human anti-CTLA4 antibody binds to CTLA4 on the T-cell surface, blocking CTLA4 from shutting down T-cell activation in the early stage, and the human anti-PD1 antibody binds to PD1, preventing tumor cells from inhibiting T-cell activity (FIG. 19A). We constructed two K. phaffi strains that each produced one of the mAbs (pJC110 expressing anti-PD1 antibodies and pJC111 expressing anti-CTLA4 antibodies) and optimized culture conditions (temperature and time) for antibody production (FIGS. 19B and 19C). We produced mixtures of these two antibodies by co-culturing the two strains. The antibodies were purified using protein G column (FIGS. 25A-25B) and then verified using SDS-PAGE (FIG. 19D) and Western blotting (FIGS. 26A-26B).

To test the activity of these antibodies, we assayed cell surface receptor binding on human primary T cells. Human primary T cells were activated with phytohaemagglutinin (PHA) to express the cell surface receptors PD1 and CTLA4. On Day 3 and Day 10 post-induction, we analyzed the expression of the receptors using commercial anti-PD1 and anti-CTLA4. On Day 3, almost 99% of the activated T cells were expressing PD1 and 15% of them were expressing CTLA4, consistent with prior studies (FIG. 19E)^{42,43}.

We then used cell binding assays and a competitive assay to confirm the correct structures and targets of the antibodies produced in K. phaffi. Purified anti-PD1 antibody alone, anti-CTLA4 antibody alone, and the mixture of these co-produced two antibodies made in this study were added to the cells, and then stained with labeled detection antibodies. Antibodies in all three samples bound to the activated T cells (FIG. 19E). Competitive assays with commercial antibodies binding to the two receptors were also performed to confirm that the two homemade antibodies produced in K. phaffi did indeed bind to their respective targets. We first incubated the cells with either homemade anti-PD1 or the mixture, and then incubated the cells with PE-labeled commercial anti-PD1. The fluorescence of the cells incubated with homemade anti-PD1 and then incubated with PE-labeled commercial anti-PD1 decreased compared to that of the cells incubated with only PE-labeled commercial anti-PD1, indicating that the homemade antibody bound to the same epitope as the commercial anti-PD1 (FIG. 19E). The same assay for our anti-CTLA4 antibody showed that this antibody bound to CTLA4 (FIG. 19E).

On Day 10, the activated T cells are expected to be in the effector phase, when CTLA4 expression is downregulated but PD1 expression is maintained. Using commercial antibodies, we observed the expression of PD1 and the disappearance of CTLA4 staining (FIG. 19E). Using homemade anti-PD1 antibodies and the antibody mixture, we then confirmed the blocking of PD1 receptors (FIG. 19E). These results indicate that the co-culture and co-purification of the antibody mixture in a single batch in K. phaffi could simplify the manufacturing process for antibody mixtures. Compared with mammalian hosts, the use of K. phaffi has the potential to decrease the time and cost needed to produce antibodies and antibody mixtures. Moreover, the ratio of two antibodies should be tunable if we replaced the AOX1 promoter of one strain with the independently inducible estrogen promoter.

Selective Separation of Individual Therapeutic Proteins from Biologics Mixtures

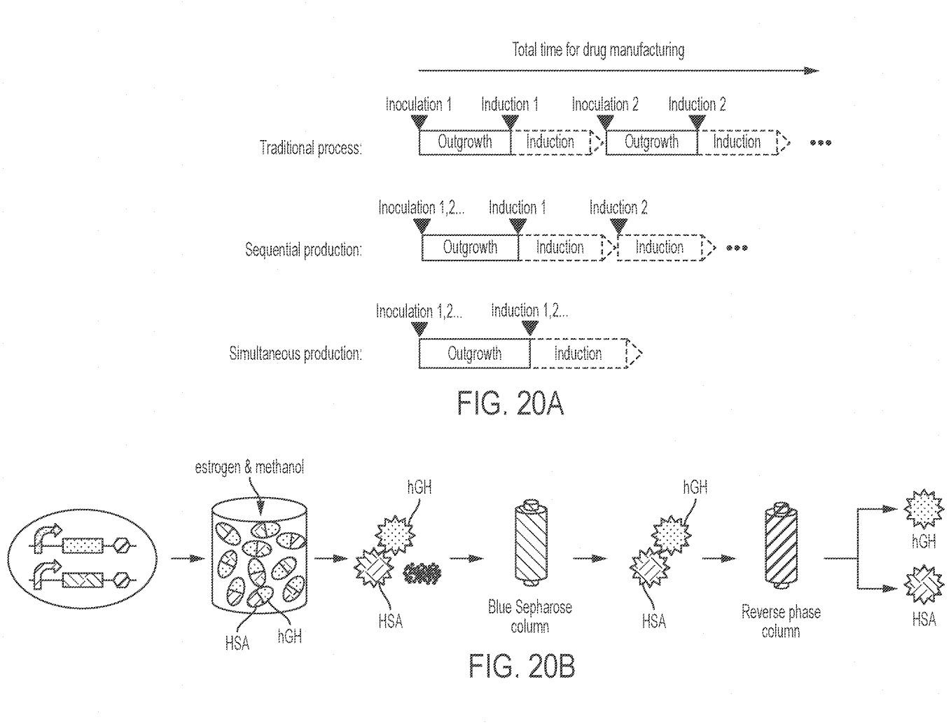

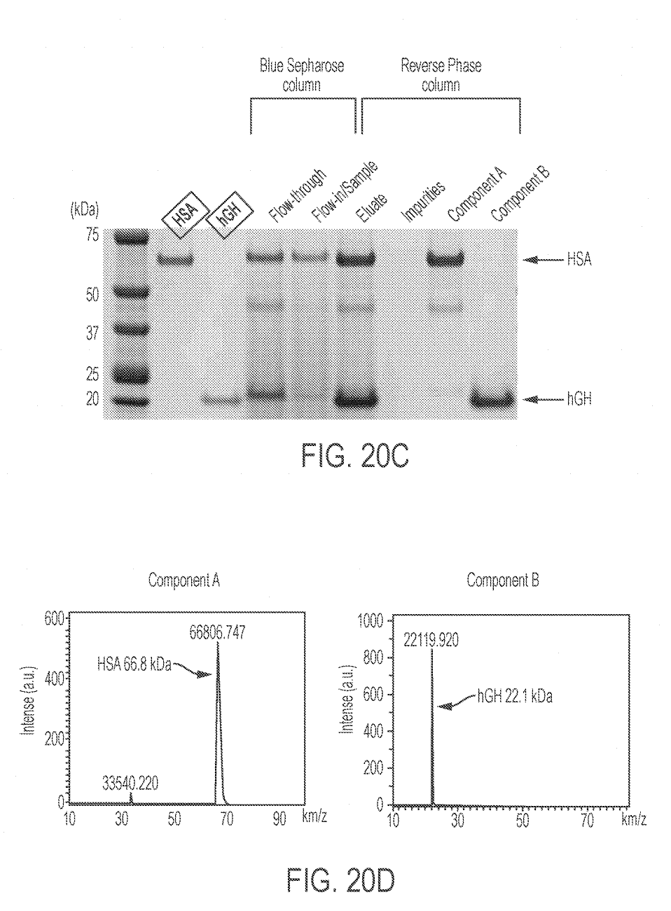

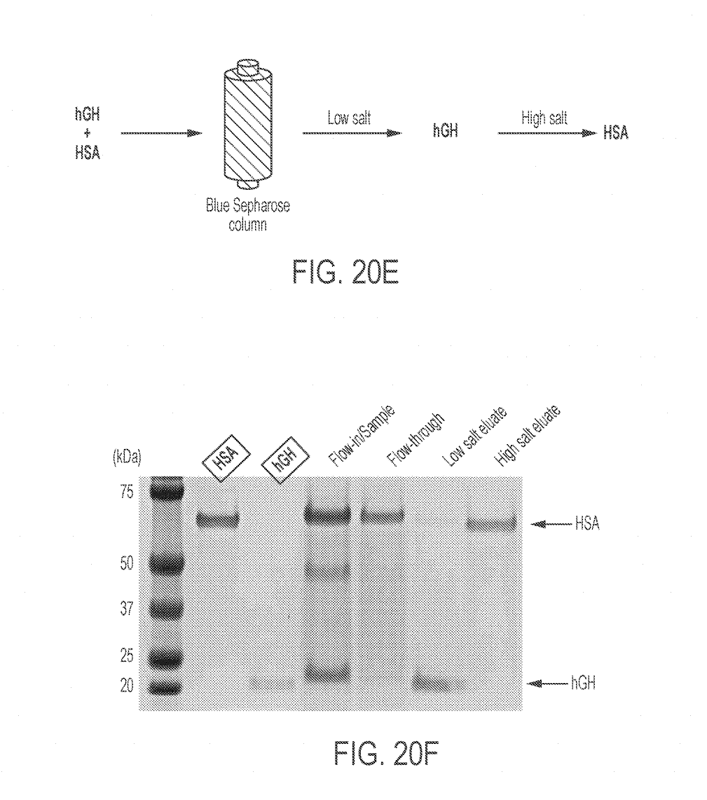

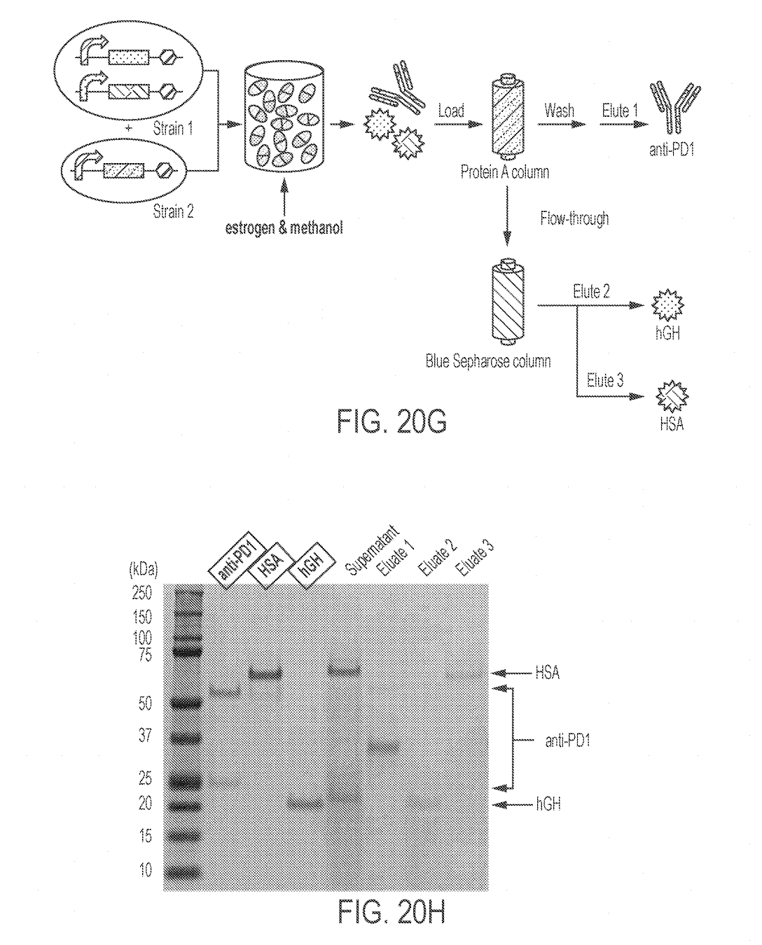

Having established 3 effective methods to produce multiple biologics in a single batch, we sought to develop purification procedures that could be used to separate out individual therapeutic proteins from these mixtures. It is economically difficult to have multiple parallel manufacturing platforms to produce different drugs where resources are scarce. To make multiple drugs in small quantities with only one set of manufacturing equipment, we sought to generate mixtures of biologics and then separate them through downstream processing. We expected this co-production-plus-separation methodology to take less time than existing procedures (FIG. 20A). We previously showed that we could produce two therapeutic proteins sequentially in a single manufacturing platform, thus reducing the total manufacturing time from (t_{growth}+t_{induction})×2 to (t_{growth}+t_{induction}×2)⁶, where t_{growth} refers to the amount of time needed to grow the production host to high cell densities and t_{induction} refers to the amount of time needed to induce expression of the desired drug. For example, it takes about four days to produce one protein in Pichia: 2 days to grow the strain (t_{growth}) and 2 days to express the protein (t_{induction}). Thus, it would take 6 days to express two proteins from a single strain if one grew the strain and then induced it sequentially to make each of the proteins in succession. Here, we aimed to further reduce the time to produce for N proteins from (t_{growth}+t_{induction}×N) with sequential induction to (t_{growth}+t_{induction}) with simultaneous manufacturing (FIG. 20A). For example, if one expressed the two proteins simultaneously, it would only take 4 days: 2 days to grow the dual-biologics strain and 2 days to express the two proteins. Downstream separation and purification should require from several hours to 1 day in all strategies. We used HSA and hGH as examples to demonstrate a prototypical workflow for the proposed simultaneous production strategy.

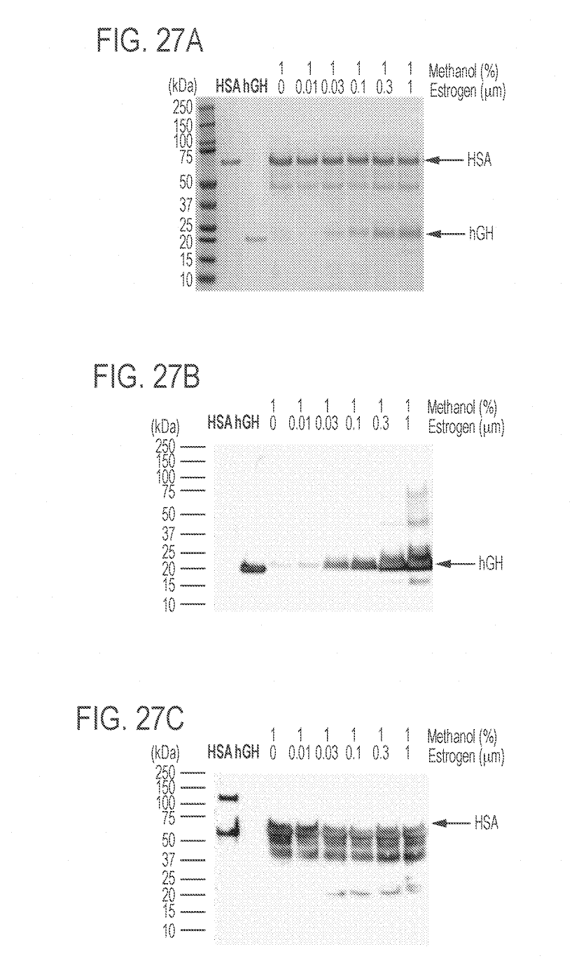

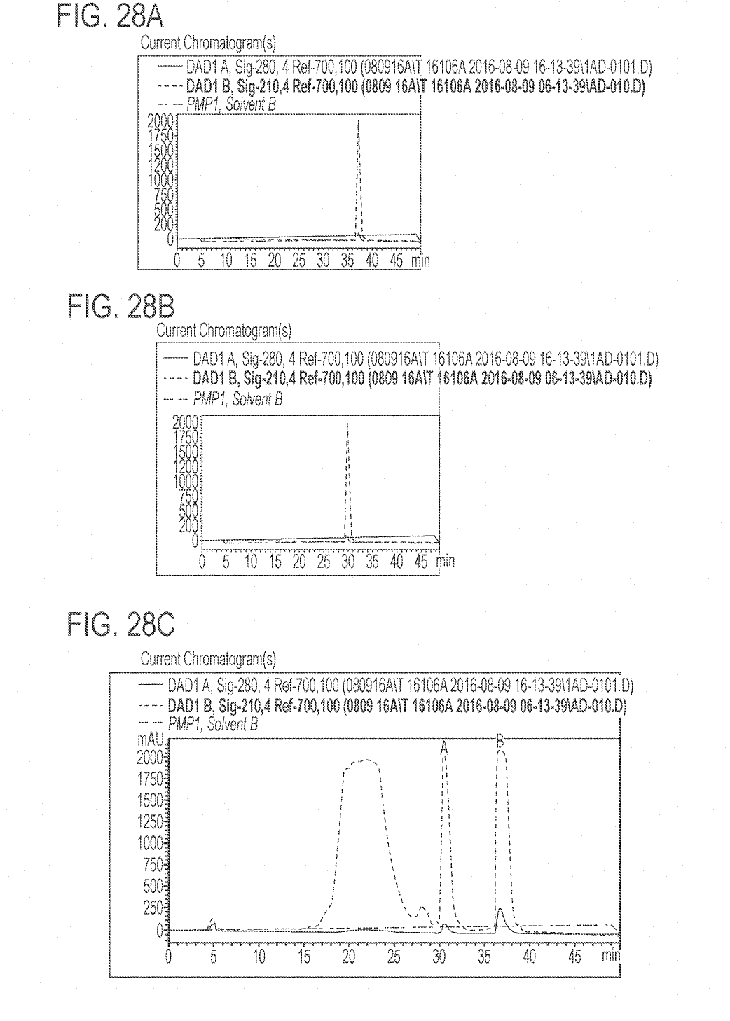

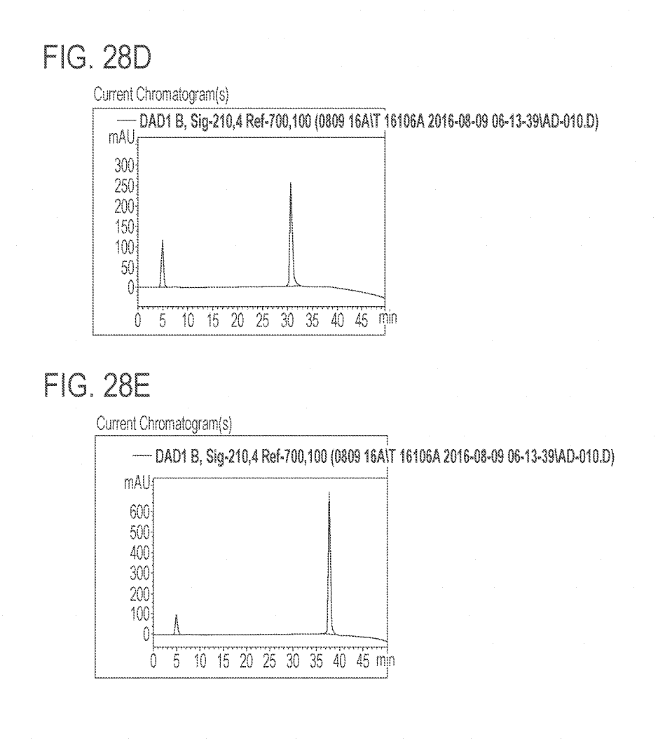

We first purified proteins produced by the single strain expressing HSA upon methanol induction and hGH upon estrogen induction (pJC135) with two inducible expression systems. We engineered this system so that the methanol-inducible promoter made USA and the estrogen-inducible promoter generated hGH (FIG. 20B). The co-expression of HSA and was confirmed by SDS-PAGE and Western blotting, and the ratio of hGH to HSA could be tuned by varying the concentration of estrogen (FIGS. 27A-27C). To purify HSA and hGH in the supernatant, we used a Blue Sepharose column, which binds a variety of proteins, including albumin, interferon, lipoproteins, blood coagulation factors, and several enzymes (FIG. 20B)^{34,44}. We loaded the supernatant into the column, and eluted hGH and HSA with high salt buffer to get rid of most of the host cell proteins. The resulting eluate was further purified using reverse-phase chromatography, and the peaks of hGH and HSA were collected (FIGS. 28A-28E). The samples were then analyzed by using SDS-PAGE gel and Matrix-assisted laser desorption/ionization (MALDI) (FIG. 20C and FIG. 20D). MALDI chromatographs indicated that the separation of hGH and HSA was virtually complete (below the detection limit). Our two-step purification strategy used a Blue Sepharose column (column 1) for purifying the two proteins from the host proteins and a reverse-phase column (column 2) to separate the two proteins.

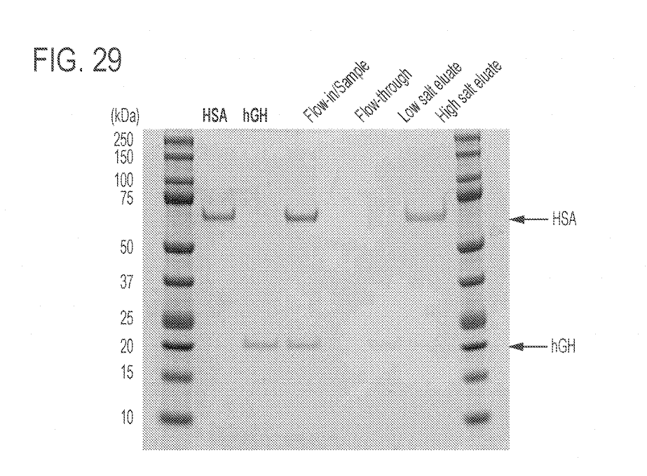

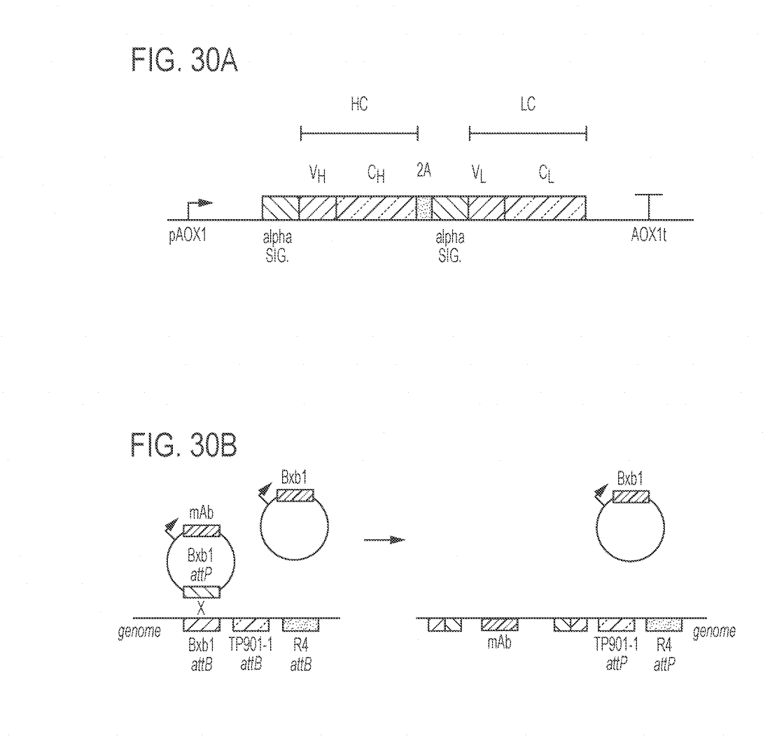

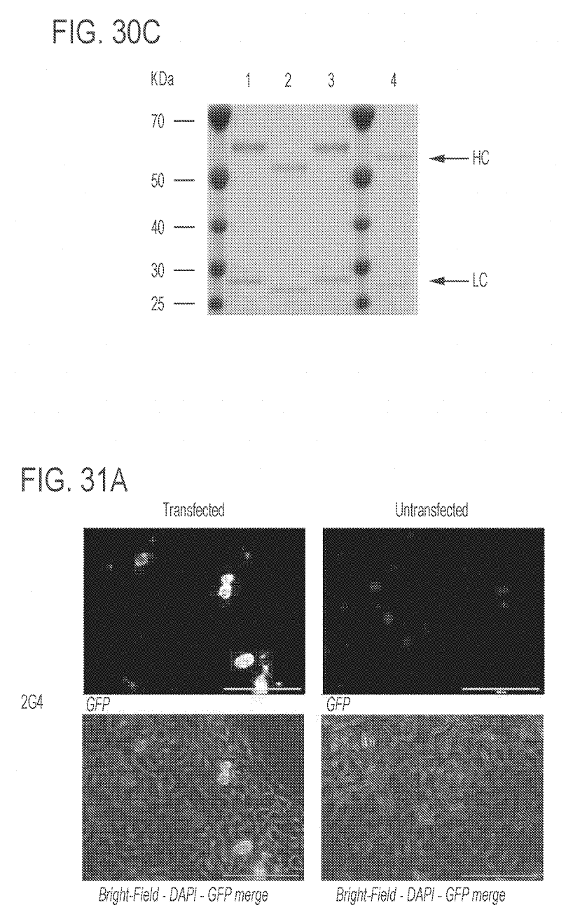

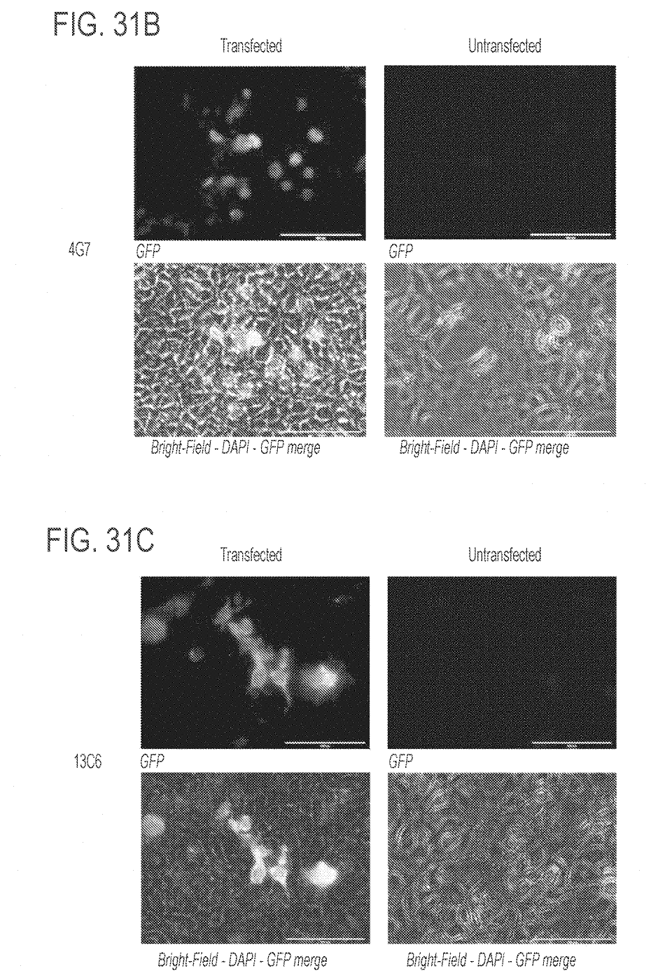

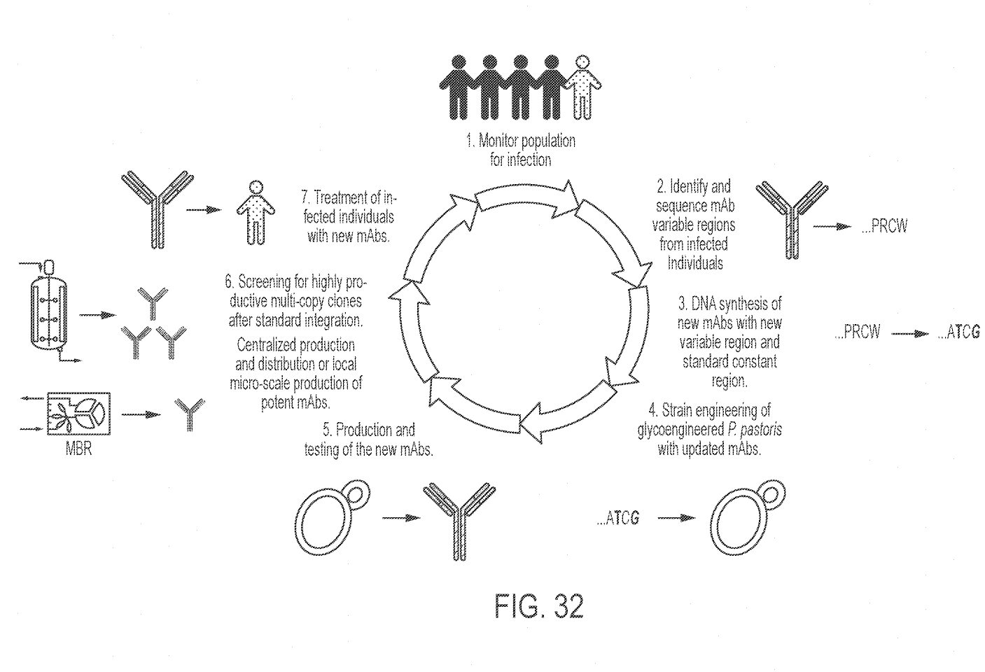

To simplify purification further, the number of columns used for the separation of HSA and hGH was reduced based on the idea that proteins with different binding affinities to the Blue Sepharose column can be eluted with different elution conditions, such as salt concentration. We tested various conditions using commercial HSA and hGH samples and found that a low salt buffer (20 mM sodium phosphate and 100 mM sodium chloride) could be used to elute hGH and that a high salt buffer (20 mM sodium phosphate and 2000 mM sodium chloride) could be used to elute HSA (FIG. 20E and FIG. 29). We then used the same strategy to demonstrate the separation of HSA and hGH in the supernatant (FIG. 20F). The fraction eluted first contained 92.4% hGH and 7.6% HSA, whereas the second eluate contained 95.4% HSA and 4.6% hGH, which was calculated using ImageJ. If drugs of high quality are required for further testing or clinical use, minor components and other impurities can be removed through traditional chromatography purification processes.

To further multiplex this approach, we sought to combine multiple protein co-expression strategies. We co-cultured two strains, one strain expressing HSA upon methanol induction and hGH upon estrogen induction (pJC135), and one strain expressing anti-PD1 antibody (pJC110) upon methanol induction.

Ninety-six hours post-induction, the supernatant containing HSA, hGH, and anti-PD1 was harvested and dialyzed against 20 mM sodium phosphate. We chose two commercially available columns for separation: a Protein A column was used for antibody purification, as the Fc region of antibodies binds to protein A at neutral pH and can be eluted at low pH (pH=3.0); and a Blue Sepharose column was used to separate hGH and HSA, as described above. To separate the three proteins, the supernatant was first injected into a Protein A column Anti-PD1 was captured in the column whereas hGH, HSA, and the cell host proteins passed through. Anti-PD1 was then eluted by using a low pH buffer. The flow-through was then injected into the Blue Sepharose column hGH and HSA were captured in the column whereas the cell host proteins passed through. hGH was eluted with low salt buffer and HSA was then eluted with high salt buffer (FIG. 20G and FIG. 20H). The fraction eluted first contained 86.1% hGH and 13.9% HSA, whereas the later eluate contained 89.9% HSA and 10.1% hGH, which was calculated using ImageJ. Thus, we achieved primary recovery and effective separation of individual drugs from co-expressed drug mixtures, which can be followed by traditional chromatography purification processes for clinical studies.

### Construction of a Third Inducible System for Orthogonal Control of Three Therapeutic Proteins

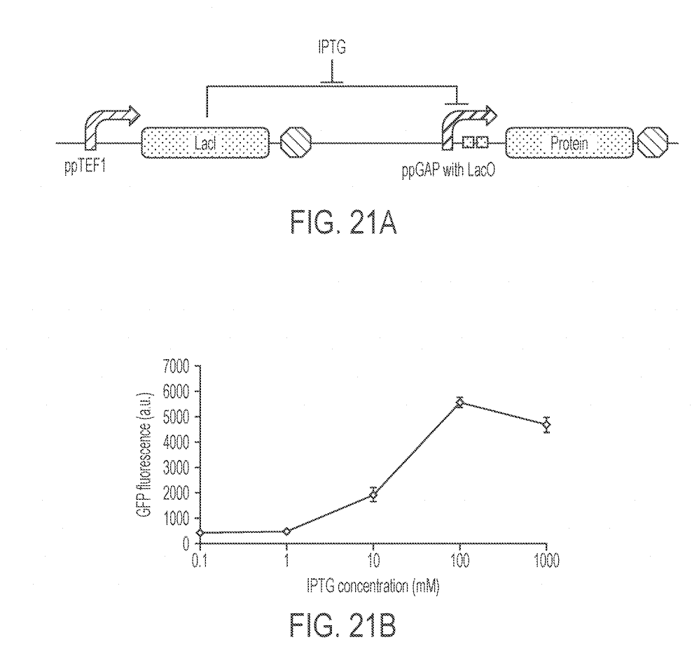

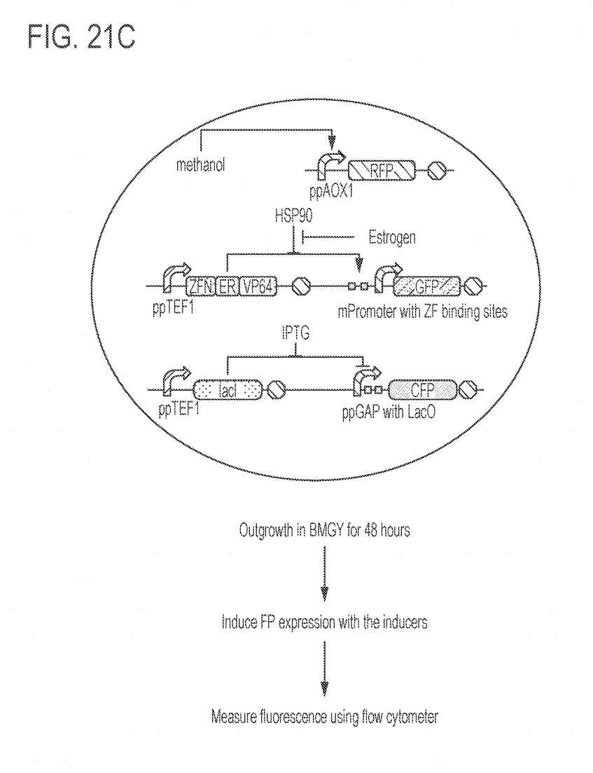

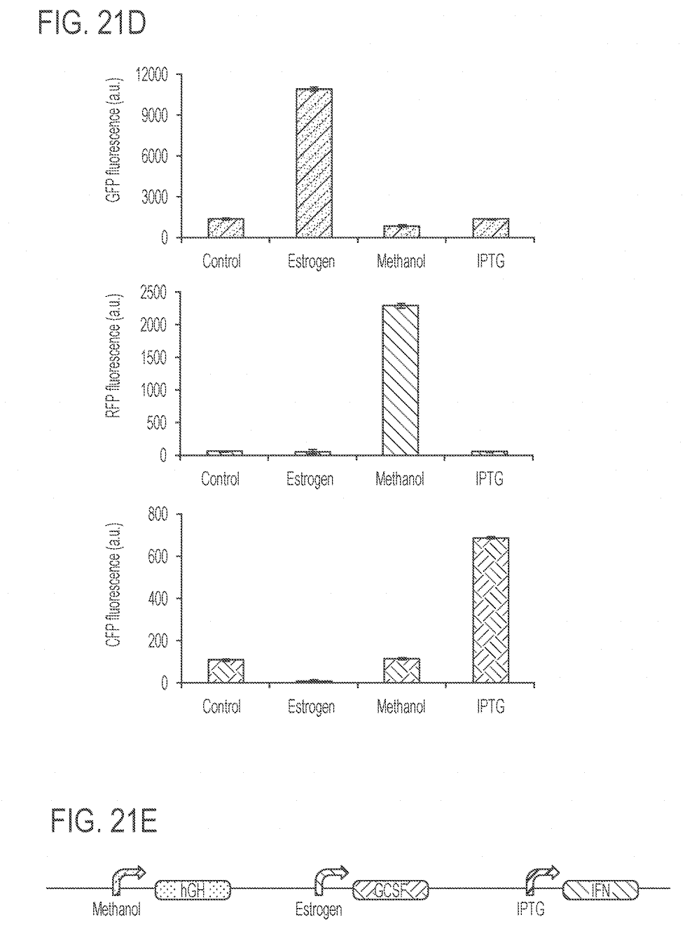

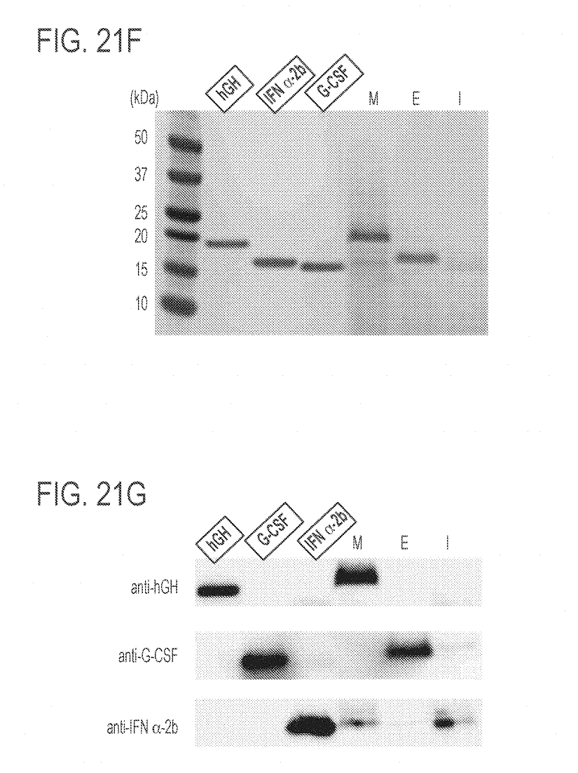

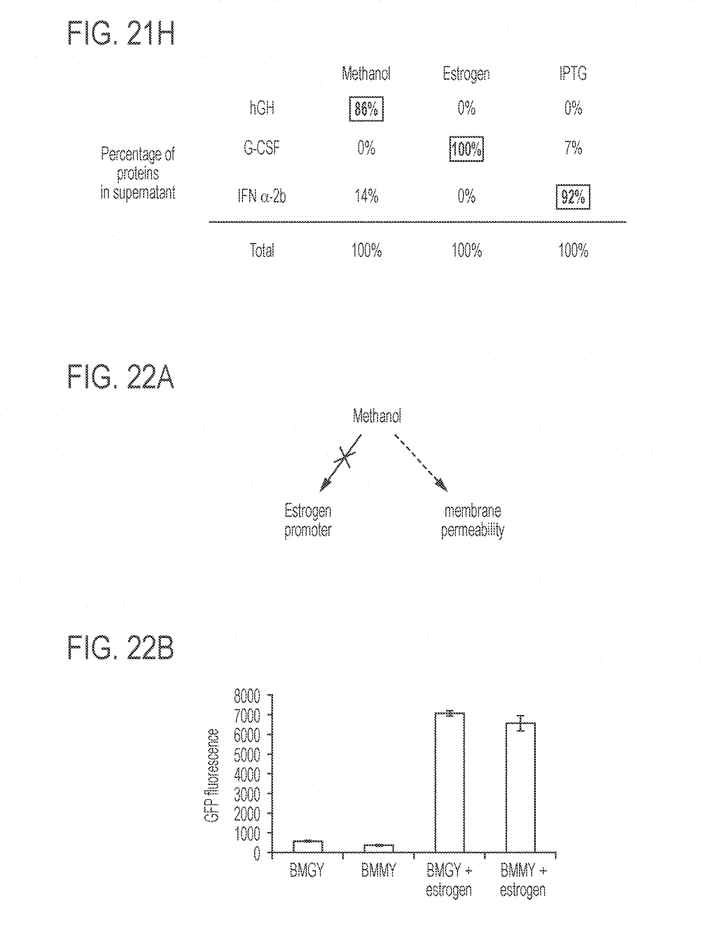

To demonstrate the potential scalability and generality of this approach, we designed a third inducible gene expression system in K. phaffi that was inducible with IPTG (isopropyl β-D-1-thiogalactopyranoside). We inserted two lac operator (lacO) sequences next to the GAP constitutive promoter, and used constitutive TEF1 to drive the expression of lac repressor (LacI). Lad repressor proteins bind to the lac operator on the GAP promoter in the absence of IPTG, thus preventing RNA polymerase from binding and transcribing from the artificial GAP promoter. IPTG releases Lad from the promoter, initiating transcription (FIG. 21A)^{45,46}. We used GFP as the reporter, and constructed a K. phaffi strain carrying the IPTG-inducible system (pPP309). In a dose-response test, GFP fluorescence was activated six-fold in the presence of 1 mM IPTG compared to no IPTG, validating the inducibility of this system (FIG. 21B).

We then tested the orthogonality of the three systems (methanol-inducible, estrogen-inducible, IPTG-inducible) by integrating a plasmid consisting of methanol-inducible RFP, estrogen-inducible GFP, and IPTG-inducible CFP protein expression cassettes into the K. phaffi genome (pJC101) (FIG. 21C). We induced protein expression with the respective inducers and measured fluorescence intensity by flow cytometry after 48 hours. We observed expected inducible gene expression and found that there was no cross-activation between the three inducers and the non-cognate promoters (FIG. 21D).

Having demonstrated the selectivity and orthogonality of the three inducible systems in K. phaffi, we sought to produce the therapeutic proteins hGH, G-CSF, and IFN. We used the methanol-inducible promoter to express hGH, the estrogen-inducible promoter to express G-CSF, and the IPTG-inducible promoter to express IFN (FIG. 21E). G-CSF was not stable in the medium, so we added protease inhibitors to increase its expression (FIG. 21F). The therapeutic proteins were validated and quantified by Western blotting (FIG. 21G). The titer of hGH was 51.2 mg/L (86% of the total therapeutic proteins) in the presence of methanol; that of G-CSF was 22.9 mg/L (100% of the total therapeutic proteins) in the presence of estrogen; and that of IFN was 9.5 mg/L (92% of the total therapeutic proteins) in the presence of IPTG (FIG. 21H). We observed IFN expression in media with methanol, but did not observe CFP expression from the same IPTG promoter (FIG. 21D), consistent with the hypothesis that methanol can enhance certain protein secretion but not intracellular protein expression (FIGS. 22-A-22D).

### SUMMARY

We have developed flexible and consolidated bioprocessing schemes for integrated rapid strain engineering, inducible protein expression, and selective or combined protein purification. We showed simultaneous production of multiple biologics and combination drugs by integrating inducible protein expression systems with upstream and downstream bioprocessing in K. phaffi. We demonstrated inducible expression of single biologics, simultaneous production of multiple distinct biologics, co-production of protein mixtures, and ratio control for combinations. We also present a single-batch approach for polyclonal antibody production, which can be used for cancer immunotherapy and other therapeutic applications. Finally, we constructed a system that allows orthogonal triple-gene control of the inducible production of three therapeutic proteins. This system described in the work can produce one, multiple, or combination proteins at a defined ratio from one strain of K. phaffi and with one set of production equipment in a short timeframe. The ability to produce multiple therapeutic proteins simultaneously in a single batch has the potential to significantly reduce the number of strains and facilities required for protein production, thus lowering time and expense.

Previously we developed a portable device to produce a single dose of two different drugs at the point-of-care, which can be used to provide medication for people at remote areas or to prevent pandemics⁶. In a continuous manufacturing mode, such as perfusion culture, we can consistently produce a protein for a long period of time. Although this or other well-established on-demand strategies can manufacture a single type of drug⁴⁻⁶, additional production devices and additional cost and time are required if multiple drugs are needed for the same patient or different patients¹³. Thus, using existing approaches, a choice has to be made between cost (using multiple devices together) and time (producing one drug at a time), both of which increase as the number of regions to be serviced and the number of people to be treated expand, because of the likelihood of concurrent needs for different drugs.

Our platform is suited not only to single drug production, but also the small-scale production of combination drugs (FIGS. 19A-19E) and multiple distinct drugs (FIGS. 20A-20H) at a time. Drugs can be generated as they are needed by adding the corresponding inducers during batch or continuous culture and changing the types and concentrations of inducers dynamically to meet the fluctuating demand for drugs in a certain region, for preclinical studies, or for clinical trials. Compared with the co-culture of different strains, our single-strain production strategy is able to produce one or more desired proteins in the same batch, and the ratio can be dynamically tuned by varying inducer concentrations (FIGS. 18A-18G). The ability to produce mixtures of proteins could be used to enable combination drugs or polyvalent vaccines, or be used with separation technologies to create several distinct drugs for different patients.

When multiple biologics are produced in a single facility but are not used together as combination drugs, there is the risk of cross-contamination. This risk depends on the type of the drug, and can be evaluated using acceptable daily exposure (ADE) values⁴⁷. Recently, Carver proposed a banding scheme to assess the potency or toxicity of biologics; the biologics were categorized according to their toxicity⁴⁸. In this scheme, toxins have the lowest ADEs, and growth factors and antibodies have higher ADEs. Unlike traditional purification processes, our approach consists of two stages: separation and polishing, where one column is used to separate the protein mixture. For example, protein A columns are commonly used for antibody purification, but in our work we used it for both antibody purification and the separation of antibodies and two other proteins (HSA and hGH). The purpose of separation is to maximize the recovery rate of the biologics, while the polishing step purifies the main component by removing other components and processing impurities. In this work, we demonstrated primary separation of antibodies, hGH, and HSA using affinity columns. These molecules can be further purified by traditional processes to remove other components below their ADE levels.

Compared with other small-scale or flexible manufacturing systems⁴⁻⁶, one advantage of our approach is that it can be operated in existing drug manufacturing processes used in academia or industry. For example, our multiple-biologics strains can be grown in common bioreactors and the expression of proteins of interest can be regulated using chemical inducers. Protein mixtures can be separated and polished by adding a commercially available separation column in the purification system, which is ideally the first column to maximize recovery and purity. Protein purification systems usually consist of multiple types of chromatography and filtration, such as affinity chromatography, ion exchange chromatography, and hydrophobicity chromatography to remove impurities (mostly host cell proteins) of various characteristics and obtain high quality products. Protein mixtures can be separated using one or more columns depending on the protein characteristics. Instead of developing new affinity columns or adding tags to the proteins, we can adapt common chromatography columns to purify protein mixtures of interest.

We have constructed three orthogonal inducible systems and developed three strategies for protein co-production. The systems and modes can both be multiplexed to meet the need for customized medications. Additional inducible systems can be designed and advanced genetic circuits can be integrated to increase the number of outputs. For example, adapting this system to utilize non-chemical inducers, such as distinct wavelengths of light, may enhance its utility. If developed as a continuous production system^{6,49}, our platform should be able to produce desired proteins on demand in a dynamic fashion, reducing cost and allowing for precise control over the quantities and relative concentrations of the proteins obtained. Thus, we envision that this platform can reduce the time and cost for producing multiple drugs and improve access to important biologics.

### Materials and Methods

### Media and Buffers

BMGY medium contained 1% yeast extract (VWR, PA), 2% peptone (VWR, PA), 100 mM potassium phosphate buffer (pH=6.0) (VWR, PA), 4×10⁻⁵% biotin (ThermoFisher, MA), 1.34% Yeast Nitrogen Base (Sunrise Science, PA), and 2% glycerol (VWR, PA). BMMY contained 1% yeast extract, 2% peptone, 100 mM potassium phosphate buffer (pH=6.0), 4×10⁻⁵% biotin, and 1% methanol (VWR, PA). YPD contained 1% yeast extract, 2% peptone, and 2% glucose (VWR, PA).

Binding buffer for Protein A, Protein G, and Blue Sepharose columns contained 20 mM sodium phosphate (pH=7.0) (Teknova, Calif.). Elution buffer for Protein A and Protein G columns contained 0.1 M citric acid (pH=3.0) (VWR, PA). Elution buffer for Blue Sepharose column contained 20 mM sodium phosphate and 100 mM sodium chloride or 2000 mM sodium chloride (VWR, PA).

### Strains and Plasmid Construction

The construction of the parental P. pastoris (K. phaffi) strain, derived from wild-type P. pastoris (K. phaffi) strain (ATCC 76273), was described before⁶. The multiple constructs used in these experiments were built using restriction enzyme cloning and/or Gibson assembly. Plasmids are available for distribution at Addgene.

### Electroporation

Competent cells were prepared by first growing a single colony of P. pastoris (K. phaffi) in 5 mL YPD at 30° C. for 48 hours. 100 μL of the resulting culture was inoculated in 50 mL of YPD and grown at 30° C. for another 24 hours. The cells were centrifuged at 1,500 g for 5 min at 4° C. and resuspended in 50 mL of ice-cold sterile water, then centrifuged at 1,500 g for 5 min at 4° C. and resuspended with 20 mL of ice-cold sterile water, then centrifuged at 1,500 g for 5 min at 4° C. and resuspended in 10 mL of ice-cold 1 M sorbitol, and then centrifuged at 1,500 g for 5 min at 4° C. and resuspended in 0.5 mL of ice-cold 1 M sorbitol (Sigma, MA). 5 μg of plasmids of interest and 5 μg of BxbI recombinase expression vector were mixed, then added to 80 μL of competent cells and incubated for 5 min in an ice-cold 0.2 cm electroporation cuvette (Bio-Rad Laboratories, CA). Pulse parameters were 1,500 V, 200Ω, and 25 μF Immediately after pulsing, 1 ml of ice-cold 1 M sorbitol was added to the cuvette, and the cuvette content was transferred to a sterile culture tube containing 1 mL 2x YPD. The culture tubes were grown overnight at 30° C. at 250 rpm. Samples were then spread on YPD plates (1% yeast extract, 2% peptone, 1 M sorbitol, 1% dextrose, and 2% agar) with 75 μg/ml zeocin (ThermoFisher, MA).

### SDS-PAGE and Western Blotting

For reducing SDS-PAGE, 30 μL of cell supernatants or purified samples were mixed with 10 μL loading dye and 4 μL 2-mercaptoethanol (ThermoFisher, MA) and heated at 90° C. for 10 min. For non-reducing SDS-PAGE, 30 μL of cell supernatants or purified samples were mixed with 10 μL loading dye and heated at 70° C. for 10 min. The samples were loaded into NuPAGE Bis-Tris pre-cast gels (ThermoFisher, MA) and run for 35 minutes at 200V in MES buffer (ThermoFisher, MA).

Gels were transferred to PVDF membranes using iBlot system (ThermoFisher, MA) according to the manufacturer's protocol. Membranes were blocked overnight using Detector Block blocking buffer (Kirkegaard & Perry Laboratories, MD) and washed three times using phosphate buffered saline with tween 20 (PBST) for 5 min. Membranes were incubated with primary antibodies overnight and then with secondary antibodies for 3 hours. The intensity of bands was analyzed using ImageJ.

Primary antibodies used in this study: anti-hGH (ab155972, Abcam, MA): 2000× dilution; anti-Interferon (ab14039, Abcam, MA): 2000× dilution; anti-G-CSF (AHC2034, ThermoFisher, MA): 2000× dilution; anti-HSA (ab84348, Abcam, MA): 2000× dilution; anti-human antibody heavy chain (MAB1302, EMD Millipore, MA): 2000× dilution; anti-human antibody light chain (ab1050, Abcam, MA): 2000× dilution.

Secondary antibodies used in this study: Rabbit anti-Mouse IgG H&L (HRP) (ab6728, Abcam, MA): 5000× dilution; Rabbit anti-Chicken IgY H&L (HRP) (ab6753, Abcam, MA): 5000× dilution; Goat anti-rabbit IgG (HRP) (7074S, Cell Signaling Technology, MA): 2000× dilution.

### LabChip Protein Expression Analysis

P. pastoris (K. phaffi) cells (pPP363, pPP364, and pJCO21) were inoculated (at OD of 0.05) in 2 mL BMGY medium in 24 deep-well plates and grown at 30° C. and 800 rpm for 48 hours. Cells were pelleted, resuspended in induction medium, and cultured at 30° C. at 800 rpm for another 48 hours. For methanol induction, cells were supplemented every 24 hours with 1% methanol. The protein titers were measured using Protein Express Assay LabChip kits (760499, PerkinElmer, MA) in LabChip GX II Touch system (PerkinElmer, MA) (FIG. 18B and FIGS. 22A-22D).

### Expression and Purification of Antibodies

P. pastoris (K. phaffi) cells (pJC110 and pJC111) were inoculated into 1 mL BMGY medium and grown at 30° C. at 250 rpm overnight. The resulting culture was inoculated at OD of 0.05 into 200 mL BMGY medium and grown at 30° C. at 250 rpm for another 48 hours. The cells were then induced in 200 mL BMMY medium with 1 μM pepstatin A (P5318-5MG, Sigma, MO) and chymostatin (C7268-5MG, Sigma, MO) and cultured at 25° C. and shaken at 250 rpm for 96 hours, and supplemented with 1% methanol and 1 μM of pepstatin A and chymostatin every 24 hours. The supernatant was dialyzed in 20 mM sodium phosphate (pH=7.0) and purified using a Protein G column (GE Healthcare, MA) according to the manufacturer's manual. The buffer of purified antibodies was then changed to phosphate-buffered saline (PBS) (ThermoFisher, MA) using PD-10 Desalting Columns (GE Healthcare, MA) (FIGS. 25A-25B).

### Activation of Human Primary T Cells and Cell Binding Assays

Human peripheral blood mononuclear cells (PBMCs) were obtained from a leukoreduction collar (Brigham and Women's hospital Crimson Core Laboratory, MA) with gradient centrifugation. Human PBMCs were activated with phytohemagglutinin (PHA) and cultured in Roswell Park Memorial Institute 1640 medium (ThermoFisher, MA), supplemented with 10% Fetal bovine serum (FBS), 10 mM HEPES, 0.1 mM non-essential amino acids, 1 mM sodium pyruvate, 100 U/mL penicillin, 100 μg/mL streptomycin, 50 μM 2-ME, and 50 IU/mL rhIL-2 (NCI, MD) for 3 days or 10 days before being used for validating anti-CTLA4 antibody and anti-PD1 antibody production. PHA-activated PMBCs were incubated with purified anti-CTLA4 antibody and/or anti-PD1 antibody at 4° C. for 25 minutes, then incubated with commercial phycoerythrin (PE)-labeled anti-human CD279 (PD-1) [329920, BioLegend, CA] or PE-labeled anti-human CD152 (CTLA4) [349906, BioLegend, CA]. Flow cytometry analysis was done by LSRII Fortessa cytometer (BD Biosciences, CA). Data analysis was done by FlowJo software (TreeStar Inc, OR) (FIG. 19E).

### Expression and Separation of Protein Mixtures

P. pastoris (K. phaffi) cells (pJC135) were inoculated into 1 mL BMGY medium and grown at 30° C. and 250 rpm overnight. The resulting culture was inoculated at OD of 0.05 into 50 mL BMGY medium and grown at 30° C. and 250 rpm for another 48 hours. The cells were then induced in 50 mL BMMY medium with 1 μM estrogen (E4389-100MG, Sigma, MO) and 1% L81 (435430-250ML, Sigma, MO) at 30° C. and 250 rpm for 48 hours, and supplemented with 1% methanol every 24 hours. The supernatant was dialyzed in 20 mM sodium phosphate (pH=7.0). 5 mL of the resulting supernatant was injected into a 1 mL Blue Sepharose Column and eluted using 5 mL elution buffer (20 mM sodium phosphate and 2000 mM sodium chloride, PH=7.0). The eluted component was then concentrated using an Amicon ultra-15 centrifugal filter (UFC901024, EMD Millipore, MA) (FIG. 20C).

HSA and hGH (A7736-1G, Sigma, MO) were separated and collected using RP-HPLC under the following conditions. Column: C4; Buffer A: 0.05% TFA; Buffer B: 0.043% TFA, 80% CAN; Gradient: 5% B/5 min-100% B/45 min; Inject amount: 50 μL; Flow rate: 0.3 ml/min; Detectors: 210 nm, 280 nm (FIG. 20C).

### Separation of hGH and HSA Using Blue Sepharose Column

100 mg hGH and 100 mg HSA were mixed and diluted in 5 mL PBS. The solution was injected into a 1 mL Blue Sepharose column. The first fraction (mainly hGH) was eluted with 5 mL low salt buffer (20 mM sodium phosphate and 100 mM sodium chloride, pH=7.0), and the second fraction (mainly HSA) was eluted with 5 mL high salt buffer (20 mM sodium phosphate and 2000 mM sodium chloride, pH=7.0) (FIG. 29). The supernatant consisting of hGH and HSA was separated as described above (FIG. 20E and FIG. 20F).

P. pastoris (K. phaffi) cells (pJC135 and pJC110) were inoculated into 1 mL BMGY medium and grown at 30° C. and 250 rpm overnight. Each of the resulting cultures was inoculated at OD of 0.05 into 200 mL BMGY medium and grown at 30° C. and 250 rpm for another 48 hours. The cells were then induced in 200 mL BMMY medium with 1% L81 (435430-250ML, Sigma, MO) at 25° C. and 250 rpm for 48 hours, and supplemented with 1% methanol and with 1 μM pepstatin A and chymostatin every 24 hours. The supernatant was dialyzed in 20 mM sodium phosphate (pH=7.0). 5 mL of the resulting supernatant was injected into a 1 mL Protein A Column (GE Healthcare, MA) and washed with 5 mL 20 mM sodium phosphate (pH=7.0) and then eluted using 2 mL elution buffer (anti-PD1 antibody) (0.1 M citric acid, pH=3.0). The flow-through was injected into a 1 mL Blue Sepharose Column. The first fraction (mainly hGH) was eluted with 5 mL low salt buffer (20 mM sodium phosphate and 100 sodium chloride, pH=7.0), and the second fraction (mainly HSA) was eluted with 5 mL high salt buffer (20 mM sodium phosphate and 2000 sodium chloride, pH=7.0) (FIG. 20G and FIG. 20H).

### Flow Cytometry

P. pastoris (K. phaffi) cells (pPP309) were inoculated at OD of 0.05 in 1 mL of BMGY and grown at 30° C. and shaken at 250 rpm for 48 hours. The resulting cultures were then cultured in induction medium with different concentration of IPTG (Gold Biotechnology, MO) for another 48 hours. 50 μL of the cultures was added to 500 μL PBS for flow cytometry analysis in a BD LSR II flow cytometer (FIG. 21B).

P. pastoris (K. phaffi) cells (pJC101) were inoculated at OD of 0.05 in 2 mL of BMGY in 24 deep-well plates and grown at 30° C. and shaken at 800 rpm for 48 hours. The resulting cultures were then cultured in induction medium consisting of methanol, estrogen, or IPTG for another 48 hours. 50 μL of the cultures was added to 500 μL PBS for flow cytometry analysis in a BD LSR II flow cytometer (FIG. 21C, FIG. 21D).

### REFERENCES FOR EXAMPLE 2

1. Gray, A. & Manasse, H. R., Jr. Shortages of medicines: a complex global challenge. Bull World Health Organ 90, 158-158A (2012).
2. Ventola, C. L. The drug shortage crisis in the United States: causes, impact, and management strategies. P T 36, 740-757 (2011).
3. Cefalu, W. T., Smith, S. R., Blonde, L. & Fonseca, V. The hurricane Katrina aftermath and its impact on diabetes care—Observations from “ground zero”: lessons in disaster preparedness of people with diabetes. Diabetes Care 29, 158-160 (2006).
4. Adamo, A. et al. On-demand continuous-flow production of pharmaceuticals in a compact, reconfigurable system. Science 352, 61-67, doi:10.1126/science.aaf1337 (2016).
5. Pardee, K. et al. Portable, On-Demand Biomolecular Manufacturing. Cell 167, 248-259 (2016).
6. Perez-Pinera, P. et al. Synthetic biology and microbioreactor platforms for programmable production of biologics at the point-of-care. Nat Commun 7, 12211 (2016).
7. Dove, A. Uncorking the biomanufacturing bottleneck. Nat Biotechnol 20, 777-779 (2002).
8. Gottschalk, U., Brorson, K. & Shukla, A. A. The need for innovation in biomanufacturing. Nat Biotechnol 30, 489-492 (2012).
9. Flemming, A. Anticancer drugs: Finding the perfect combination. Nat Rev Drug Discov 14, 13 (2015).
10. Zhang, L. et al. Quantifying residual HIV-1 replication in patients receiving combination antiretroviral therapy. N Engl J Med 340, 1605-1613 (1999).
11. Skibinski, D. A., Baudner, B. C., Singh, M. & O'Hagan, D. T. Combination vaccines. J Glob Infect Dis 3, 63-72 (2011).
12. Frandsen, T. P. et al. Consistent Manufacturing and Quality Control of a Highly Complex Recombinant Polyclonal Antibody Product for Human Therapeutic Use. Biotechnology and Bioengineering 108, 2171-2181 (2011).
13. Rasmussen, S. K., Naested, H., Muller, C., Tolstrup, A. B. & Frandsen, T. P. Recombinant antibody mixtures: Production strategies and cost considerations. Archives of Biochemistry and Biophysics 526, 139-145 (2012).
14. Dienstmann, R. et al. Safety and Activity of the First-in-Class Sym004 Anti-EGFR Antibody Mixture in Patients with Refractory Colorectal Cancer. Cancer Discov 5, 598-609 (2015).
15. Raju, T. S. & Strohl, W. R. Potential therapeutic roles for antibody mixtures. Expert Opin Biol Th 13, 1347-1352 (2013).
16. Qiu, X. et al. Reversion of advanced Ebola virus disease in nonhuman primates with ZMapp. Nature 514, 47-53 (2014).
17. Byrd, J. C. et al. Phase 1/2 study of lumiliximab combined with fludarabine, cyclophosphamide, and rituximab in patients with relapsed or refractory chronic lymphocytic leukemia. Blood 115, 489-495 (2010).
18. Boutros, C. et al. Safety profiles of anti-CTLA-4 and anti-PD-1 antibodies alone and in combination. Nat Rev Clin Oncol 13, 473-486 (2016).
19. Sully, E. K. et al. A tripartite cocktail of chimeric monoclonal antibodies passively protects mice against ricin, staphylococcal enterotoxin B and Clostridium perfringens epsilon toxin. Toxicon 92, 36-41 (2014).
20. Muller, T. et al. Development of a mouse monoclonal antibody cocktail for post-exposure rabies prophylaxis in humans. PLoS Negl Trop Dis 3, e542 (2009).
21. Valsecchi, M. E. Combined Nivolumab and Ipilimumab or Monotherapy in Untreated Melanoma. New Engl J Med 373, 1270-1270 (2015).
22. Jayapal, K. R., Wlaschin, K. F., Hu, W. S. & Yap, M. G. S. Recombinant protein therapeutics from CHO cells—20 years and counting. Chem Eng Prog 103, 40-47 (2007).
23. Vogl, T., Hartner, F. S. & Glieder, A. New opportunities by synthetic biology for biopharmaceutical production in Pichia pastoris. Curr Opin Biotechnol 24, 1094-1101 (2013).
24. Ahmad, M., Hirz, M., Pichler, H. & Schwab, H. Protein expression in Pichia pastoris: recent achievements and perspectives for heterologous protein production. Appl Microbiol Biotechnol 98, 5301-5317 (2014).
25. Shah, K. A. et al. Automated pipeline for rapid production and screening of HIV-specific monoclonal antibodies using pichia pastoris. Biotechnol Bioeng 112, 2624-2629 (2015).
26. Choi, B. K. et al. Use of combinatorial genetic libraries to humanize N-linked glycosylation in the yeast Pichia pastoris. Proc Natl Acad Sci USA 100, 5022-5027 (2003).
27. Li, H. et al. Optimization of humanized IgGs in glycoengineered Pichia pastoris. Nat Biotechnol 24, 210-215 (2006).
28. Rasmussen, S. K. et al. Recombinant antibody mixtures; optimization of cell line generation and single-batch manufacturing processes. BMC Proc 5 Suppl 8, 02 (2011).
29. Zhang, H., Pereira, B., Li, Z. & Stephanopoulos, G. Engineering Escherichia coli coculture systems for the production of biochemical products. Proc Natl Acad Sci USA 112, 8266-8271 (2015).
30. Frokjaer, S. & Otzen, D. E. Protein drug stability: a formulation challenge. Nat Rev Drug Discov 4, 298-306 (2005).
31. Tarelli, E. et al. Recombinant human albumin as a stabilizer for biological materials and for the preparation of international reference reagents. Biologicals 26, 331-346, doi:10.1006/biol.1998.0163 (1998).
32. Chuang, V. T. G., Kragh-Hansen, U. & Otagiri, M. Pharmaceutical strategies utilizing recombinant human serum albumin. Pharmaceut Res 19, 569-577 (2002).
33. Jeyachandran, Y. L., Mielczarski, E., Rai, B. & Mielczarski, J. A. Quantitative and qualitative evaluation of adsorption/desorption of bovine serum albumin on hydrophilic and hydrophobic surfaces. Langmuir 25, 11614-11620 (2009).
34. Lei, J. et al. Expression, purification and characterization of recombinant human interleukin-2-serum albumin (rhIL-2-HSA) fusion protein in Pichia pastoris. Protein Expr Purif 84, 154-160 (2012).
35. Kobayashi, K. et al. High-level expression of recombinant human serum albumin from the methylotrophic yeast Pichia pastoris with minimal protease production and activation. J Biosci Bioeng 89, 55-61 (2000).
36. Huang, Y. S. et al. Development and characterization of a novel fusion protein of a mutated granulocyte colony-stimulating factor and human serum albumin in Pichia pastoris. PLoS One 9, e115840 (2014).
37. Rayner, J. C. & Munro, S. Identification of the MNN2 and MNN5 mannosyltransferases required for forming and extending the mannose branches of the outer chain mannans of Saccharomyces cerevisiae. J Biol Chem 273, 26836-26843 (1998).
38. Kapust, R. B. et al. Tobacco etch virus protease: mechanism of autolysis and rational design of stable mutants with wild-type catalytic proficiency. Protein Engineering 14, 993-1000 (2001).
39. Kapust, R. B., Tozser, J., Copeland, T. D. & Waugh, D. S. The P1′ specificity of tobacco etch virus protease. Biochem Biophys Res Commun 294, 949-955 (2002).
40. Wehr, M. C. et al. Monitoring regulated protein-protein interactions using split TEV. Nat Methods 3, 985-993 (2006).
41. Szymczak, A. L. et al. Correction of multi-gene deficiency in vivo using a single ‘self-cleaving’ 2A peptide-based retroviral vector. Nat Biotechnol 22 (2004).
42. Pardoll, D. M. The blockade of immune checkpoints in cancer immunotherapy. Nat Rev Cancer 12, 252-264 (2012).
43. Legat, A., Speiser, D. E., Pircher, H., Zehn, D. & Fuertes Marraco, S. A. Inhibitory Receptor Expression Depends More Dominantly on Differentiation and Activation than “Exhaustion” of Human CD8 T Cells. Front Immunol 4, 455 (2013).
44. Steel, L. F. et al. Efficient and specific removal of albumin from human serum samples. Mol Cell Proteomics 2, 262-270 (2003).
45. Gardner, T. S., Cantor, C. R. & Collins, J. J. Construction of a genetic toggle switch in Escherichia coli. Nature 403, 339-342 (2000).
46. Deans, T. L., Cantor, C. R. & Collins, J. J. A tunable genetic switch based on RNAi and repressor proteins for regulating gene expression in mammalian cells. Cell 130, 363-372 (2007).
47. Card, J. W. et al. Proof of concept for a banding scheme to support risk assessments related to multi-product biologics manufacturing. Regul Toxicol Pharmacol 73, 595-606 (2015).
48. Carver, M. Applying a Health- and Science-based Risk Assessment for Multi-Product Biologics manufacturing (white paper). (2013).
49. Warikoo, V. et al. Integrated continuous production of recombinant therapeutic proteins. Biotechnol Bioeng 109, 3018-3029 (2012).

### Example 3: Production of Functional Anti-Ebola Antibodies in Komagataella phaffi (Pichia pastoris)

The 2013-2016 Ebola outbreak in western Africa exposed the limited treatment options for patients infected with Ebola virus. Since the outbreak, substantial resources have been spent to expand the range and increase the availability of treatment options. These efforts can be divided into two main approaches: 1) development of Ebola vaccines for pre- and post-infection application^{1,2}, and 2) development of therapeutics for infected people, for whom prevention was not available or failed. For the latter, a number of different approaches are being taken, including siRNAs³, antisense oligonucleotides^{4,5}, a nucleoside analog⁶, and a neutralizing cocktail of three monoclonal antibodies (mAbs) referred to as ZMapp^{7,8}. ZMapp (specifically ZMapp1) has been shown to rescue 100% of Rhesus macaques when administered up to 5 days post infection⁸. ZMapp mAbs bind to the Ebola glycoprotein (GP)⁹, likely preventing GP-mediated entry of the virus into human cells.

The component antibodies of ZMapp are currently produced in the plant Nicotiana benthamiana⁸. During the 2013-2016 outbreak, the limited supplies of ZMapp were quickly exhausted. In addition to the need to quickly increase supply of antibodies, evolution in the Ebola virus genome, particularly of the Ebola glycoporotein if ZMapp or other anti-GP mAbs are used, will likely necessitate the rapid development of new versions of these cocktails to maintain treatment effectiveness¹⁰. A recent analysis of neutralizing antibodies from survivors of the 2007 Uganda Bundibugyo ebolavirus (BDBV) outbreak isolated 90 mAbs, 57 of which cross-reacted with Ebola virus (EBOV), a number of them being considered potent with low nanomolar affinities¹¹. A collection of potent neutralizing mAbs were also isolated from a single survivor of the 2013-2016 Ebola (EBOV Zaire) outbreak¹². This diversity and potency suggests that an infected population is a rich source of new neutralizing mAbs with which to combat the outbreak. Thus, efficient strategies that can take advantage of this diversity and rapidly produce recombinant neutralizing antibodies are needed. Furthermore, ZMapp is a cocktail of chimeric antibodies that uses murine variable regions which cause immunogenicity when administered to humans. Thus, it may be beneficial to derive variable chains from human survivors to create fully human mAbs with reduced immunogenicity.

Additionally, to meet the need for new therapies for emerging disease and outbreaks, alternative hosts for producing mAbs that are amenable to rapid engineering and scalable production are being sought. Chinese Hamster Ovary (CHO) cells are a frequently used source for mAb production due to their ability to produce human-like post-translational modifications. However, certain characteristics of CHO cell production, such as the risk of viral contamination¹³ and slow growth rate, make CHO cells less than optimal in pathogen outbreaks where the speed of the development cycle is critical to treating as many patients as quickly as possible. An alternative, the yeast K. phaffi (Pichia pastoris), is a well-developed host for the production of biopharmaceuticals, offering potentially reduced development times and high-product yields^{14,17}. Glycoengineered strains of K. phaffi, with humanized N-linked glycosylation profiles, minimize potential issues of immunogenicity and low affinity that can be caused by yeast N-linked glycosylation^{18,21}. There are now many K. phaffi-derived products on the market, such as Kalbitor (approved in the US), a kallikrein inhibitor, and Insugen, a recombinant human insulin (approved in >40 countries). Currently, two different K. phaffi-derived therapeutic antibody fragments are in clinical trials; Nanobody® ALX0061 (Phase IIb) and Nanobody® ALX00171 (Phase IIa), which are being studied for treating rheumatoid arthritis and respiratory syncytial virus infections, respectivelyl³. Furthermore, when derived from glycoengineered K. phaffi, trastuzumab, a full length anti-cancer mAb, showed comparable pharmacokinetics and tumor inhibitory efficacy to CHO-cell-derived trastuzumab²².

One limitation of K. phaffi has been that integration by homologous recombination of linearized plasmids has been the only option for strain construction, which is not optimal for the initial testing of a library of candidate molecules (e.g., mAbs) where rapid strain construction is desirable. Homologous recombination requires the two steps of linearization and subsequent cleanup of the construct, and may require re-design of the construct to ensure a suitable unique restriction site is available for linearization. Described herein is a K. phaffi strain with a set of integrated recombinase “landing pads” that enable reliable targeted genomic integration across a range of construct sizes without the need for linearization and cleanup. The removal of these two steps provides significant advantage in the speed and cost of constructing a large-scale mAb screening library. Here, this approach is adopted to create a landing pad strain variant of Pichia GLYCOSWITCH® (RCT, AZ, USA), a commercial strain engineered for high expression of proteins with human-like glycosylation, to produce the ZMapp cocktail. The Pichia GLYCOSWITCH® SuperMan5(His+) variant produces a low-mannose Man5GlcNAc2 glycoform as the major composition, in contrast to the Man8GlcNAc2 and higher order hyper-glycosylated structures that otherwise occur²¹. High-mannose glycoforms have been shown to increase the clearance rate of therapeutic IgG antibodies in humans,²⁴ therefore production of antibodies without the high-mannose glycoforms may provide therapeutic benefits. As described herein, the K. phaffi-derived antibodies obtained using the engineered K. phaffi strains were functional, opening up the possibility that glycoengineered yeast can be a host for the rapid production of therapeutic antibodies, such as ZMapp.

Engineering of (K. phaffi) Pichia pastoris for Production of ZMapp Antibodies

Inefficient integration and genome engineering is a disadvantage of K. phaffi, and a potential bottleneck in creating therapeutic antibodies, especially when numerous strains, each producing a mAb with a new variable chain, are needed to address evolving pathogens. In prior work²³, we engineered a K. phaffi strain with a set of integrated recombinase “landing-pads” to achieve quick and reliable integration across a range of construct sizes up to 13.6 kb. We have adapted this approach here by creating a landing-pad version of the Pichia GLYCOSWITCH® strain to accelerate the genetic engineering of these strains for the production of mAbs. Our strain contained recombination sites for the Serine Recombinases BxbI, TP901-1 and R4, integrated into the genome at the Trp2 locus. Integration into the landing pads is achieved by co-transformation of the plasmid to be integrated with a plasmid constitutively expressing the recombinase (FIG. 22B). An advantage of yeast over CHO cells as a production platform is their relative ease of genetic manipulation^{13,15}, and the use of genomic landing-pads simplifies this further.

We performed a direct comparison of the transformation efficiencies achieved by recombinase-mediated integration versus integration by homologous recombination of linearized plasmid DNA (see Materials and Methods for details. Integration by recombinase-mediated integration and integration by homologous recombination of linearized plasmid DNA produced 233.3±107.6 and 9205.6±2298.2 (in both cases mean±s.d, n=3) transformants per μg DNA, respectively. This result allowed us to distinguish the different scenarios in which recombinase-mediated integration and integration by homologous recombination of linearized plasmid DNA are most suited. Recombinase-mediated integration is useful for the initial transformation of a large library of variants where a relatively small amount of product is required to perform functional assays. Integration is limited to single copy, but fewer steps are required compared with integration by homologous recombination of linearized plasmid DNA. This feature is particularly important when performing large numbers of independent transformations and/or automating the process on robotic platforms. Integration by homologous recombination of linearized plasmid DNA is suited to the subsequent refinement and optimization phase, where a subset of the most promising product candidates is assessed for scaling up in product yield. Integration by homologous recombination of linearized plasmid DNA not only permits multi-copy integration but also produces more transformants per μg of DNA (˜40-fold more in our data), giving a larger pool from which to screen for high-producers.

The three monoclonal ZMapp antibodies 2G4, 4G7 and 13C6 were produced each in a different Pichia GLYCOSWITCH® landing pad clone at laboratory scale. Antibodies 4G7 and 13C6 were also produced from strains created using linearization-based integration of the plasmid. According to ELISA and Surface Plasmon Resonance (SPR) analysis (Table 9), we were able to obtain yields in the 1-10 mg·L⁻¹ range for all three mAbs. These yields were sufficient for a first set of expression clones given that 1,000 mg·L⁻¹ has been reported after extensive strain development for glycoengineered K. phaffi¹⁴. In addition, these yields are on the order of some pilot studies for single-chain variable fragment (ScFv) production from K. phaffi²⁵′²⁶ and are not far below early stage yields for neutralizing HIV mAbs from non-glycoengineered K. phaffi²⁷. For both 4G7 and 13C6, we were able to achieve higher yields from clones generated using integration by homologous recombination of linearized plasmid DNA than clones generated using recombinase-mediated integration (data not shown), likely owing to multi-copy integrations.

Interestingly, concentrations determined by ELISA were lower than those based on SPR. We speculate that this difference could be due to the fact that the Protein A ligand used on the SPR chips can bind to mAb fragments present in the bulk supernatant before chromatographic purification as well as the fully assembled antibody, whereas ELISA should only assay the fully assembled antibody. Additionally, we found that adding Casamino acids to the cultivation medium increased the mAb concentration at the end of the fermentation by a factor of ˜1.9±0.3 (mean±s.d, n=4), yielding a total of ˜25 mg·L⁻¹, which is in agreement with previous reports using this substance²⁸. In particular, the concentration achieved for 2G4 was remarkably high, given the fact that the corresponding Pichia clone only harbored a single-copy integration.

TABLE 9
mAb concentration in fermentation supernatant determined by SPR and ELISA. 2G4
was produced from a strain generated by recombinase-mediated integration, while
reported values from 13C6 and 4G7 were from strains generated by integration by
homologous recombination of linearized plasmid DNA. Values are the mean ± s.d.
Values in brackets are the Space-Time Yield (STY), given as mean ± s.d and
measured in units of mg/L * h.

| | Concentration [mg · L⁻¹] | | | |
|---|---|---|---|---|
| | Basal salt medium | | Supplemented with Casamino acids | |
| Antibody | SPR | ELISA | SPR | ELISA |
| 2G4 | 10.93 ± 0.81 | 9.93 ± 0.50 | 26.53 ± 1.11 | 18.98 ± 2.05 |
| | (0.077 ± 0.006) | (0.070 ± 0.004) | (0.196 ± 0.008) | (0.141 ± 0.015) |
| 13C6 | 4.16 ± 0.09 | 0.68 ± 0.04 | 6.30 ± 0.13 | 1.10 ± 0.07 |
| | (0.028 ± 0.001) | (0.005 ± 0.0003) | (0.051 ± 0.001) | (0.009 ± 0.001) |
| 4G7 | 3.74 ± 0.42 | 0.44 ± 0.02 | n.a. | n.a. |
| | (0.027 ± 0.003) | (0.003 ± 0.0001) | | |

We tested two affinity ligands for the initial capture of the mAbs from clarified fermentation supernatant and found that the highest purities for mAbs 2G4 and 4G7 were achieved with Protein A, whereas Protein G was optimal for mAb 13C6 (data not shown). After a subsequent cation exchange chromatography (CEX), the mAb purity was assessed based on densitometric analysis of Coomassie-stained LDS-PAA-gels (FIG. 22C). The heavy chain (HC) and light chain (LC) ran at the expected molecular masses, as indicated by the positive control (FIG. 22C, lane 4).

### Immunofluorescence Assay

FIGS. 23A-23C show immunofluorescence assays (IFAs) for the three mAbs, all produced from strains with mAbs integrated by recombinase-mediated integration. For each of our three mAbs, there was clearly binding of the mAb to cells transfected with pCAGGS-ZEBOV GP1,2³⁰, but not to untransfected cells. pCAGGS-ZEBOV GP1,2 expresses the Zaire Ebola virus (Mayinga strain) Glycoprotein, which becomes membrane associated³⁰. Some untransfected cells treated with the 2G4 mAb showed very weak background GFP fluorescence, which could be due to low-affinity non-specific binding to the membrane. These results agreed qualitatively with the IFAs performed on ZMapp mAbs in Qiu et al³⁰.

The 2013-2016 Ebola outbreak exposed both the paucity of available anti-Ebola treatment options and our inability to provide them rapidly and at scale. ZMapp, a cocktail of three anti-Ebola neutralizing mAbs, is a promising treatment that has demonstrated efficacy in non-human primates, and is currently in human clinical trials. Production of anti-Ebola neutralizing mAbs has been demonstrated in CHO cells and ZMapp is currently produced in the plant N. benthamiana. The yeast K. phaffi is an alternative production platform for therapeutic biopharmaceuticals, including mAbs, and has desirable characteristics such as ease of scaling and short development times. It is therefore an excellent candidate for production of anti-Ebola mAbs, including ZMapp.

Here, we engineered a landing pad system into Pichia GLYCOSWITCH® and used the resulting strain to produce the constituent antibodies of the anti-Ebola ZMapp cocktail. Immunofluorescence assays gave comparable results to previous studies conducted on ZMapp antibodies derived from mice³⁰, and demonstrated that the antibodies bind to the GP component of Ebola in vitro. Future studies should examine the efficacy of K. phaffi-derived ZMapp antibodies in animal models, both in mice and non-human primates. Furthermore, optimizing the production process to increase product yield, product quality (in terms of solubility, aggregation, degradation, and so forth), glycoform profile, and other parameters was not the priority of this work but is vital for producing a therapeutic product.

With this platform, we propose landing-pad glycoengineered K. phaffi as the enabling component of a rapid development cycle (FIG. 24) in which an at-risk population is 1) monitored for infection by a specific pathogen, 2) neutralizing antibodies are isolated from those infected and the variable regions sequenced^{11,31}, 3) DNA constructs of chimeric mAbs using the new variable regions and common constant regions are synthesized, 4) landing-pad glycoengineered K. phaffi strains are rapidly generated using recombinase-mediated integration to express the new candidate mAbs, 5) promising candidate mAbs are produced and tested for therapeutic efficacy, 6) production of effective mAbs is scaled-up, using integration by homologous recombination of linearized plasmid DNA to screen for high-producing, likely multi-copy clones, either as centralized production with distribution or local small-scale production using microbioreactors²³, and 7) infected people are treated as soon as possible using the mAbs. Although the ability of integration by homologous recombination of linearized plasmid DNA to generate multi-copy strains should on average allow the generation of higher-producing strains, our results show that single-copy strains generated by recombinase-mediated integration can also generate comparable yields.

In addition to Ebola, our strategy could be applied to other pathogen outbreaks where the speed of the outbreak, the potential for loss of life, and continuous evolution of the pathogen means that traditional and slow approaches for prototyping and manufacturing neutralizing antibodies are not optimal. This strategy could potentially be enabled by the FDA Fast Track program, under which the existing ZMapp cocktail is being evaluated³². The recent outbreak of Zika virus in South America is another prime example of where such a development cycle could be applied, with the first anti-Zika neutralizing antibodies recently being discovered³¹. There has been a drive towards real-time monitoring of pathogenic viruses in outbreaks and at-risk populations³³ and thus, this platform may enable rapid response to these emerging and evolving pathogens with neutralizing therapeutics.

### Materials and Methods

### Construct Design and mAb Sequences

The DNA sequences for expressing mAbs 2G4, 4G7 and 13C6, were constructed on separate plasmids (pOP459, pOP461 and pOP462 respectively). Constructs (FIG. 1A) were synthesized as multiple geneBlocks (IDT, IA) and assembled by Gibson assembly³⁴. In each case, the methanol-induced pAOX1 promoter was used to express the mAb. Each mAb was constructed as a single open reading frame, with the following structure, AF-HC-T2A-AF-LC, where AF is the alpha factor secretion tag, HC and LC are the heavy and light chain respectively and T2A is a sequence that causes a ribosomal “skip”²⁹, resulting in the alpha-factor-tagged heavy and light chains being secreted as separate polypeptides. A GSG linker preceded the T2A sequence to ensure cleavage is maximally efficient (˜100% typically seen)²⁹. 2A sequences have been used previously in this way to link heavy and light chains for mAb production^{35,36}. The AOX1 terminator was used in all cases. The constant region of the heavy chain was IgG-1, adapted from Uniprot P01857 to ensure no duplication of residues between the end of the variable region and the start of the constant region. The constant region of the light chain was the Kappa variant and taken from Uniprot P01834. Sequences for the variable chains (heavy and light) of 2G4 and 4G7 were obtained from U.S. Pat. No. 8,513,391 B2, and of 13C6 from US patent application 2004/0053865 A1.

### Strain Construction

To facilitate subsequent integration steps, PP74²³, a plasmid containing a set of landing pads for the recombinases BxbI, TP901-1 and R4 was first linearized and then integrated into Pichia GLYCOSWITCH® SuperMan5(His+) (RCT, AZ, USA) at the Trp2 locus (chromosome II: 286540-28607). Integration of the landing pad was selected for by G418 resistance. This landing pad strain was then used to integrate plasmids containing expression constructs for 2G4, 4G7 and 13C6 into the BxbI landing pad to generate K. phaffi strains PP21A, PP22A and PP23A, respectively. Plasmids were integrated using a standard K. phaffi electroporation protocol³⁷. Five μg of the helper plasmid containing constitutively expressed BxbI recombinase was co-transformed along with the plasmid to be integrated. All clones were selected in Zeocin and integrations were verified by colony PCR using Robust 2G Polymerase (Kapa Biosystems). For the construction of strains harbouring 4G7 and 13C6 created by linearization-integration, the protocol used was identical to that used in the transformation efficiency comparison, and clones were selected on 500 μg/ml Zeocin plates to select for multi-copy clones (integration copy number was not assessed). All constructs will be available on Addgene.

### Transformation Efficiency Comparison

Pichia GLYCOSWITCH® integrated with PP74 was used for integration of pOP462 (containing mAb 13C6) either by recombinase-mediated integration at the BxbI landing pad or by homologous recombination of linearized plasmid DNA. For linearization, an extended (<16 hours) digestion of pOP462 was performed with DraI. The sample was then cleaned-up using ethanol precipitation, and a sample of the cleaned-up linear DNA was analyzed using gel electrophoresis to ensure linearization was complete. Cell growth and transformation was performed largely as previously described²³. 5 ml of 2xYPD was inoculated directly from frozen stock, and the cells grown overnight at 30° C. with shaking. 100 ml of 2xYPD was then inoculated with 50 μl of the overnight culture and the cells were grown overnight to O.D˜1.6. Cells were then centrifuged at 1,500 g for 5 min at 4° C. and resuspended with 40 ml of ice-cold sterile water, centrifuged at 1,500 g for 5 min at 4° C. and resuspended with 20 ml of ice-cold sterile water, centrifuged at 1,500 g for 5 min at 4° C. and resuspended in 20 ml of ice-cold 1.0 M sorbitol, and centrifuged at 1,500 g for 5 min at 4° C. and resuspended in 1 ml of ice-cold 1.0 M sorbitol. 80 μl of cells were then added to each ice-cold 0.2 cm electroporation cuvette and the required DNA (either linearized pOP462, or uncut pOP462 plus the BxbI expressing helper plasmid) added and mixed. To ensure the validity of comparison, the concentrations of the linearized and uncut pOP462 were adjusted so that the volume of linearized pOP462 and the combined volume of uncut pOP462 and the BxbI expressing helper plasmid were approximately equal. 5 μg of linearized pOP462 and 5 μg uncut pOP462 plus 5 μg of BxbI expressing helper plasmid were used. Pulse parameters were 1,500 V, 200Ω and 25 μF. Immediately after pulsing, 1 ml of ice-cold 1.0 M sorbitol was added to the cuvette and the cuvette content was transferred to a sterile culture tube containing 2xYPD. The culture tubes were incubated for 2 h at 30° C. with shaking and 75 μl of the culture was spread on Yeast Extract Peptone Dextrose Sorbitol (YPDS) plates (1% [w/v] yeast extract, 2% [w/v] peptone, 1.0 sorbitol, 1% [w/v] dextrose and 2% [w/v] agar) with the appropriate selection antibiotic (Zeocin 100 mg·ml). Plates were incubated for 2 days at 30° C. before colony counts were performed.

K. phaffi Cultivation

Pre-cultures were carried out in 500 mL baffled flasks with 150 mL YSG (10 g·L⁻¹ yeast extract, 20 g·L⁻¹ soy peptone and 20 g·L⁻¹ glycerol). All pre-cultures were inoculated from a single colony grown on YPD-Zeocin (100 μg·mL⁻¹ Zeocin) plates and were incubated for 24 h at 28° C. on a rotary shaker at 300 rpm. Bioreactors (either a Bio Bench 7 (Applikon, Delft, Netherlands) or a Bio Pilot 40 (Applikon, Delft, Netherlands)) were subsequently inoculated with 10% [v/v] of the pre-culture and incubated for ˜30 h at 28° C. using basal salts medium (26.7 mL·L⁻¹ 85% phosphoric acid, 0.93 g·L⁻¹ calcium sulphate, 18.2 g L⁻¹ potassium sulphate, 14.9 g·L⁻¹ magnesium sulphate×7H₂O, 4.13 g·L⁻¹ potassium hydroxide, 40.0 g·L⁻¹ glycerol and 0.435% [v/v] PTM1) supplemented with 30 g·L⁻¹ glycerol and PTM1 trace elements solution A. The pH was maintained at pH 6.0 using 250 g·L⁻¹ ammonia while the dissolved oxygen tension (DOT) was maintained at 30% by varying the stirrer speed in the 350-1,000 rpm range. The aeration rate was constant at 1 vvm with 1 barg head pressure and struktol J673 (Schill & Seilacher GmbH, Hamburg, Germany) was used as an antifoaming agent. The induction of transgene expression was triggered by a limiting feed with pure methanol and lasted for ˜70 hours during which the temperature was reduced to 24° C. Optionally, the cultivation medium was supplemented with 1 g·L⁻¹ Casamino acids during the batch phase and addition of 1 g·L⁻¹ Casamino acids every 20 hours during the induction phase. OD600, cell wet weight and cell dry weight were monitored during the cultivation.

### Quantification Via SPR

A Sierra SPR4 (Sierra Sensors, Hamburg, Germany) was used for mAb quantification in samples using a Protein A labelled high capacity amine chip (Sierra Sensors, Hamburg, Germany) as described before³⁸. Samples were diluted 1:20 in HBS-EP (10 mM HEPES, 3 mM EDTA, 150 mM sodium chloride, 0.05% [v/v] Tween-20, pH 7.4). The anti-HIV mAb 2G12 (Fraunhofer IME, Germany) was used as a quantification standard.

### Quantification Via ELISA

Flat bottom, high-binding 96-well microtiter plates (Greiner Bio-One, Kremsmtinster, Austria) were coated with 100 μL·well⁻¹1:2000 diluted (in PBS) goat-α-human IgG Fc-specific antibody (Sigma-Aldrich, Seelze, Germany) over night at 4° C. Subsequently to every incubation step, the plates were washed 3 times with H₂O and once with PBST (137 mM NaCl, 2.7 mM KCl, 10.1 mM Na₂HPO₄, 1.7 mM KH₂PO₄ and 0.05% [v/v] Tween-20). Blocking was conducted with 250 μL·well⁻¹ 5% [w/v] milk powder solution in PBST, for 1 h. A two-fold serial dilution (in PBS) was prepared from 3 standards (2G12) with an initial concentration of 500 ng·mL⁻¹, selected supernatants from the cultivations and from a control prior induction. AP-labeled polyclonal goat-α-human kappa-chain specific antibody (Sigma-Aldrich, Seelze, Germany), diluted 1:5000 in PBS, was used for detection via the alkaline phosphatase color reaction. PNPP (Sigma-Aldrich, Seelze, Germany) was used as substrate for the alkaline phosphatase at a concentration of 1 mg·mL⁻¹ in alkaline phosphatase buffer (100 mM Tris-HCl, 100 mM sodium chloride, 5 mM Magnesium chloride, pH 9.6).

### Antibody Purification

Bulk fermentation broth was centrifuged for 20 min at 9,000×g and 4° C. Then, the pH of the supernatant was adjusted to 7.0 (mAb 2G4 and 4G7) or pH 6.5 (mAb 13C6) with ˜10 mL·L⁻¹ of 250 g·L⁻¹ ammonia and 0.2 μm filtered using a Sartopore 2 150 filter (Sartorius A G, Gottingen, Germany) For initial capture of mAbs 2G4 and 4G7, Protein A columns were equilibrated (50 mM NaH₂PO₄, 50 mM NaCl, pH 7.0) for 5 column volumes. After sample loading, a 5 column volume wash (25 mM Tris, 10% isopropanol, 1 M urea, pH 9.0)³⁹ preceded mAb elution (0.2 M acetic acid, 150 mM NaCl, pH 2.8). Antibody 13C6 was captured on Protein G after equilibration (25 mM Tris, 100 mM NaCl, pH 7.4) followed by a 10 column volume wash in the same buffer and subsequently eluted (0.1 M glycine, pH 2.7). All elution fractions were immediately neutralized (1M Tris-HCl, pH 9.0), diluted to a conductivity <7.5 mS cm⁻¹ if required and subjected to a isocratic cation exchange chromatography (flow-through mode; 50 mM citric acid) with individual pHs (5.5 for 2G4; 5.8 for 13C6 and 5.2 for 4G7).

### Immunofluorescence Assay

The immunofluorescence assay (IFA) was performed as detailed in Qiu et al³⁰. Briefly, in this assay cells are transfected with a plasmid that expresses the Ebola glycoprotein on the cell surface. Candidate antibodies are bound on the fixed cells to form the primary complex, to which a FITC-conjugated anti-human Ab secondary antibody is then bound on to the candidate antibodies. Detection of the secondary antibody at the cell membrane by excitation of FITC denotes a positive result. Two million HEK293T cells were trypsinized and re-suspended in 500 μL culture medium (DMEM with 10% (v/v) FBS and 1% (v/v) P/S) and then mixed thoroughly with a pre-mixed solution of 500 ng of Glycoprotein (GP)-antigen-expressing plasmid pCAGGS-ZEBOV GP1,2³⁰ (GP sequence from the GP strain Mayinga, GenBank accession no. AF272001), 12 μL FuGene HD plus transfection reagent, and 100 μL Opti-MEM. After incubation at 37° C. for 48 hours, cells were fixed with 4% paraformaldehyde in PBS, and blocked with 10% (v/v) FBS in PBS at room temperature (RT) for 2 hours. mAbs were diluted in blocking buffer, added to the cells, and incubated at RT for 1 hour. Cells were then washed 3 times with 0.1% Tween-20 in PBS. A 1:200 dilution of Alexa-Fluor488 conjugated goat anti-human IgG (Invitrogen) was added to each well and incubated at RT for 1 hour, before images were taken on a Nikon Eclipse Ti microscope at 40× magnification. Nuclei were stained with 4′,6-diamidino-2-phenylindole (DAPI) (D1306, Life Technologies; 1:1000 dilution in PBS) at RT for 5 min

### LDS-PAGE

Precast polyacrylamide 4-12% [w/v] Bis-Tris gels (Life technologies, Carlsbad, USA) were used according to the instructions provided by the manufacturer: Either 10 μL sample or 5 μL PageRuler pre-stained protein ladder (Life technologies, Carlsbad, USA) were then loaded per lane. Subsequently, gels were stained using Simply Blue Safe Stain solution (Life Technologies, Carlsbad, USA) according to the manufacturer's protocol. Gels were scanned at 600 dpi using a Canon scan 5600 scanner (Canon, Krefeld, Germany) and the software Adobe Photoshop Elements 4.0 (Photoshop, California, USA).

### REFERENCES FOR EXAMPLE 3

1. Feldmann, H. et al. Effective post-exposure treatment of Ebola infection. PLoS Pathog. 3, e2 (2007).
2. Geisbert, T. W. et al. Recombinant vesicular stomatitis virus vector mediates postexposure protection against Sudan Ebola hemorrhagic fever in nonhuman primates. J. Virol. 82, 5664-5668 (2008).
3. Thi, E. P. et al. Lipid nanoparticle siRNA treatment of Ebola-virus-Makona-infected nonhuman primates. Nature 521, 362-365 (2015).
4. Warren, T. K. et al. Advanced antisense therapies for postexposure protection against lethal filovirus infections. Nat. Med. 16, 991-994 (2010).
5. Warren, T. K. et al. A Single Phosphorodiamidate Morpholino Oligomer Targeting VP24 Protects Rhesus Monkeys against Lethal Ebola Virus Infection. M Bio 6, e02344-14 (2015).
6. Warren, T. K. et al. Protection against filovirus diseases by a novel broad-spectrum nucleoside analogue BCX4430. Nature 508, 402-405 (2014).
7. Olinger, G. G. et al. Delayed treatment of Ebola virus infection with plant-derived monoclonal antibodies provides protection in rhesus macaques. Proc. Natl. Acad. Sci. 109, 18030-18035 (2012).
8. Qiu, X. et al. Reversion of advanced Ebola virus disease in nonhuman primates with ZMapp. Nature (2014).
9. Murin, C. D. et al. Structures of protective antibodies reveal sites of vulnerability on Ebola virus. Proc. Natl. Acad. Sci. 111, 17182-17187 (2014).
10. Carroll, M. W. et al. Temporal and spatial analysis of the 2014-2015 Ebola virus outbreak in West Africa. Nature 524, 97-101 (2015).
11. Flyak, A. I. et al. Cross-Reactive and Potent Neutralizing Antibody Responses in Human Survivors of Natural Ebolavirus Infection. Cell 164, 392-405 (2016).
12. Bornholdt, Z. A. et al. Isolation of potent neutralizing antibodies from a survivor of the 2014 Ebola virus outbreak. Sci. (New York, N.Y.) 351, 1078-1083 (2016).
13. Spadiut, O., Capone, S., Krainer, F., Glieder, A. & Herwig, C. Microbials for the production of monoclonal antibodies and antibody fragments. Trends Biotechnol. 32, 54-60 (2014).
14. Potgieter, T. I. et al. Production of monoclonal antibodies by glycoengineered Pichia pastoris. J. Biotechnol. 139, 318-325 (2009).
15. Vogl, T., Hartner, F. S. & Glieder, A. New opportunities by synthetic biology for biopharmaceutical production in Pichia pastoris. Curr. Opin. Biotechnol. 24, 1094-1101 (2013).
16. Nett, J. H., Gomathinayagam, S. & Hamilton, S. R. Optimization of erythropoietin production with controlled glycosylation-PEGylated erythropoietin produced in glycoengineered Pichia pastoris. J. { . . . } (2012).
17. Hartwig, D. D. et al. High yield expression of leptospirosis vaccine candidates LigA and LipL32 in the methylotrophic yeast Pichia pastoris. Microb. Cell Fact. 9, 98 (2010).
18. Li, H. et al. Optimization of humanized IgGs in glycoengineered Pichia pastoris. Nat. Biotechnol 24, 210-215 (2006).
19. Wildt, S. & Gerngross, T. U. The humanization of N-glycosylation pathways in yeast. Nat. Rev. Microbiol. 3, 119-128 (2005).
20. Hamilton, S. R. et al. Humanization of yeast to produce complex terminally sialylated glycoproteins. Sci. (New York, N.Y.) 313, 1441-1443 (2006).
21. Jacobs, P. P., Geysens, S., Vervecken, W., Contreras, R. & Callewaert, N. Engineering complex-type N-glycosylation in Pichia pastoris using GlycoSwitch technology. Nat. Protoc. 4, 58-70 (2009).
22. Zhang, N. et al. Glycoengineered Pichia produced anti-HER2 is comparable to trastuzumab in preclinical study. MAbs 3, 289-298 (2011).
23. Perez-Pinera, P. et al. Synthetic biology and microbioreactor platformsfor programmable production of biologics at thepoint-of-care. Nat. Commun. 7, 1-10 (2016).
24. Goetze, A. M. et al. High-mannose glycans on the Fc region of therapeutic IgG antibodies increase serum clearance in humans. Glycobiology 21, 949-959 (2011).
25. Maurer, M., Kühleitner, M., Gasser, B. & Mattanovich, D. Versatile modeling and optimization of fed batch processes for the production of secreted heterologous proteins with Pichia pastoris. Microb. Cell Fact. 5, 37 (2006).
26. Cunha, A. E. et al. Methanol induction optimization for scFv antibody fragment production in Pichia pastoris. Biotechnol. Bioeng. 86, 458-467 (2004).
27. Shah, K. A. et al. Automated pipeline for rapid production and screening of HIV-specific monoclonal antibodies using pichia pastoris. Biotechnol. Bioeng. 112, 2624-2629 (2015).
28. Shi, X., Karkut, T., Chamankhah, M. & Alting-Mees, M. Optimal conditions for the expression of a single-chain antibody (scFv) gene in Pichia pastoris. Protein Expr. { . . . } (2003).
29. Szymczak-Workman, A. L., Vignali, K. M. & Vignali, D. A. A. Design and construction of 2A peptide-linked multicistronic vectors. Cold Spring Harb. Protoc. 2012, 199-204 (2012).
30. Qiu, X. et al. Characterization of Zaire ebolavirus glycoprotein-specific monoclonal antibodies. Clin. Immunol. 141, 218-227 (2011).
31. Stettler, K. et al. Specificity, cross-reactivity and function of antibodies elicited by Zika virus infection. Sci. (New York, N.Y.) (2016).
32. http://mappbio.com/zmapp-is-granted-fast-track-status-by-the-fda/.
33. Quick, J. et al. Real-time, portable genome sequencing for Ebola surveillance. Nature 530, 228-232 (2016).
34. Gibson, D. G. et al. Enzymatic assembly of DNA molecules up to several hundred kilobases. Nat. Methods 6, 343-345 (2009).
35. Jostock, T. et al. Combination of the 2A/furin technology with an animal component free cell line development platform process. Appl. Microbiol. Biotechnol. 87, 1517-1524 (2010).
36. Fang, J., Qian, J. J., Yi, S., Harding, T. C. & Tu, G. H. Stable antibody expression at therapeutic levels using the 2A peptide: Abstract: Nature Biotechnology. Nature (2005).
37. Pichia Expression Kit. User Guide. Invit. Life Technol.
38. Johannes F Buyel, H. M. G. R. F. Depth Filters Containing Diatomite Achieve More Efficient Particle Retention than Filters Solely Containing Cellulose Fibers. Front. Plant Sci. 6, (2015).
39. Shukla, A. A. & Hinckley, P. Host cell protein clearance during protein a chromatography: Development of an improved column wash step. Biotechnol. Prog. 24, 1115-1121 (2008).

Having thus described several aspects of at least one embodiment of this invention, it is to be appreciated various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. In addition, any combination of two or more of such features, systems, articles, materials, kits, and/or methods, if such features, systems, articles, materials, kits, and/or methods are not mutually inconsistent, is included within the inventive scope of the present disclosure.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or,” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited. All references, patents and patent applications disclosed herein are incorporated by reference with respect to the subject matter for which each is cited, which in some cases may encompass the entirety of the document.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A method for producing a polypeptide, comprising
(i) providing a genetically modified cell that encodes a first inducible system at a first genetic locus of the cell, wherein the first inducible system comprises a first transcription factor, at least one binding site for the first transcription factor operably linked to a first inducible promoter, and a first recombination site downstream of the first inducible promoter;
(ii) providing to the cell a plasmid that comprises a nucleotide sequence encoding a first polypeptide, optionally a first signal peptide; and a second recombination site;
(iii) expressing a first recombinase compatible with the first and second recombination sites such that recombination occurs between the first recombination site of the cell and the second recombination site of the plasmid resulting in integration of the nucleotide sequence encoding the first polypeptide and optionally the first signal peptide downstream of the first inducible promoter;
(iv) culturing the cell of (iii); and
(v) providing an inducer for the first inducible system thereby inducing expression of the first polypeptide.

2. The method of claim 1, wherein the genetically modified cell encodes a second inducible system at the first genetic locus of the cell.

3. The method of claim 1, wherein the genetically modified cell encodes a second inducible system at a second genetic locus of the cell.

4. The method of claim 2 or 3, wherein the second inducible system comprises a second transcription factor, at least one binding site for the second transcription factor operably linked to a second inducible promoter, and a third recombination site downstream of the second inducible promoter.

5. The method of any one of claims 2-4, wherein the method further comprises
(a) providing to the cell a plasmid that comprises a nucleotide sequence encoding a second polypeptide, optionally a second signal peptide, and a fourth recombination site;
(b) expressing a second recombinase compatible with the third and fourth recombination sites such that recombination occurs between the third recombination site of the cell and the fourth recombination site of the plasmid resulting in integration of the nucleotide sequence encoding the second polypeptide and optionally the second signal peptide downstream of the second inducible promoter;
(c) culturing the cell of (b); and
(d) providing an inducer for the second inducible system thereby inducing expression of the second polypeptide.

6. The method of any one of claims 1-5, wherein the genetically modified cell further encodes a fifth recombination site and the plasmid further comprises a sixth recombination site.

7. The method of claim 6, further comprising expressing a third recombinase compatible with the fifth and sixth recombination sites such that recombination occurs between the fifth and sixth recombination sites resulting in removal of nucleic acid.

8. The method of any one of claims 1-7, wherein the first and second promoters are different.

9. The method of any one of claims 1-8, further comprising collecting the first polypeptide and/or the first and second polypeptides.

10. The method of any one of claims 1-9, further comprising purifying the the first polypeptide and/or the first and second polypeptides.

11. The method of any one of claims 1-10, wherein purifying the first polypeptide and/or second polypeptides comprises obtaining a culture, culture supernatant or composition comprising the first polypeptide and/or second polypeptides, subjecting the culture, culture supernatant or composition comprising the first polypeptide and/or second polypeptides to one or more chromatography steps to purify the first polypeptide and/or the first and second polypeptides.

12. The method of claim 11, wherein the one or more chromatography steps comprise one or more of Sepharose chromatography; reverse phase chromatography, Protein A chromatography, and affinity chromatography.

13. The method of any one of claims 1-12, wherein the cell is a yeast cell.

14. The method of claim 13, wherein the yeast cell is a Komagataella phaffi (Pichia pastoris).

15. The method of claim 13 or 14, wherein the first and/or the second inducible system is on chromosome 2 of the cell.

16. The method of claim 15, wherein the first and/or the second inducible system is at the TRP2 locus of chromosome 2.

17. The method of any one of claims 1-16, wherein the first recombinase, second recombinase, and/or third recombinase is BxbI, R4, TP-901, Cre, Flp, PiggyBac, PhiC31, Gin, Tn3, ParA, HP1, or HK022.

18. The method of any one of claims 1-17, wherein the first recombination site is an attB site, and the second recombination site is an attP site; or the first recombination site is an attP site, and the second recombination site is art attB site.

19. The method of any one of claims 1-18, wherein the DNA binding domain of the first and/or second transcription factor is a zinc finger DNA binding domain.

20. The method of claim 19, wherein the zinc finger DNA binding domain is ZF43-8.

21. The method of any one of claims 1-20, wherein the inducer binding domain of the first and/or second transcription factor is a β-estradiol binding domain.

22. The method of claim 21, wherein the β-estradiol binding domain is from the human estrogen receptor.

23. The method of any one of claims 1-22, wherein the transcription activation domain of the first and/or second transcription factor is VP64.

24. The method of any one of claims 1-23, wherein the inducer of the first and/or second inducible system is β-estradiol.

25. The method of claim 24, wherein the β-estradiol is provided at a concentration of about 0.01 μM-1.0 μM.

26. The method of claim 24 or 25, wherein the β-estradiol is provided for less than 48 hours.

27. The method of claim 26, wherein 0.01 μM β-estradiol is provided for approximately 24 hours.

28. The method of any one of claims 1-27, wherein the plasmid comprises more than one nucleotide sequence encoding more than one polypeptide separated by a nucleotide sequence encoding a 2A peptide.

29. The method of any one of claims 1-28, wherein between 1 pg and 10 g of the first and/or second polypeptide is produced.

30. The method of any one of claims 1-29, wherein the first and/or second polypeptide is a therapeutic molecule.

31. The method of claim 30, wherein the therapeutic molecule is an antibody, hormone, cytokine, chemokine, growth factor, vaccine, or enzyme.

32. The method of claim 31, wherein the cytokine is IFNα2b.

33. The method of claim 31, wherein the growth factor is human growth hormone (hGH).

34. The method of claim 32, wherein at least 19 ug of IFNα2b is produced in approximately 20 hours.

35. The method of claim 33, wherein at least 40 μg man growth hormone is produced in approximately 20 hours.

36. The method of any one of claims 1-35, wherein the first and or second inducible system comprises between 2-9 transcription factor binding sites located upstream of the inducible promoter in the plus orientation or the minus orientation.

37. The method of any one of claims 1-36, wherein expression of the first and/or second transcription factor is regulated by a constitutive promoter.

38. The method of claim 37, wherein the constitutive promoter is a GAP promoter, a TEF1 promoter, a P GCW14 promoter, a variant of the GAP promoter, or a variant of the TEF1 promoter.

39. The method of any one of claims 1-38, wherein the first and/or second inducible promoter is an AOX1 promoter, a GAP promoter, a TEF1 promoter, a P GCW14 promoter, a variant of the GAP promoter, or a variant of the TEF1 promoter.

40. The method of claim 38 or 39, wherein the variant of the TER promoter is a scTEF1 promoter.

41. The method of any one of claims 38-40, wherein
the constitutive promoter is the GAP promoter and the inducible promoter is the AOX1 promoter; or
the constitutive promoter is a variant of the GAP promoter and inducible promoter is the AOX1 promoter; or
the constitutive promoter is the scTEF1 promoter and the inducible promoter is the GAP promoter; or
the constitutive promoter is the scTEF1 promoter and the inducible promoter is a variant of the GAP promoter.

42. The method of any one of claims 1-41, wherein the signal peptide is a yeast signal peptide.

43. The method of claim 42, wherein the yeast signal peptide is a S. cerevisiae signal peptide.

44. The method of claim 43, wherein the yeast signal peptide is the S. cerevisiae mating factor alpha-1 signal peptide.

45. The method of any one of claims 1-44, wherein the first recombinase, second recombinase, and/or third recombinase are encoded on a second plasmid provided to the cell or in the genome of the cell.

46. The method of any one of claims 1-45, wherein the culturing is performed in the presence of at least one antifoam agent.

47. The method of claim 46, wherein the antifoam agent is L81, P2000, or antifoam 204.

48. The method of any one of claims 1-47, further comprising genetically modifying a cell to integrated a first recombination site at the first genetic locus of the cell prior to (i), thereby producing the genetically modified cell.

49. A cell comprising a first nucleic acid encoding
a first transcription factor regulated by a first constitutive promoter,
at least one transcription factor binding site,
a first inducible promoter, and
a nucleotide sequence encoding a first polypeptide, and optionally a first signal peptide, downstream of and operably linked to the first inducible promoter, and
wherein the nucleotide sequence encoding the first polypeptide and optionally the first signal peptide are flanked by a first pair of recombined recombination sites, and wherein the first nucleic acid is located at a first genetic locus, wherein the first genetic locus is in chromosome 2 of the cell.

50. The cell of claim 49, wherein the cell further comprises a second nucleic acid encoding a second transcription factor regulated by a second constitutive promoter,
at least one transcription factor binding site,
a second inducible promoter, and
a nucleotide sequence encoding a second polypeptide, and optionally a second signal peptide, downstream of and operably linked to the second inducible promoter,
wherein the nucleotide sequence encoding the second polypeptide and optionally the second signal peptide are flanked by a second pair of recombined recombination sites, and wherein the second nucleic acid is located at the first locus of the cell.

51. The cell of claim 50, wherein the cell further comprises a second nucleic acid encoding a second transcription factor regulated by a second constitutive promoter,
at least one transcription factor binding site,
a second inducible promoter, and
a nucleotide sequence encoding a second polypeptide, and optionally a second signal peptide, downstream of and operably linked to the second inducible promoter,
wherein the nucleotide sequence encoding the second polypeptide and optionally the second signal peptide are flanked by a second pair of recombined recombination sites, and wherein the second nucleic acid is located at a second locus of the cell.

52. The cell of any one of claims 49-51, wherein the first and second promoters are different.

53. The cell of any one of claims 49-52, wherein the cell is a yeast cell.

54. The cell of claim 53, wherein the yeast cell is a Komagataella phaffi (Pichia pastoris).

55. The cell of any one of claims 49-54, wherein the first and/or the second nucleic acid is at the TRP2 locus of chromosome 2.

56. The cell of any one of claims 49-55, wherein the first and/or second polypeptide is a therapeutic molecule.

57. The cell of claim 56, wherein the therapeutic molecule is an antibody, hormone, cytokine, chemokine, growth factor, vaccine, or enzyme.

58. The cell of claim 57; wherein the cytokine is IFNα2b.

59. The cell of claim 58, wherein the growth factor is human growth hormone (hGH).

60. The cell of any one of claims 49-59, wherein the first and/or second nucleic acid comprises between 2-9 transcription factor binding sites located upstream of the first and/or second inducible promoter in the plus orientation or the minus orientation.

61. The cell of any one of claims 49-60, wherein the first and/or second constitutive promoter is a GAP promoter, a TEF1 promoter, a P GCW14 promoter, a variant of the GAP promoter, or a variant of the TEF1 promoter.

62. The cell of any one of claims 49-61, wherein the first or second inducible promoter is an AOX1 promoter, a GAP promoter, a TEF1 promoter, a P GCW14 promoter, a variant of the GAP promoter, or a variant of the TEF1 promoter.

63. The cell of claim 61 or 62, wherein the variant of the TEF1 promoter is a scTEF1 promoter.

64. The cell of any one of claims 49-63, wherein
the first and/or second constitutive promoter is the GAP promoter and the first or second inducible promoter is the AOX1 promoter; or
the first and/or second constitutive promoter is a variant of the GAP promoter and the first or second inducible promoter is the AOX1 promoter; or
the first and/or second constitutive promoter is the scTEF1 promoter and the first or second inducible promoter is the GAP promoter; or
the first and/or second constitutive promoter is the scTEF1 promoter and the first or second inducible promoter is a variant of the GAP promoter.

65. The cell of any one of claims 49-64, wherein the first and/or second signal peptide is a yeast signal peptide.

66. The cell of claim 65, wherein the yeast signal peptide is a S. cerevisiae signal peptide.

67. The cell of claim 66, wherein the yeast signal peptide is the S. cerevisiae mating factor alpha-1 signal peptide.

68. A method of producing a polypeptide comprising culturing the cell of any one of claims 49-67.

69. The method of claim 68, further comprising providing a first inducer for the first inducible promoter, thereby inducing expression of the first polypeptide.

70. The method of claim 68 or 69, further comprising providing a second inducer for the second inducible promoter, thereby inducing expression of the second polypeptide.

71. The method of any one of claims 68-70, wherein the inducer of the first and/or second inducible promoter is β-estradiol.

72. The method of claim 71, wherein the β-estradiol is provided at a concentration of about 0.01 μM-1.0 μM.

73. The method of claim 71 or 72, wherein the β-estradiol is provided for less than 48 hours.

74. The method of claim 72 or 73, wherein 0.01 μM β-estradiol is provided for approximately 24 hours.

75. The method of any one of claims 68-74, wherein between 1 pg and 10 g of the first and/or second polypeptide is produced.

76. The method of claim 75, wherein at least 19 μg of IFNα2b is produced in approximately 20 hours.

77. The method of claim 75, wherein at least 40 μg human growth hormone is produced in approximately 20 hours.

78. The method of any one of claims 68-77, wherein the culturing is performed in the presence of at least one antifoam agent.

79. The method of claim 78, wherein the antifoam agent is L81, P2000, or antifoam 204.

80. The method of any one of claims 68-79, further comprising collecting the cell culture supernatant.

81. The method of any one of claims 68-80, further comprising purifying the first polypeptide and/or the second polypeptide from the cell culture supernatant.

82. The method of claim 81, wherein purifying the first polypeptide and/or the second polypeptide comprises subjecting the cell culture supernatant comprising the first polypeptide anchor the second polypeptide to one or more chromatography steps to purify the first polypeptide and/or the second polypeptide.

83. The method of claim 82, wherein the one or more chromatography steps comprises one or more of Sepharose chromatography, reverse phase chromatography, Protein A chromatography, and affinity chromatography.

84. A cell culture produced by culturing the cell of any one of claims 49-67.

85. The cell culture of claim 84, wherein the cell culture comprises at between 1 pg and 10 g of the first and/or second polypeptide.

86. A genetically modified cell comprising
a first inducible system comprising a first transcription factor,
at least one transcription factor binding site,
a first inducible promoter, and
a first recombination site downstream of and operably linked to the first inducible promoter, at a first genetic locus;
wherein the first genetic locus is on chromosome 2 of the cell and the cell is a Komagataella phaffi (Pichia pastoris) cell.

87. The cell of claim 86, wherein the cell further comprises a second inducible system comprising a second transcription factor, at least one transcription factor binding site, a second inducible promoter, and a second recombination site downstream of and operably, linked to the second inducible promoter, at the first genetic locus.

88. The cell of claim 86, wherein the cell further comprises a second inducible system comprising a second transcription factor, at least one transcription factor binding site, a second inducible promoter, and a second recombination site downstream of and operably linked to the second inducible promoter, at a second genetic locus.

89. The cell of any one of claims 86-88, wherein the first and/or second genetic locus is the TRP2 locus of chromosome 2.

90. The cell of any one of claims 86-89, wherein the first and/or second inducible systems comprise between 2-9 transcription factor binding sites.

91. The cell of any one of claims 86-90, wherein the first or second inducible promoter is an AOX1 promoter, a GAP promoter, a TEF1 promoter, a P GCW14 promoter, a variant of the GAP promoter, or a variant of the TEF1 promoter.

92. The cell of claim 91, wherein the variant of the TEF1 promoter is a scTEF1 promoter.

93. A kit comprising
(i) a genetically modified cell of any one of claims 86-92,
(ii) a first recombinase, and
(iii) a first plasmid encoding a first polypeptide, optionally a first signal peptide, and a second recombination site.

94. The kit of claim 93, further comprising
(iv) a second recombinase, and
(v) a second plasmid encoding a second polypeptide, optionally a second signal peptide and a third recombination site.

95. A method for producing a therapeutic antibody comprising
isolating B cells from infected individuals,
determining the sequence of antibody variable regions from the B cells isolated from from the infected individuals,
synthesizing one or more antibodies using the antibody variable region sequences,
engineering strains of Komagataella phaffi to express the one or more antibodies, and
culturing the engineered strains of Komagataella phaffi to produce the one or more antibodies.

96. The method of claim 95, further comprising purifying the one or more antibodies.

97. The method of claim 95 or claim 96, further comprising screening for highly productive engineered strains of Komagataella phaffi that produce the one or more antibodies.

98. A method for treating an infection comprising administering antibodies made by the method of any one of claims 95-97 to a subject in need of such treatment.


SEQUENCE LISTING (30 sequences)

SEQ ID NO: 1 - DNA, 4262 bp, Artificial Sequence
  Synthetic polynucleotide
caccatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaaatttttttttttgatttttttctctttcgatgacctcccattgatatttaagttaataaacggtcttcaatttctcaagtttcagtttcatttttcttgttctattacaactttttttacttcttgctcattagaaagaaagcatagcaatctaatctaagggcggtgttgacaattaatcatcggcatagtatatcggcatagtataatacgacaaggtgaggaactaaaccatggtaatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaacacatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagctggagaccaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcatgagatcagatctgaagttcctatactttctagagaataggaacttcaagcttgtggaacattgagaccaaacaagactcgcttcgatgctttcagatccattttcccagcaggtaccgtctccggtgctccgaaggtaagagcaatgcaactcataggagaattggaaggagaaaagagaggtgtttatgcgggggccgtaggacactggtcgtacgatggaaaatcgatggacacatgtattgccttaagaacaatggtcgtcaaggacggtgtcgcttaccttcaagccggaggtggaattgtctacgattctgacccctatgacgagtacatcgaaaccatgaacaaaatgagatccaacaataacaccatcttggaggctgagaaaatctggaccgataggttggccagagacgagaatcaaagtgaatccgaagaaaacgatcaatgaacggaggacgtaagtaggaatttatgtaatcatgccaatacatctttagatttcttcctcttctttttcatgagattattggaaaccaccagaatcgaatataaaaggcgaacacctttcccaattttggtttctcctgacccaaagactttaaatttaatttatttgtccctatttcaatcaattgaacaactatttcggctggacggcgacgtaaacggccacaagtttatggccgtgatgacctgtgtcttcgtggtttgtctggtcaaccaccgcggtctcagtggtgtacggtacaaacccaaagcagcacgacacggcaactacaagacccgcgccgagggcatgttccccaaagcgataccacttgaagcagtggtactgcttgtgggtacactctgcgggtgtgaagttcgagggcgacaccctggtgaaccgcatcgagctgaagggcatcttccaactcgcttaattgcgagtttttatttcgtttatttcaattaaggtaactaaaaaactccttttacacatgaagcagcacgacttcttcaagtccgccatgcccgaaaaacgcctcttcagagtacagaagattaagtgagaccttcgtttgtgcggatcccccacacaccatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaaatttttttttttgatttttttctctttcgatgacctcccattgatatttaagttaataaacggtcttcaatttctcaagtttcagtttcatttttcttgttctattacaactttttttacttcttgctcattagaaagaaagcatagcaatctaatctaagggcggtgttgacaattaatcatcggcatagtatatcggcatagtataatacgacaaggtgaggaactaaaccatggtaatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgt

SEQ ID NO: 2 - DNA, 4474 bp, Artificial Sequence
  Synthetic polynucleotide
gactcttcgcgatgtacgggccagatatacgcgttgacattgattattgactagtccacacaccatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaaatttttttttttgatttttttctctttcgatgacctcccattgatatttaagttaataaacggtcttcaatttctcaagtttcagtttcatttttcttgttctattacaactttttttacttcttgctcattagaaagaaagcatagcaatctaatctaaggctagcgtttaaaccaccatgagggcccttgtagttatcaggttgagtagggttacggatgcaaccaccagcccggagcgccaactggaatcatgtcagcagctttgtgcgcagcgcggctgggacgtggtgggagtggcggaggacttggacgttagcggggccgttgacccatttgaccgaaagcggagacctaacctggctcgatggctcgcctttgaggagcagcccttcgatgtgatcgtcgcatacagggtcgacagactgaccagatccattcgccatctgcagcagctcgttcactgggcggaggaccacaaaaagctcgtggtgagtgcaacagaagcccactttgacaccacaacacccttcgcagccgtcgtgatcgctctgatgggtaccgttgcccagatggaattggaggcaatcaaggagcggaacagatccgccgctcatttcaatatccgcgcgggcaagtacaggggtagtctcccaccctgggggtatttgcctacccgggtggacggcgaatggaggcttgttcccgatcccgtgcagcgagagcgaatactggaagtttatcatcgagtcgtggataaccatgaaccactccacctggtggcccacgaccttaaccgacgcggcgtgctgagccctaaggactattttgctcaacttcagggaagagagccacagggtagggaatggtcagccacagctctcaagcggtctatgatttccgaagcaatgctcgggtacgcaacactcaatggcaagacagttcgagacgacgacggggcccccctggttcgggccgaacccatacttacccgcgaacaactggaggcacttcgcgcggaacttgtgaaaacaagccgagccaaacccgcagtgagcaccccatcactgctgctgagggtgctcttctgtgccgtgtgcggcgaaccagcatacaagttcgctggcgggggtcgaaaacacccccgctaccggtgtcgctcaatgggttttccaaagcactgtggcaacggaacagttgcaatggccgaatgggacgctttttgtgaagaacaagtgctggatcttctgggcgacgctgagaggctggaaaaagtatgggtggccgggagcgacagcgccgttgagctcgccgaggtgaacgccgaattggtggacctgacgagtctcatcggatctccagcataccgagctggatccccccagcgagaggctctggacgctcggatagccgccctggcagcaaggcaggaggagcttgaggggttggaagcacggccttcaggatgggaatggcgggaaacaggacagagatttggagactggtggagggaacaggataccgctgctaagaacacttggctcaggtccatgaatgttcgactcaccttcgacgtgaggggtgggttgacccgcaccattgatttcggggatctgcaggagtatgaacagcatctccggcttggctccgtggtagaaagacttcatacaggcatgtcatgaagatctattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagctggagaccaacatgtgagcaaaaggccagcatctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttctt

SEQ ID NO: 3 - DNA, 6635 bp, Artificial Sequence
  Synthetic polynucleotide
ggtctgacgctcagtggaacgaaaactcacgttaagggattttggtcatgagatcagatctgagctcgtttggccgtggccgtgctcgtcctcgtcggccggcttgtcgacgacggcggtctccgtcgtcaggatcatccgggccacaagcttgctgacagaagcctcaagaaaaaaaaaattcttcttcgactatgctggaggcagagatgatcgagccggtagttaactatatatagctaaattggttccatcacctcgagtcaattcttgatttagtatacacataaccaaatttggatcaagtttgaagtaaaactttaacttcagctccttacatttgcactaagatctctgctactctggtcccaagtgaaccaccttttggaccctattgaccggaccttaacttgccaaacctaaacgcttaatgcctcagacgttttaatgcctctcaacacctccaaggttgctttcttgagcatgcctactaggaactttaacgaactgtggggttgcagacagtttcaggcgtgtcccgaccaatatggcctactagactctctgaaaaatcacagttttccagtagttccgatcaaattaccatcgaaatggtcccataaacggacatttgacatccgttcctgaattatagtcttccaccgtggatcatggtgttcctttttttcccaaagaatatcagcatcccttaactacgttaggtcagtgatgacaatggaccaaattgttgcaaggtttttctttttctttcatcggcacatttcagcctcacatgcgactattatcgatcaatgaaatccatcaagattgaaatcttaaaattgcccctttcacttgacaggatccttttttgtagaaatgtcttggtgtcctcgtccaatcaggtagccatctctgaaatatctggctccgttgcaactccgaacgacctgctggcaacgtaaaattctccggggtaaaactttaatgtggagtaatggaaccagaaacgtctcttcccttctctctccttccaccgcccgttaccgtccctaggaaattttactctgctggagagcttcttctacggcccccttgcagcaatgctcttcccagcattacgttgcgggtaaaacggaggtcgtgtacccgacctagcagcccagggatggaaaagtcccggccgtcgctggcaataatagcgggcggacgcatgtcatgagattattggaaaccaccagaatcgaatataaaaggcgaacacctttcccaattttggtttcccctgacccaaagactttaaatttaatttatttgtccctatttcaatcaattgaacaactatcaaaacacactagtaaaaatgcgcggagctcctaagaaaaagcgcaaagtccggccggcatctagacccggggagcgccccttccagtgtcgcatttgcatgcggaacttttcgcgccaggacaggcttgacaggcatacccgtactcataccggtgaaaaaccgtttcagtgtcggatctgtatgcgaaatttctcccagaaggagcacttggcggggcatctacgtacgcacaccggcgagaagccattccaatgccgaatatgcatgcgcaacttcagtcgccgcgacaacctgaaccggcacctaaaaacccacctgaggaacatatgcggcggaggcacacctgcagctgcgtcgactctagaggatccatctgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccagtgaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggagatgctggacgcccaccgcctacatgcgcccactagccgtggaggggcatccgtggaggagacggaccaaagccacttggccactgcgggctctacttcatcgcctagggccgacgcgctggacgatttcgatctcgacatgctgggttctgatgccctcgatgactttgacctggatatgttgggaagcgacgcattggatgactttgatctggacatgctcggctccgatgctctggacgatttcgatctcgatatgttaattaactacccgtacgacgttccggactacgcttcttgaggtaccatcggtagaccggtcttgctagattctaatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagagctagcatatcatatagaagtcatcgcggcagatcaattcatcaatcggagtgaggatattgcgagtttaccaccatcaatcggagtgaggatctccaattggtgacggtccagtcatcaatcggagtgaggattcagctgcttctcgaggccgcacatcaatcggagtgaggatgtacagggtgggctgttccaccatcaatcggagtgaggatttgcgtcaatggggcggagttcatcaatcggagtgaggatatcgaagtcatcgagagcactcatcaatcggagtgaggatatactccacccattgacgtcacatcaatcggagtgaggattggaaccagaagggtctcttcatcaatcggagtgaggataccctatgggcgcgcctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacgcgaattcatgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaagagattcccaaccattcccttatctagactttttgacaacgctatgctccgcgcccatcgtctgcaccagctggcctttgacacctaccaggagtttgaagaagcctatatcccaaaggaacagaagtattcattcctgcagaacccccagacctccctctgtttctcagagtctattccgacaccctccaacagggaggaaacacaacagaaatccaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaaggcatccaaacgctgatggggaggctggaagatggcagcccccggactgggcagatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggcagctgtggcttctagcggtcttgctagattctaatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagaccttcgtttgtgcggatcccccacacaccatagcttcaaaatgtttctactccttttttactcttccagattttctcggactccgcgcatcgccgtaccacttcaaaacacccaagcacagcatactaaattttccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaatttttttttttagtttttttctctttcagtgacctccattgatatttaagttaataaacggtcttcaatttctcaagtttcagtttcatttttcttgttctattacaactttttttacttcttgttcattagaaagaaagcatagcaatctaatctaaggggcggtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtccgacggcggcccacgggtcccaggcctcggagatccgtcccccttttcctttgtcgatatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagctggagaccaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacgg

SEQ ID NO: 4 - DNA, 639 bp, Artificial Sequence
  Synthetic polynucleotide
tcagcatctggttacgtaactctggcaaccagtaacacgcttaaggtttggaacaacactaaactaccttgcggtactaccattgacactacacatccttaattccaatcctgtctggcctccttcaccttttaaccatcttgcccattccaactcgtgtcagattgcgtatcaagtgaaaaaaaaaaattttaaaatctttaacccaatcaggtaataactgtcgcctcttttatctgccgcactgcatgaggtgtccccttagtgggaaagagtactgagccaaccctggaggacagcaagggaaaaatacctacaacttgcttcataatggtcgtaaaaacaatccttgtcggatataagtgttgtagactgtcccttatcctctgcgatgttcttcctctcaaagtttgcgatttctctctatcagaattgccatcaagagactcaggactaatttcgcagtcccacacgcactcgtacatgattggctgaaatttccctaaagaatttctttttcacgaaaatttttttttacacaagattttcagcagatataaaatggagagcaggacctccgctgtgactcttcttttttttcttttattctcactacatacattttagttattcgccaac

SEQ ID NO: 5 - DNA, 423 bp, Artificial Sequence
  Synthetic polynucleotide
ataactgtcgcctcttttatctgccgcactgcatgaggtgtccccttagtgggaaagagtactgagccaaccctggaggacagcaagggaaaaatacctacaacttgcttcataatggtcgtaaaaacaatccttgtcggatataagtgttgtagactgtcccttatcctctgcgatgttcttcctctcaaagtttgcgatttctctctatcagaattgccatcaagagactcaggactaatttcgcagtcccacacgcactcgtacatgattggctgaaatttccctaaagaatttctttttcacgaaaatttttttttacacaagattttcagcagatataaaatggagagcaggacctccgctgtgactcttcttttttttcttttattctcactacatacattttagttattcgccaac

SEQ ID NO: 6 - DNA, 407 bp, Artificial Sequence
  Synthetic polynucleotide
ccacacaccatagcttcaaaatgtttctactccttttttactcttccagattttctcggactccgcgcatcgccgtaccacttcaaaacacccaagcacagcatactaaattttccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaatttttttttttagtttttttctctttcagtgacctccattgatatttaagttaataaacggtcttcaatttctcaagtttcagtttcatttttcttgttctattacaactttttttacttcttgttcattagaaagaaagcatagcaatctaatctaagg

SEQ ID NO: 7 - DNA, 822 bp, Artificial Sequence
  Synthetic polynucleotide
caggtgaacccacctaactatttttaactgggatccagtgagctcgctgggtgaaagccaaccatcttttgtttcggggaaccgtgctcgccccgtaaagttaatttttttttcccgcgcagctttaatctttcggcagagaaggcgttttcatcgtagcgtgggaacagaataatcagttcatgtgctatacaggcacatggcagcagtcactattttgctttttaaccttaaagtcgttcatcaatcattaactgaccaatcagattttttgcatttgccacttatctaaaaatacttttgtatctcgcagatacgttcagtggtttccaggacaacacccaaaaaaaggtatcaatgccactaggcagtcggttttatttttggtcacccacgcaaagaagcacccacctcttttaggttttaagttgtgggaacagtaacaccgcctagagcttcaggaaaaaccagtacctgtgaccgcaattcaccatgatgcagaatgttaatttaaacgagtgccaaatcaagatttcaacagacaaatcaatcgatccatagttacccattccagccttttcgtcgtcgagcctgcttcattcctgcctcaggtgcataactttgcatgaaaagtccagattagggcagattttgagtttaaaataggaaatataaacaaatataccgcgaaaaaggtttgtttatagcttttcgcctggtgccgtacggtataaatacatactctcctcccccccctggttctctttttcttttgttacttacattttaccgttccgtcactcgcttcactcaacaacaaaa

SEQ ID NO: 8 - DNA, 1091 bp, Artificial Sequence
  Synthetic polynucleotide
tcaattcttgatttagtatacacataaccaaatttggatcaagtttgaagtaaaactttaacttcagctccttacatttgcactaagatctctgctactctggtcccaagtgaaccaccttttggaccctattgaccggaccttaacttgccaaacctaaacgcttaatgcctcagacgttttaatgcctctcaacacctccaaggttgctttcttgagcatgcctactaggaactttaacgaactgtggggttgcagacagtttcaggcgtgtcccgaccaatatggcctactagactctctgaaaaatcacagttttccagtagttccgatcaaattaccatcgaaatggtcccataaacggacatttgacatccgttcctgaattatagtcttccaccgtggatcatggtgttcctttttttcccaaagaatatcagcatcccttaactacgttaggtcagtgatgacaatggaccaaattgttgcaaggtttttctttttctttcatcggcacatttcagcctcacatgcgactattatcgatcaatgaaatccatcaagattgaaatcttaaaattgcccctttcacttgacaggatccttttttgtagaaatgtcttggtgtcctcgtccaatcaggtagccatctctgaaatatctggctccgttgcaactccgaacgacctgctggcaacgtaaaattctccggggtaaaactttaatgtggagtaatggaaccagaaacgtctcttcccttctctctccttccaccgcccgttaccgtccctaggaaattttactctgctggagagcttcttctacggcccccttgcagcaatgctcttcccagcattacgttgcgggtaaaacggaggtcgtgtacccgacctagcagcccagggatggaaaagtcccggccgtcgctggcaataatagcgggcggacgcatgtcatgagattattggaaaccaccagaatcgaatataaaaggcgaacacctttcccaattttggtttcccctgacccaaagactttaaatttaatttatttgtccctatttcaatcaattgaacaactatcaaaacaca

SEQ ID NO: 9 - DNA, 1131 bp, Artificial Sequence
  Synthetic polynucleotide
tcaattcttgatttagtatacacataaccaaatttggatcaagtttgaagtaaaactttaacttcagctccttacatttgcactaagatctctgctactctggtcccaagtgaaccaccttttggaccctattgaccggaccttaacttgccaaacctaaacgcttaatgcctcagacgttttaatgcctctcaacacctccaaggttgctttcttgagcatgcctactaggaactttaacgaactgtggggttgcagacagtttcaggcgtgtcccgaccaatatggcctactagactctctgaaaaatcacagttttccagtagttccgatcaaattaccatcgaaatggtcccataaacggacatttgacatccgttcctgaattatagtcttccaccgtggatcatggtgttcctttttttcccaaagaatatcagcatcccttaactacgttaggtcagtgatgacaatggaccaaattgttgcaaggtttttctttttctttcatcggcacatttcagcctcacatgcgactattatcgatcaatgaaatccatcaagattgaaatcttaaaattgcccctttcacttgacaggatccttttttgtagaaatgtcttggtgtcctcgtccaatcaggtagccatctctgaaatatctggctccgttgcaactccgaacgacctgctggcaacgtaaaattctccggggtaaaactttaatgtggagtaatggaaccagaaacgtctcttcccttctctctccttccaccgcccgttaccgtccctaggatccctatcagtgatagagatctccctatcagtgatagagaaattttactctgctggagagcttcttctacggcccccttgcagcaatgctcttcccagcattacgttgcgggtaaaacggaggtcgtgtacccgacctagcagcccagggatggaaaagtcccggccgtcgctggcaataatagcgggcggacgcatgtcatgagattattggaaaccaccagaatcgaatataaaaggcgaacacctttcccaattttggtttctcctgacccaaagactttaaatttaatttatttgtccctatttcaatcaattgaacaactatcaaaacaca

SEQ ID NO: 10 - DNA, 1131 bp, Artificial Sequence
  Synthetic polynucleotide
tcaattcttgatttagtatacacataaccaaatttggatcaagtttgaagtaaaactttaacttcagctccttacatttgcactaagatctctgctactctggtcccaagtgaaccaccttttggaccctattgaccggaccttaacttgccaaacctaaacgcttaatgcctcagacgttttaatgcctctcaacacctccaaggttgctttcttgagcatgcctactaggaactttaacgaactgtggggttgcagacagtttcaggcgtgtcccgaccaatatggcctactagactctctgaaaaatcacagttttccagtagttccgatcaaattaccatcgaaatggtcccataaacggacatttgacatccgttcctgaattatagtcttccaccgtggatcatggtgttcctttttttcccaaagaatatcagcatcccttaactacgttaggtcagtgatgacaatggaccaaattgttgcaaggtttttctttttctttcatcggcacatttcagcctcacatgcgactattatcgatcaatgaaatccatcaagattgaaatcttaaaattgcccctttcacttgacaggatccttttttgtagaaatgtcttggtgtcctcgtccaatcaggtagccatctctgaaatatctggctccgttgcaactccgaacgacctgctggcaacgtaaaattctccggggtaaaactttaatgtggagtaatggaaccagaaacgtctcttcccttctctctccttccaccgcccgttaccgtccctaggaaattttactctgctggagagcttcttctacggcccccttgcagcaatgctctccctatcagtgatagagatctccctatcagtgatagagattcccagcattacgttgcgggtaaaacggaggtcgtgtacccgacctagcagcccagggatggaaaagtcccggccgtcgctggcaataatagcgggcggacgcatgtcatgagattattggaaaccaccagaatcgaatataaaaggcgaacacctttcccaattttggtttctcctgacccaaagactttaaatttaatttatttgtccctatttcaatcaattgaacaactatcaaaacaca

SEQ ID NO: 11 - DNA, 1131 bp, Artificial Sequence
  Synthetic polynucleotide
tcaattcttgatttagtatacacataaccaaatttggatcaagtttgaagtaaaactttaacttcagctccttacatttgcactaagatctctgctactctggtcccaagtgaaccaccttttggaccctattgaccggaccttaacttgccaaacctaaacgcttaatgcctcagacgttttaatgcctctcaacacctccaaggttgctttcttgagcatgcctactaggaactttaacgaactgtggggttgcagacagtttcaggcgtgtcccgaccaatatggcctactagactctctgaaaaatcacagttttccagtagttccgatcaaattaccatcgaaatggtcccataaacggacatttgacatccgttcctgaattatagtcttccaccgtggatcatggtgttcctttttttcccaaagaatatcagcatcccttaactacgttaggtcagtgatgacaatggaccaaattgttgcaaggtttttctttttctttcatcggcacatttcagcctcacatgcgactattatcgatcaatgaaatccatcaagattgaaatcttaaaattgcccctttcacttgacaggatccttttttgtagaaatgtcttggtgtcctcgtccaatcaggtagccatctctgaaatatctggctccgttgcaactccgaacgacctgctggcaacgtaaaattctccggggtaaaactttaatgtggagtaatggaaccagaaacgtctcttcccttctctctccttccaccgcccgttaccgtccctaggaaattttactctgctggagagcttcttctacggcccccttgcagcaatgctcttcccagcattacgttgtccctatcagtgatagagatctccctatcagtgatagagacgggtaaaacggaggtcgtgtacccgacctagcagcccagggatggaaaagtcccggccgtcgctggcaataatagcgggcggacgcatgtcatgagattattggaaaccaccagaatcgaatataaaaggcgaacacctttcccaattttggtttctcctgacccaaagactttaaatttaatttatttgtccctatttcaatcaattgaacaactatcaaaacaca

SEQ ID NO: 12 - DNA, 1131 bp, Artificial Sequence
  Synthetic polynucleotide
tcaattcttgatttagtatacacataaccaaatttggatcaagtttgaagtaaaactttaacttcagctccttacatttgcactaagatctctgctactctggtcccaagtgaaccaccttttggaccctattgaccggaccttaacttgccaaacctaaacgcttaatgcctcagacgttttaatgcctctcaacacctccaaggttgctttcttgagcatgcctactaggaactttaacgaactgtggggttgcagacagtttcaggcgtgtcccgaccaatatggcctactagactctctgaaaaatcacagttttccagtagttccgatcaaattaccatcgaaatggtcccataaacggacatttgacatccgttcctgaattatagtcttccaccgtggatcatggtgttcctttttttcccaaagaatatcagcatcccttaactacgttaggtcagtgatgacaatggaccaaattgttgcaaggtttttctttttctttcatcggcacatttcagcctcacatgcgactattatcgatcaatgaaatccatcaagattgaaatcttaaaattgcccctttcacttgacaggatccttttttgtagaaatgtcttggtgtcctcgtccaatcaggtagccatctctgaaatatctggctccgttgcaactccgaacgacctgctggcaacgtaaaattctccggggtaaaactttaatgtggagtaatggaaccagaaacgtctcttcccttctctctccttccaccgcccgttaccgtccctaggaaattttactctgctggagagcttcttctacggcccccttgcagcaatgctcttcccagcattacgttgcgggtaaaacggaggtcgtgtacccgacctagcagcccagggatggaaaagtcccggccgtcgctggcaataatagcgggcgtccctatcagtgatagagatctccctatcagtgatagagagacgcatgtcatgagattattggaaaccaccagaatcgaatataaaaggcgaacacctttcccaattttggtttctcctgacccaaagactttaaatttaatttatttgtccctatttcaatcaattgaacaactatcaaaacaca

SEQ ID NO: 13 - DNA, 1131 bp, Artificial Sequence
  Synthetic polynucleotide
tcaattcttgatttagtatacacataaccaaatttggatcaagtttgaagtaaaactttaacttcagctccttacatttgcactaagatctctgctactctggtcccaagtgaaccaccttttggaccctattgaccggaccttaacttgccaaacctaaacgcttaatgcctcagacgttttaatgcctctcaacacctccaaggttgctttcttgagcatgcctactaggaactttaacgaactgtggggttgcagacagtttcaggcgtgtcccgaccaatatggcctactagactctctgaaaaatcacagttttccagtagttccgatcaaattaccatcgaaatggtcccataaacggacatttgacatccgttcctgaattatagtcttccaccgtggatcatggtgttcctttttttcccaaagaatatcagcatcccttaactacgttaggtcagtgatgacaatggaccaaattgttgcaaggtttttctttttctttcatcggcacatttcagcctcacatgcgactattatcgatcaatgaaatccatcaagattgaaatcttaaaattgcccctttcacttgacaggatccttttttgtagaaatgtcttggtgtcctcgtccaatcaggtagccatctctgaaatatctggctccgttgcaactccgaacgacctgctggcaacgtaaaattctccggggtaaaactttaatgtggagtaatggaaccagaaacgtctcttcccttctctctccttccaccgcccgttaccgtccctaggaaattttactctgctggagagcttcttctacggcccccttgcagcaatgctcttcccagcattacgttgcgggtaaaacggaggtcgtgtacccgacctagcagcccagggatggaaaagtcccggccgtcgctggcaataatagcgggcggacgcatgtcatgagattattggaaaccaccagaatcgaatataaaaggcgtccctatcagtgatagagatctccctatcagtgatagagaaacacctttcccaattttggtttctcctgacccaaagactttaaatttaatttatttgtccctatttcaatcaattgaacaactatcaaaacaca

SEQ ID NO: 14 - DNA, 1131 bp, Artificial Sequence
  Synthetic polynucleotide
tcaattcttgatttagtatacacataaccaaatttggatcaagtttgaagtaaaactttaacttcagctccttacatttgcactaagatctctgctactctggtcccaagtgaaccaccttttggaccctattgaccggaccttaacttgccaaacctaaacgcttaatgcctcagacgttttaatgcctctcaacacctccaaggttgctttcttgagcatgcctactaggaactttaacgaactgtggggttgcagacagtttcaggcgtgtcccgaccaatatggcctactagactctctgaaaaatcacagttttccagtagttccgatcaaattaccatcgaaatggtcccataaacggacatttgacatccgttcctgaattatagtcttccaccgtggatcatggtgttcctttttttcccaaagaatatcagcatcccttaactacgttaggtcagtgatgacaatggaccaaattgttgcaaggtttttctttttctttcatcggcacatttcagcctcacatgcgactattatcgatcaatgaaatccatcaagattgaaatcttaaaattgcccctttcacttgacaggatccttttttgtagaaatgtcttggtgtcctcgtccaatcaggtagccatctctgaaatatctggctccgttgcaactccgaacgacctgctggcaacgtaaaattctccggggtaaaactttaatgtggagtaatggaaccagaaacgtctcttcccttctctctccttccaccgcccgttaccgtccctaggaaattttactctgctggagagcttcttctacggcccccttgcagcaatgctcttcccagcattacgttgcgggtaaaacggaggtcgtgtacccgacctagcagcccagggatggaaaagtcccggccgtcgctggcaataatagcgggcggacgcatgtcatgagattattggaaaccaccagaatcgaatataaaaggcgaacacctttcccaattttggtttctcctgacccaaagactttaaatttccctatcagtgatagagatctccctatcagtgatagagataatttatttgtccctatttcaatcaattgaacaactatcaaaacaca

SEQ ID NO: 15 - DNA, 1155 bp, Artificial Sequence
  Synthetic polynucleotide
tcaattcttgatttagtatacacataaccaaatttggatcaagtttgaagtaaaactttaacttcagctccttacatttgcactaagatctctgctactctggtcccaagtgaaccaccttttggaccctattgaccggaccttaacttgccaaacctaaacgcttaatgcctcagacgttttaatgcctctcaacacctccaaggttgctttcttgagcatgcctactaggaactttaacgaactgtggggttgcagacagtttcaggcgtgtcccgaccaatatggcctactagactctctgaaaaatcacagttttccagtagttccgatcaaattaccatcgaaatggtcccataaacggacatttgacatccgttcctgaattatagtcttccaccgtggatcatggtgttcctttttttcccaaagaatatcagcatcccttaactacgttaggtcagtgatgacaatggaccaaattgttgcaaggtttttctttttctttcatcggcacatttcagcctcacatgcgactattatcgatcaatgaaatccatcaagattgaaatcttaaaattgcccctttcacttgacaggatccttttttgtagaaatgtcttggtgtcctcgtccaatcaggtagccatctctgaaatatctggctccgttgcaactccgaacgacctgctggcaacgtaaaattctccggggtaaaactttaatgtggagtaatggaaccagaaacgtctcttcccttctctctccttccaccgcccgttaccgtccctaggaaattttactctgctggagagcttcttctacggcccccttgcagcaatgctcttcccagcattacgttgcgggtaaaacggaggtcgtgtacccgacctagcagcccagggatggaaaagtcccggccgtcgctggcaataatagcgggcggacgcatgtcatgagattattggaaaccaccagaatcgaatataaaaggcgaacacctttcccaattttggtttcccctgacccaaagactttaaatttaatttatttgtccctatttcaatcaattgaacaactatcaaaacacagaatttccctatcagtgatagagatctccctatcagtgatagagagaattcatggtgagcaagg

SEQ ID NO: 16 - DNA, 20 bp, Artificial Sequence
  Synthetic polynucleotide
gggcagatcttcaagcagac

SEQ ID NO: 17 - DNA, 20 bp, Artificial Sequence
  Synthetic polynucleotide
ctcgaccttgtccatgtcct

SEQ ID NO: 18 - DNA, 20 bp, Artificial Sequence
  Synthetic polynucleotide
ttcccacaagaggaatttgg

SEQ ID NO: 19 - DNA, 20 bp, Artificial Sequence
  Synthetic polynucleotide
aggctgctgaggaatctttg

SEQ ID NO: 20 - DNA, 20 bp, Artificial Sequence
  Synthetic polynucleotide
tgggttacactgaagatgcc

SEQ ID NO: 21 - DNA, 22 bp, Artificial Sequence
  Synthetic polynucleotide
cgttgtcgtaccaagagatcag

SEQ ID NO: 22 - DNA, 21 bp, Artificial Sequence
  Synthetic polynucleotide
tggtatcgttttggactctgg

SEQ ID NO: 23 - DNA, 20 bp, Artificial Sequence
  Synthetic polynucleotide
agcgtgtggtaaggagaaac

SEQ ID NO: 24 - DNA, 20 bp, Artificial Sequence
  Synthetic polynucleotide
tgtcttcgtggtttgtctgg

SEQ ID NO: 25 - DNA, 19 bp, Artificial Sequence
  Synthetic polynucleotide
tcttgtagttgccgtgtcg

SEQ ID NO: 26 - protein, 18 aa, Artificial Sequence
  Synthetic polypeptide
EGRGSLLTCGDVEENPGP

SEQ ID NO: 27 - protein, 19 aa, Artificial Sequence
  Synthetic polypeptide
ATNFSLLKQAGDVEENPGP

SEQ ID NO: 28 - protein, 20 aa, Artificial Sequence
  Synthetic polypeptide
QCTNYALLKLAGDVESNPGP

SEQ ID NO: 29 - protein, 22 aa, Artificial Sequence
  Synthetic polypeptide
VKQTLNFDLLKLAGDVESNPGP

SEQ ID NO: 30 - protein, 7 aa, Artificial Sequence
  TEV protease cleavage site
  misc_feature (7)..(7): Xaa can be any naturally occurring amino acid except Proline
ENLYFQX